SEQUENCE LISTING (178 sequences)

SEQ ID NO: 1 - protein, 9 aa, synthetic construct
TGAVTSGNY

SEQ ID NO: 2 - skipped

SEQ ID NO: 3 - protein, 9 aa, synthetic construct
VLWYSNRWV

SEQ ID NO: 4 - protein, 9 aa, synthetic construct
TGAVTSANY

SEQ ID NO: 5 - protein, 9 aa, synthetic construct
ALWYSNRWV

SEQ ID NO: 6 - protein, 9 aa, synthetic construct
VAWYSNRWV

SEQ ID NO: 7 - protein, 9 aa, synthetic construct
VLAYSNRWV

SEQ ID NO: 8 - protein, 9 aa, synthetic construct
VLWASNRWV

SEQ ID NO: 9 - protein, 9 aa, synthetic construct
VLWYANRWV

SEQ ID NO: 10 - protein, 9 aa, synthetic construct
VLWYSARWV

SEQ ID NO: 11 - protein, 9 aa, synthetic construct
VLWYSNAWV

SEQ ID NO: 12 - protein, 9 aa, synthetic construct
VLWYSNRAV

SEQ ID NO: 13 - protein, 9 aa, synthetic construct
VLWYSNRWA

SEQ ID NO: 14 - protein, 10 aa, synthetic construct
VLWYSNRWVA

SEQ ID NO: 15 - protein, 8 aa, synthetic construct
GFTFNKYA

SEQ ID NO: 16 - protein, 10 aa, synthetic construct
IRSKYNNYAT

SEQ ID NO: 17 - protein, 16 aa, synthetic construct
VRHGNFGNSYISYWAY

SEQ ID NO: 18 - protein, 8 aa, synthetic construct
GFTFQKYA

SEQ ID NO: 19 - protein, 16 aa, synthetic construct
ARHGNFGNSYISYWAY

SEQ ID NO: 20 - protein, 16 aa, synthetic construct
VAHGNFGNSYISYWAY

SEQ ID NO: 21 - protein, 16 aa, synthetic construct
VRAGNFGNSYISYWAY

SEQ ID NO: 22 - protein, 16 aa, synthetic construct
VRHANFGNSYISYWAY

SEQ ID NO: 23 - protein, 16 aa, synthetic construct
VRHGAFGNSYISYWAY

SEQ ID NO: 24 - protein, 16 aa, synthetic construct
VRHGNAGNSYISYWAY

SEQ ID NO: 25 - protein, 16 aa, synthetic construct
VRHGNFANSYISYWAY

SEQ ID NO: 26 - protein, 16 aa, synthetic construct
VRHGNFGASYISYWAY

SEQ ID NO: 27 - protein, 16 aa, synthetic construct
VRHGNFGNAYISYWAY

SEQ ID NO: 28 - protein, 16 aa, synthetic construct
VRHGNFGNSAISYWAY

SEQ ID NO: 29 - protein, 16 aa, synthetic construct
VRHGNFGNSYASYWAY

SEQ ID NO: 30 - protein, 16 aa, synthetic construct
VRHGNFGNSYIAYWAY

SEQ ID NO: 31 - protein, 16 aa, synthetic construct
VRHGNFGNSYISAWAY

SEQ ID NO: 32 - protein, 16 aa, synthetic construct
VRHGNFGNSYISYAAY

SEQ ID NO: 33 - protein, 16 aa, synthetic construct
VRHGNFGNSYISYWAA

SEQ ID NO: 34 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 35 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 36 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 37 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCARHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 38 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVAHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 39 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 41 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGAFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 42 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 43 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFANSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGASYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 45 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 46 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSAISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 47 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYASYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 48 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYIAYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 49 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISAWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 50 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYAAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 51 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAAWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCALWYSNRWVFGGGTKLTVL

SEQ ID NO: 53 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVL

SEQ ID NO: 54 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLAYSNRWVFGGGTKLTVL

SEQ ID NO: 55 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWASNRWVFGGGTKLTVL

SEQ ID NO: 56 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYANRWVFGGGTKLTVL

SEQ ID NO: 57 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVL

SEQ ID NO: 58 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNAWVFGGGTKLTVL

SEQ ID NO: 59 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRAVFGGGTKLTVL

SEQ ID NO: 60 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWAFGGGTKLTVL

SEQ ID NO: 61 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVL

SEQ ID NO: 62 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVL

SEQ ID NO: 63 - protein, 249 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVL

SEQ ID NO: 64 - protein, 6 aa, synthetic construct
QDVSIA

SEQ ID NO: 65 - skipped

SEQ ID NO: 66 - protein, 9 aa, synthetic construct
QQHYITPLT

SEQ ID NO: 67 - protein, 8 aa, synthetic construct
GYTFTNYG

SEQ ID NO: 68 - protein, 8 aa, synthetic construct
INTYTGEP

SEQ ID NO: 69 - protein, 14 aa, synthetic construct
ARGGFGSSYWYFDV

SEQ ID NO: 70 - protein, 14 aa, synthetic construct
ARGGFGSSYWYADV

SEQ ID NO: 71 - protein, 14 aa, synthetic construct
ARGGFGSSYWYFAV

SEQ ID NO: 72 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 74 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 75 - protein, 224 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 77 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCARHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 78 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVAHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 79 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 81 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGAFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 82 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 83 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFANSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 84 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGASYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 85 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 86 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSAISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 87 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYASYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYIAYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 89 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISAWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 90 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYAAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 91 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAAWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 92 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 93 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 94 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLAYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 95 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWASNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 96 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYANRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 97 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 98 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNAWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 99 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRAVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 100 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWAFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 101 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 102 - protein, 478 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 103 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 104 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 105 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 106 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 107 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 108 - protein, 478 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 109 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 110 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 111 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 112 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 113 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 114 - protein, 478 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFAVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 115 - protein, 6 aa, synthetic construct
QSIGTN

SEQ ID NO: 116 - skipped

SEQ ID NO: 117 - protein, 9 aa, synthetic construct
QQNNNWPTT

SEQ ID NO: 118 - protein, 8 aa, synthetic construct
GFSLTNYG

SEQ ID NO: 119 - protein, 7 aa, synthetic construct
IWSGGNT

SEQ ID NO: 120 - protein, 13 aa, synthetic construct
ARALTYYDYEFAY

SEQ ID NO: 121 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 123 - protein, 214 aa, synthetic construct; identical to SEQ ID NO: 121

SEQ ID NO: 124 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 125 - protein, 214 aa, synthetic construct; identical to SEQ ID NO: 121

SEQ ID NO: 126 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 127 - protein, 214 aa, synthetic construct; identical to SEQ ID NO: 121

SEQ ID NO: 128 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 129 - protein, 214 aa, synthetic construct; identical to SEQ ID NO: 121

SEQ ID NO: 130 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 131 - protein, 6 aa, synthetic construct
QGISNY

SEQ ID NO: 132 - skipped

SEQ ID NO: 133 - protein, 9 aa, synthetic construct
QNYNSAPFT

SEQ ID NO: 134 - protein, 8 aa, synthetic construct
GFAFSRYG

SEQ ID NO: 135 - protein, 8 aa, synthetic construct
IWYDGSNK

SEQ ID NO: 136 - protein, 16 aa, synthetic construct
ARGGDFLYYYYYGMDV

SEQ ID NO: 137 - protein, 214 aa, synthetic construct
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 226 aa, synthetic construct
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 139 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 226 aa, synthetic construct; identical to SEQ ID NO: 138

SEQ ID NO: 141 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 226 aa, synthetic construct; identical to SEQ ID NO: 138

SEQ ID NO: 143 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 226 aa, synthetic construct; identical to SEQ ID NO: 138

SEQ ID NO: 145 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 226 aa, synthetic construct; identical to SEQ ID NO: 138

SEQ ID NO: 147 - protein, 15 aa, synthetic construct
GGGGSGGGGSGGGGS

SEQ ID NO: 148 - protein, 5 aa, synthetic construct
GGGGS

SEQ ID NO: 149 - protein, 9 aa, synthetic construct
GGGGSGGGS

SEQ ID NO: 150 - protein, 28 aa, synthetic construct
GGGGSGGGLSGRSDAGSPLGLAGSGGGS

SEQ ID NO: 151 - protein, 18 aa, synthetic construct
GGGGSLSGRSDNHGSSGT

SEQ ID NO: 152 - protein, 26 aa, synthetic construct
GGGGSSGGSGGSGLSGRSDNHGSSGT

SEQ ID NO: 153 - protein, 8 aa, synthetic construct
ASGRSDNH

SEQ ID NO: 154 - protein, 8 aa, synthetic construct
LAGRSDNH

SEQ ID NO: 155 - protein, 13 aa, synthetic construct
ISSGLASGRSDNH

SEQ ID NO: 156 - protein, 13 aa, synthetic construct
ISSGLLAGRSDNH

SEQ ID NO: 157 - protein, 8 aa, synthetic construct
LSGRSDNH

SEQ ID NO: 158 - protein, 13 aa, synthetic construct
ISSGLLSGRSDNP

SEQ ID NO: 159 - protein, 13 aa, synthetic construct
ISSGLLSGRSDNH

SEQ ID NO: 160 - protein, 16 aa, synthetic construct
LSGRSDNHSPLGLAGS

SEQ ID NO: 161 - protein, 16 aa, synthetic construct
SPLGLAGSLSGRSDNH

SEQ ID NO: 162 - protein, 12 aa, synthetic construct
SPLGLSGRSDNH

SEQ ID NO: 163 - protein, 16 aa, synthetic construct
LAGRSDNHSPLGLAGS

SEQ ID NO: 164 - protein, 16 aa, synthetic construct
LSGRSDNHVPLSLKMG

SEQ ID NO: 165 - protein, 16 aa, synthetic construct
LSGRSDNHVPLSLSMG

SEQ ID NO: 166 - protein, 31 aa, synthetic construct
GSSGGSGGSGGSGISSGLLSGRSDNHGSSGT

SEQ ID NO: 167 - protein, 28 aa, synthetic construct
GSSGGSGGSGGISSGLLSGRSDNHGGGS

SEQ ID NO: 168 - protein, 8 aa, synthetic construct
ASGRSDNH

SEQ ID NO: 169 - protein, 8 aa, synthetic construct
LAGRSDNH

SEQ ID NO: 170 - protein, 13 aa, synthetic construct
ISSGLASGRSDNH

SEQ ID NO: 171 - protein, 8 aa, synthetic construct
LSGRSDAG

SEQ ID NO: 172 - protein, 13 aa, synthetic construct
ISSGLLSGRSDAG

SEQ ID NO: 173 - protein, 18 aa, synthetic construct
AAGLLAPPGGLSGRSDAG

SEQ ID NO: 174 - protein, 12 aa, synthetic construct
SPLGLSGRSDAG

SEQ ID NO: 175 - protein, 15 aa, synthetic construct
LSGRSDAGSPLGLAG

SEQ ID NO: 176 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 177 - protein, 478 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYADVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 178 - protein, 16 aa, synthetic construct
VRHGNFGNSYVSYWAY

(19) United States
(12) Patent Application Publication
(10) Pub. No.: US 2026/0049137 A1
(43) Pub. Date: Feb. 19, 2026

(54) OPTIMIZED ANTIBODIES TARGETING CD3 AND USES THEREOF

(71) Applicant: Janux Therapeutics, Inc., San Diego, CA (US)

(72) Inventors: David CAMPBELL, San Diego, CA (US); Thomas R. DiRaimondo, San Diego, CA (US)

(21) Appl. No.: 19/297,880

(22) Filed: Aug. 12, 2025

Related U.S. Application Data
(63) Continuation of application No. PCT/US24/16245, filed on Feb. 16, 2024.
(60) Provisional application No. 63/485,718, filed on Feb. 17, 2023.

(51) Int. Cl. C07K 16/28; A61K 39/00; A61P 35/00; C07K 16/30

(52) U.S. Cl. CPC C07K 16/2809; A61P 35/00; C07K 16/30; A61K 2039/505; C07K 2317/31; C07K 2317/622; C07K 2317/92; C07K 2317/94

(57) ABSTRACT

Disclosed herein are recombinant antibodies or antigen binding fragments thereof that comprise a CD3 binding domain that has been optimized via alanine scanning mutagenesis. In some embodiments, the recombinant antibodies or antigen binding fragments thereof further comprise an antigen binding domain.

### CROSS-REFERENCE

This application is a continuation of International Patent Application No. PCT/US2024/016245, filed Feb. 16, 2024, which claims the benefit of U.S. Provisional Application No. 63/485,718 filed on Feb. 17, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 15, 2024, is named 52426-751_301SL.xml and is 186,201 bytes in size.

### SUMMARY

Disclosed herein are recombinant antibodies or antigen binding fragments thereof that comprise a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2 (GTK), wherein CDR3-L comprises an amino acid sequence of X₁-X₂-W-X₃-X₄-X₅-X₆-W-X₇-X₈, wherein X₁ is V, G, P, L, I, M, S, T, or A, X₂ is L, G, P, V, I, M, S, T, or A, X₃ is Y, F, W, V, L, I, G, or A, X₄ is S, G, T, M, N, Q, H, or A, X₅ is N, Q, S, T, D, E, H, K, R, or A, X₆ is R, S, T, Q, D, E, H, K, N, or A, X₇ is V, G, P, L, I, M, S, T, or A, and X₈ is F, Y, W, V, L, I, G, or A, wherein CDR1-H comprises the amino acid sequence of SEQ ID NO: 15, wherein CDR2-H comprises the amino acid sequence of SEQ ID NO: 16, and wherein CDR3-H comprises the amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁, wherein X₉ is V, G, P, L, I, M, S, T, or A, X₁₀ is R, S, T, Q, D, E, H, K, N, or A, X₁₁ is H, R, K, G, T, S, N, Q, or A, X₁₂ is G, P, V, L, I, M, S, T, or A, X₁₃ is F, Y, W, V, L, I, G, or A, X₁₄ is G, P, V, L, I, M, S, T, or A, X₁₅ is N, Q, S, T, D, E, H, K, R, or A, X₁₆ is S, G, T, M, N, Q, H, or A, X₁₇ is Y, F, W, V, L, I, G, or A, X₁₈ is I, G, P, V, L, M, S, T, or A, X₁₉ is S, G, T, M, N, Q, H, or A, X₂₀ is W, F, Y, V, L, I, G, or A, and X₂₁ is Y, F, W, V, L, I, G, or A, and wherein when CDR3-L comprises the amino acid sequence of SEQ ID NO: 3 then CDR3-H does not comprise the amino acid sequence of SEQ ID NO: 17.

In some embodiments, X₁ is V, G, L, I, or A, X₂ is L, V, I, or A, X₃ is Y, W, F or A, X₄ is S, G, T, or A, X₅ is N, Q, D, E, or A, X₆ is R, K, or A, X₇ is V, G, L, I, or A, X₈ is F, Y, W, or A, X₉ is V, G, L, I, or A, X₁₀ is R, K, or A, X₁₂ is G, S, T, or A, X₁₃ is F, Y, W, or A, X₁₄ is G, S, T, or A, X₁₅ is N, Q, D, E, or A, X₁₆ is S, G, T, or A, X₁₇ is Y, W, F, or A, X₁₈ is I, V, L, or A, X₁₉ is S, G, T, or A, X₂₀ is W, Y, F, or A, and X₂₁ is Y, W, F, or A. In some embodiments, X₈ is F. In some embodiments, X₁₀ is R, X₁₁ is H, X₁₃ is F, X₁₈ is I, X₁₉ is S, and X₂₀ is W.

In some embodiments, CDR3-L comprises an amino acid sequence selected from SEQ ID NOs: 3, 5-6, 8-11, and 13-14. In some embodiments, the immunoglobulin light chain and the immunoglobulin heavy chain comprise a set of CDR sequences selected from the group consisting of CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; and CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17.

In some embodiments, the immunoglobulin light chain and the immunoglobulin heavy chain comprise a set of CDR sequences selected from the group consisting of CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; and CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17.

In some embodiments, CDR3-H comprises an amino acid sequence selected from SEQ ID NOs: 17, 19-22, 24-30, and 32-33. In some embodiments, the immunoglobulin light chain and the immunoglobulin heavy chain comprise a set of CDR sequences selected from the group consisting of CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

In some embodiments, the immunoglobulin light chain and the immunoglobulin heavy chain comprise a set of CDR sequences selected from the group consisting of CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

Further disclosed herein are recombinant antibodies or antigen binding fragments thereof that comprise a CD3 binding domain, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33; and CDR1-L: SEQ ID NO: 4, CDR2-L: SEQ ID NO: 2 (GTK), CDR3-L: SEQ ID NO: 5, and CDR1-H: SEQ ID NO: 18, CDR2-H: SEQ ID NO: 16, CDR3-H: SEQ ID NO: 178.

In some embodiments, the immunoglobulin light chain of the CD3 binding domain comprises a variable domain of an immunoglobulin kappa (IgK) or immunoglobulin lambda (IgL) light chain. In some embodiments, the immunoglobulin heavy chain of the CD3 binding domain comprises a variable domain of an IgG1, IgG2, IgG3, or IgG4 heavy chain. In some embodiments, the CD3 binding domain comprises a Fab, Fab′, (Fab′)₂, or a single chain variable fragment (scFv). In some embodiments, the CD3 binding domain is the scFv.

In some embodiments, the CD3 binding domain comprises an amino acid sequence with at least 90%, 95%, 96%, 97%, 98%, 99%, or 100% identity to SEQ ID NO: 37. In some embodiments, the CD3 binding domain comprises an amino acid sequence with at least 90%, 95%, 96%, 97%, 98%, 99%, or 100% identity to SEQ ID NO: 38. In some embodiments, the CD3 binding domain comprises an amino acid sequence with at least 90%, 95%, 96%, 97%, 98%, 99%, or 100% identity to SEQ ID NO: 39. In some embodiments, the CD3 binding domain comprises an amino acid sequence with at least 90%, 95%, 96%, 97%, 98%, 99%, or 100% identity to SEQ ID NO: 40. In some embodiments, the CD3 binding domain comprises an amino acid sequence with at least 90%, 95%, 96%, 97%, 98%, 99%, or 100% identity to SEQ ID NO: 41. In some embodiments, the CD3 binding domain comprises an amino acid sequence with at least 90%, 95%, 96%, 97%, 98%, 99%, or 100% identity to SEQ ID NO: 42. In some embodiments, the CD3 binding domain comprises an amino acid sequence with at least 90%, 95%, 96%, 97%, 98%, 99%, or 100% identity to SEQ ID NO: 43. In some embodiments, the CD3 binding domain comprises an amino acid sequence with at least 90%, 95%, 96%, 97%, 98%, 99%, or 100% identity to SEQ ID NO: 44. In some embodiments, the CD3 binding domain comprises an amino acid sequence with at least 90%, 95%, 96%, 97%, 98%, 99%, or 100% identity to SEQ ID NO: 45. In some embodiments, the CD3 binding domain comprises an amino acid sequence with at least 90%, 95%, 96%, 97%, 98%, 99%, or 100% identity to SEQ ID NO: 46. In some embodiments, the CD3 binding domain comprises an amino acid sequence with at least 90%, 95%, 96%, 97%, 98%, 99%, or 100% identity to SEQ ID NO: 47. In some embodiments, the CD3 binding domain comprises an amino acid sequence with at least 90%, 95%, 96%, 97%, 98%, 99%, or 100% identity to SEQ ID NO: 48. In some embodiments, the CD3 binding domain comprises an amino acid sequence with at least 90%, 95%, 96%, 97%, 98%, 99%, or 100% identity to SEQ ID NO: 49. In some embodiments, the CD3 binding domain comprises an amino acid sequence with at least 90%, 95%, 96%, 97%, 98%, 99%, or 100% identity to SEQ ID NO: 50. In some embodiments, the CD3 binding domain comprises an amino acid sequence with at least 90%, 95%, 96%, 97%, 98%, 99%, or 100% identity to SEQ ID NO: 51. In some embodiments, the CD3 binding domain comprises an amino acid sequence with at least 90%, 95%, 96%, 97%, 98%, 99%, or 100% identity to SEQ ID NO: 52. In some embodiments, the CD3 binding domain comprises an amino acid sequence with at least 90%, 95%, 96%, 97%, 98%, 99%, or 100% identity to SEQ ID NO: 53. In some embodiments, the CD3 binding domain comprises an amino acid sequence with at least 90%, 95%, 96%, 97%, 98%, 99%, or 100% identity to SEQ ID NO: 54. In some embodiments, the CD3 binding domain comprises an amino acid sequence with at least 90%, 95%, 96%, 97%, 98%, 99%, or 100% identity to SEQ ID NO: 55. In some embodiments, the CD3 binding domain comprises an amino acid sequence with at least 90%, 95%, 96%, 97%, 98%, 99%, or 100% identity to SEQ ID NO: 56. In some embodiments, the CD3 binding domain comprises an amino acid sequence with at least 90%, 95%, 96%, 97%, 98%, 99%, or 100% identity to SEQ ID NO: 57. In some embodiments, the CD3 binding domain comprises an amino acid sequence with at least 90%, 95%, 96%, 97%, 98%, 99%, or 100% identity to SEQ ID NO: 58. In some embodiments, the CD3 binding domain comprises an amino acid sequence with at least 90%, 95%, 96%, 97%, 98%, 99%, or 100% identity to SEQ ID NO: 59. In some embodiments, the CD3 binding domain comprises an amino acid sequence with at least 90%, 95%, 96%, 97%, 98%, 99%, or 100% identity to SEQ ID NO: 60. In some embodiments, the CD3 binding domain comprises an amino acid sequence with at least 90%, 95%, 96%, 97%, 98%, 99%, or 100% identity to SEQ ID NO: 61. In some embodiments, the CD3 binding domain comprises an amino acid sequence with at least 90%, 95%, 96%, 97%, 98%, 99%, or 100% identity to SEQ ID NO: 63.

In some embodiments, the recombinant antibody or antigen binding fragment thereof further comprises an antigen binding domain. In some embodiments, the antigen binding domain is selected from a tumor-associated calcium signal transducer 2 (TROP2) binding domain, an epidermal growth factor receptor (EGFR) binding domain, and a prostate-specific membrane antigen (PSMA) binding domain.

In some embodiments, the antigen binding domain is a TROP2 binding domain. In some embodiments, the TROP2 binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 64, CDR2-L comprises the amino acid sequence of SEQ ID NO: 65 (SAS), CDR3-L comprises the amino acid sequence of SEQ ID NO: 66, CDR1-H comprises the amino acid sequence of SEQ ID NO: 67, CDR2-H comprises the amino acid sequence of SEQ ID NO: 68, and CDR3-H comprises an amino acid sequence selected from any one of SEQ ID NOs: 69 and 70-71.

In some embodiments, the TROP2 binding domain comprises a Fab, Fab′, (Fab′)₂, or a single chain variable fragment (scFv). In some embodiments, the TROP2 binding domain is a Fab. In some embodiments, the immunoglobulin light chain of the TROP2 binding domain comprises a variable domain of an immunoglobulin kappa (IgK) or immunoglobulin lambda (IgL) light chain. In some embodiments, the immunoglobulin heavy chain of the TROP2 binding domain comprises a variable domain of an IgG1, IgG2, IgG3, or IgG4 heavy chain. In some embodiments, the N-term of the immunoglobulin heavy chain of the TROP2 binding domain is bound to the C-term of the immunoglobulin light chain of the CD3 binding domain. In some embodiments, the C-term of the immunoglobulin heavy chain of the TROP2 binding domain is bound to the N-term of the immunoglobulin light chain of the CD3 binding domain. In some embodiments, the N-term of the immunoglobulin heavy chain of the TROP2 binding domain is bound to the C-term of the immunoglobulin heavy chain of the CD3 binding domain. In some embodiments, the C-term of the immunoglobulin heavy chain of the TROP2 binding domain is bound to the N-term of the immunoglobulin heavy chain of the CD3 binding domain. In some embodiments, the N-term of the immunoglobulin light chain of the TROP2 binding domain is bound to the C-term of the immunoglobulin light chain of the CD3 binding domain. In some embodiments, the C-term of the immunoglobulin light chain of the TROP2 binding domain is bound to the N-term of the immunoglobulin light chain of the CD3 binding domain. In some embodiments, the N-term of the immunoglobulin light chain of the TROP2 binding domain is bound to the C-term of the immunoglobulin heavy chain of the CD3 binding domain. In some embodiments, the C-term of the immunoglobulin light chain of the TROP2 binding domain is bound to the N-term of the immunoglobulin heavy chain of the CD3 binding domain.

In some embodiments, the CD3 binding domain is a scFv and the TROP2 binding domain is a Fab or Fab′. In some embodiments, the scFv is bound to the immunoglobulin heavy chain of the Fab or Fab′. In some embodiments, the scFv is bound to the immunoglobulin light chain of the Fab or Fab′. In some embodiments, the immunoglobulin light chain of the scFv is bound to the immunoglobulin heavy chain of the Fab or Fab′. In some embodiments, the immunoglobulin light chain of the scFv is bound to the immunoglobulin light chain of the Fab or Fab′. In some embodiments, the immunoglobulin heavy chain of the scFv is bound to the immunoglobulin heavy chain of the Fab or Fab′. In some embodiments, the immunoglobulin heavy chain of the scFv is bound to the immunoglobulin light chain of the Fab or Fab′.

In some embodiments, the immunoglobulin light chain of the TROP2 binding domain comprises an amino acid sequence with at least 90%, 95%, 96%, 97%, 98%, 99%, or 100% identity to SEQ ID NO: 72. In some embodiments, the immunoglobulin heavy chain of the TROP2 binding domain comprises an amino acid sequence with at least 90%, 95%, 96%, 97%, 98%, 99%, or 100% identity to any one of SEQ ID NOs: 73-75. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 73. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 74. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 75.

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 77. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 78. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 79. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 80. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 81. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 82. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 83. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 84. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 85. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 86. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 87. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 88. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 89. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 90. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 91.

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 92. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 93. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 94. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 95. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 96. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 97. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 98. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 99. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 100. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 101.

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 105. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 106. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 107. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 111. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 112. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 113. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 109. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 176. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 177.

In some embodiments, the recombinant antibody or antigen binding fragment thereof comprises the amino acid sequences of SEQ ID NO: 72 and SEQ ID NO: 77. In some embodiments, the recombinant antibody or antigen binding fragment thereof comprises the amino acid sequences of SEQ ID NO: 72 and SEQ ID NO: 78. In some embodiments, the recombinant antibody or antigen binding fragment thereof comprises the amino acid sequences of SEQ ID NO: 72 and SEQ ID NO: 79. In some embodiments, the recombinant antibody or antigen binding fragment thereof comprises the amino acid sequences of SEQ ID NO: 72 and SEQ ID NO: 80. In some embodiments, the recombinant antibody or antigen binding fragment thereof comprises the amino acid sequences of SEQ ID NO: 72 and SEQ ID NO: 83. In some embodiments, the recombinant antibody or antigen binding fragment thereof comprises the amino acid sequences of SEQ ID NO: 72 and SEQ ID NO: 84. In some embodiments, the recombinant antibody or antigen binding fragment thereof comprises the amino acid sequences of SEQ ID NO: 72 and SEQ ID NO: 85. In some embodiments, the recombinant antibody or antigen binding fragment thereof comprises the amino acid sequences of SEQ ID NO: 72 and SEQ ID NO: 86. In some embodiments, the recombinant antibody or antigen binding fragment thereof comprises the amino acid sequences of SEQ ID NO: 72 and SEQ ID NO: 87. In some embodiments, the recombinant antibody or antigen binding fragment thereof comprises the amino acid sequences of SEQ ID NO: 72 and SEQ ID NO: 88. In some embodiments, the recombinant antibody or antigen binding fragment thereof comprises the amino acid sequences of SEQ ID NO: 72 and SEQ ID NO: 91.

In some embodiments, the recombinant antibody or antigen binding fragment thereof comprises the amino acid sequences of SEQ ID NO: 72 and SEQ ID NO: 92. In some embodiments, the recombinant antibody or antigen binding fragment thereof comprises the amino acid sequences of SEQ ID NO: 72 and SEQ ID NO: 93. In some embodiments, the recombinant antibody or antigen binding fragment thereof comprises the amino acid sequences of SEQ ID NO: 72 and SEQ ID NO: 95. In some embodiments, the recombinant antibody or antigen binding fragment thereof comprises the amino acid sequences of SEQ ID NO: 72 and SEQ ID NO: 96. In some embodiments, the recombinant antibody or antigen binding fragment thereof comprises the amino acid sequences of SEQ ID NO: 72 and SEQ ID NO: 97. In some embodiments, the recombinant antibody or antigen binding fragment thereof comprises the amino acid sequences of SEQ ID NO: 72 and SEQ ID NO: 98. In some embodiments, the recombinant antibody or antigen binding fragment thereof comprises the amino acid sequences of SEQ ID NO: 72 and SEQ ID NO: 100. In some embodiments, the recombinant antibody or antigen binding fragment thereof comprises the amino acid sequences of SEQ ID NO: 72 and SEQ ID NO: 101. In some embodiments, the recombinant antibody or antigen binding fragment thereof comprises the amino acid sequences of SEQ ID NO: 72 and SEQ ID NO: 105. In some embodiments, the recombinant antibody or antigen binding fragment thereof comprises the amino acid sequences of SEQ ID NO: 72 and SEQ ID NO: 109. In some embodiments, the recombinant antibody or antigen binding fragment thereof comprises the amino acid sequences of SEQ ID NO: 72 and SEQ ID NO: 176. In some embodiments, the recombinant antibody or antigen binding fragment thereof comprises the amino acid sequences of SEQ ID NO: 72 and SEQ ID NO: 177.

In some embodiments, the antigen binding domain of the recombinant antibody or antigen binding fragment thereof is an EGFR binding domain. In some embodiments, the EGFR binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 115, CDR2-L comprises the amino acid sequence of SEQ ID NO: 116 (YAS), CDR3-L comprises the amino acid sequence of SEQ ID NO: 117, CDR1-H comprises the amino acid sequence of SEQ ID NO: 118, CDR2-H comprises the amino acid sequence of SEQ ID NO: 119, and CDR3-H comprises the amino acid sequence of SEQ ID NO: 120.

In some embodiments, the immunoglobulin light chain of the EGFR binding domain comprises an amino acid sequence with at least 90%, 95%, 96%, 97%, 98%, 99%, or 100% identity to SEQ ID NO: 121, and the immunoglobulin heavy chain of the EGFR binding domain comprises an amino acid sequence with at least 90%, 95%, 96%, 97%, 98%, 99%, or 100% identity to SEQ ID NO: 122. In some embodiments, the recombinant antibody or antigen binding fragment thereof comprises amino acid sequences with at least 90%, 95%, 96%, 97%, 98%, 99%, or 100% identity to SEQ ID NO: 125 and SEQ ID NO: 126. In some embodiments, the recombinant antibody or antigen binding fragment thereof comprises amino acid sequences with at least 90%, 95%, 96%, 97%, 98%, 99%, or 100% identity to SEQ ID NO: 127 and SEQ ID NO: 128. In some embodiments, the recombinant antibody or antigen binding fragment thereof comprises amino acid sequences with at least 90%, 95%, 96%, 97%, 98%, 99%, or 100% identity to SEQ ID NO: 129 and SEQ ID NO: 130. In some embodiments, the recombinant antibody or antigen binding fragment thereof comprises the amino acid sequences of SEQ ID NO: 125 and SEQ ID NO: 126. In some embodiments, the recombinant antibody or antigen binding fragment thereof comprises the amino acid sequences of SEQ ID NO: 127 and SEQ ID NO: 128.

In some embodiments, the antigen binding domain of the recombinant antibody or antigen binding fragment thereof is a PSMA binding domain. In some embodiments, the PSMA binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 131, CDR2-L comprises the amino acid sequence of SEQ ID NO: 132 (EAS), CDR3-L comprises the amino acid sequence of SEQ ID NO: 133, CDR1-H comprises the amino acid sequence of SEQ ID NO: 134, CDR2-H comprises the amino acid sequence of SEQ ID NO: 135, and CDR3-H comprises the amino acid sequence of SEQ ID NO: 136. In some embodiments, the immunoglobulin light chain of the PSMA binding domain comprises an amino acid sequence with at least 90%, 95%, 96%, 97%, 98%, 99%, or 100% identity to SEQ ID NO: 137, and the immunoglobulin heavy chain of the PSMA binding domain comprises an amino acid sequence with at least 90%, 95%, 96%, 97%, 98%, 99%, or 100% identity to SEQ ID NO: 138.

In some embodiments, the recombinant antibody or antigen binding fragment thereof comprises amino acid sequences with at least 90%, 95%, 96%, 97%, 98%, 99%, or 100% identity to SEQ ID NO: 141 and SEQ ID NO: 142. In some embodiments, the recombinant antibody or antigen binding fragment thereof comprises amino acid sequences with at least 90%, 95%, 96%, 97%, 98%, 99%, or 100% identity to SEQ ID NO: 143 and SEQ ID NO: 144. In some embodiments, the recombinant antibody or antigen binding fragment thereof comprises amino acid sequences with at least 90%, 95%, 96%, 97%, 98%, 99%, or 100% identity to SEQ ID NO: 145 and SEQ ID NO: 146. In some embodiments, the recombinant antibody or antigen binding fragment thereof comprises the amino acid sequences of SEQ ID NO: 141 and SEQ ID NO: 142. In some embodiments, the recombinant antibody or antigen binding fragment thereof comprises the amino acid sequences of SEQ ID NO: 143 and SEQ ID NO: 144.

In some embodiments, the CD3 binding domain and the antigen binding domain are connected by a linker. In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 147. In some embodiments, the linker comprises the amino acid sequence of SEQ ID NO: 148. In some embodiments, the linker comprises the amino acid sequence of SEQ ID NO: 149. In some embodiments, the linker comprises an amino acid sequence selected from any one of SEQ ID NOs: 147-175.

Disclosed herein are pharmaceutical compositions comprising: (i) the recombinant antibody or antigen binding fragment thereof of any one of the embodiments disclosed herein; and (ii) a pharmaceutically acceptable excipient.

Disclosed herein are isolated recombinant nucleic acid molecules encoding a polypeptide of the recombinant antibody or antigen binding fragment thereof of any one of the embodiments disclosed herein.

Disclosed herein are vectors comprising an isolated nucleic acid encoding a polypeptide of the recombinant antibody or antigen binding fragment thereof of any one of the embodiments disclosed herein.

Disclosed herein are host cells comprising an isolated nucleic acid encoding a polypeptide of the recombinant antibody or antigen binding fragment thereof of any one of the embodiments disclosed herein.

Disclosed herein are methods of treating cancer in a subject in need thereof comprising administering to the subject the recombinant antibody or antigen binding fragment thereof of any one of the embodiments disclosed herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates binding curves and EC₅₀s for binding of human CD3 by TCE-1 to TCE-11 as measured by ELISA;

FIG. 2 illustrates binding curves and EC₅₀s for binding of human CD3 by TCE-1 and TCE-12 to TCE-21 as measured by ELISA;

FIG. 3 illustrates binding curves and EC₅₀s for binding of human CD3 by TCE-1 and TCE-22 to TCE-28 as measured by ELISA;

FIG. 4 illustrates binding curves and EC₅₀s for binding of CD3 binding by TCE-29 to TCE-34 as measured by ELISA;

FIGS. 5A-5B illustrate binding curves and EC₅₀s for binding of human CD3 (FIG. 5A) and cyno CD3 (FIG. 5B) by TCE-29, TCE-31, TCE-32, and TCE-34 as measured by ELISA;

FIG. 6 illustrates binding curves and EC₅₀s for binding of TROP2 by TCE-29, TCE-35, TCE-30, TCE-31, and TCE-32 as measured by ELISA;

FIG. 7 illustrates binding curves and EC₅₀s for binding of CD3 by TCE-29, TCE-35, TCE-30, TCE-31, and TCE-32 as measured by ELISA;

FIG. 8 illustrates binding curves and EC₅₀s for binding of TROP2 by TCE-34, TCE-37, TCE-38, and TCE-36 as measured by ELISA;

FIG. 9 illustrates binding curves and EC₅₀s for binding of CD3 by TCE-34, TCE-37, TCE-38, and TCE-36 as measured by ELISA;

FIGS. 10A-10O illustrate sensorgrams for TCE-2 to TCE-16 binding to CD3∈ as measured by biolayer interferometry (BLI);

FIGS. 11A-11N illustrate sensorgrams for TCE-17 to TCE-28 and TCE-1 binding to CD3∈ as measured by BLI;

FIGS. 12A-12D illustrate sensorgrams for TCE-3, TCE-4, TCE-7, and TCE-26 binding to CD3∈ as measured by BLI;

FIGS. 13A-13B illustrate sensorgrams for TCE-28 and TCE-1 binding to CD3∈ as measured by BLI;

FIGS. 14A-14B illustrate sensorgrams for TCE-29 binding to TROP2 (FIG. 14A) and CD3∈ (FIG. 14B) and calculated binding affinities (K_{D}), association rates (kₒₙ), and dissociation rates (k_{dis}) as measured by BLI;

FIGS. 15A-15B illustrate sensorgrams for TCE-30 (FIG. 15A) and TCE-31 (FIG. 15B) binding to TROP2 as measured by BLI;

FIGS. 16A-16B illustrate sensorgrams for TCE-30 (FIG. 16A) and TCE-31 (FIG. 16B) binding to CD3ε as measured by BLI;

FIGS. 17A-17B illustrate sensorgrams for TCE-32 binding to TROP2 (FIG. 17A) and CD3ε (FIG. 17B) as measured by BLI;

FIGS. 18A-18B illustrate sensorgrams for TCE-33 and TCE-34 binding to CD3ε as measured by BLI;

FIGS. 19A-19B illustrate sensorgrams for TCE-33 and TCE-34 binding to TROP2 as measured by BLI;

FIGS. 20A-20C illustrate sensorgrams for TCE-1, TCE-29, and TCE-35 binding to TROP2 as measured by BLI;

FIG. 21 illustrates killing of TROP2 positive NCI-H292 tumor cells by TCEs in the presence of CD8+ T-cells;

FIG. 22 illustrates killing of TROP2 positive NCI-H292 tumor cells by TCEs in the presence of CD8+ T-cells;

FIG. 23 illustrates killing of TROP2 positive NCI-H292 tumor cells by TCEs in the presence of CD8+ T-cells;

FIG. 24 illustrates killing of TROP2 positive HCT116 tumor cells by TCEs in the presence of CD8+ T-cells;

FIG. 25 illustrates killing of TROP2 positive NCI-H292 tumor cells by TCEs in the presence of CD8+ T-cells;

FIG. 26 illustrates killing of cynomolgus TROP2 positive HEK293 cells by TCEs in the presence of CD8+ T-cells;

FIG. 27 illustrates killing of TROP2 positive NCI-H292 tumor cells by TCEs in the presence of CD8+ T-cells;

FIG. 28 illustrates killing of TROP2 positive NCI-H292 tumor cells by a TCE in the presence of CD8+ T-cells;

FIG. 29 illustrates killing of cynomolgus TROP2 positive HEK293 cells by TCEs in the presence of CD8+ T-cells;

FIG. 30 illustrates killing of TROP2 positive NCI-H292 tumor cells by TCEs in the presence of CD8+ T-cells;

FIG. 31 illustrates killing of cynomolgus TROP2 positive HEK293 cells by TCEs in the presence of CD8+ T-cells;

FIG. 32 illustrates killing of TROP2 positive NCI-H292 tumor cells by TCEs in the presence of CD8+ T-cells;

FIG. 33 illustrates killing of cynomolgus TROP2 positive HEK293 cells by TCEs in the presence of CD8+ T-cells;

FIG. 34 illustrates results of pharmacokinetic and toxicity studies of TCE-35 in cynomolgus monkey;

FIG. 35 illustrates results of pharmacokinetic and toxicity studies of TCE-29 in cynomolgus monkey;

FIG. 36 shows cytokine release in cynomolgus monkey after continuous IV infusion of TCE-35;

FIG. 37 shows cytokine release in cynomolgus monkey after continuous IV infusion of TCE-29;

FIGS. 38A-38F show clinical chemistry parameters in cynomolgus monkeys after continuous IV infusion of TCE-35;

FIGS. 39A-39F show clinical chemistry parameters in cynomolgus monkeys after continuous IV infusion of TCE-29;

FIG. 40 illustrates pharmacokinetic and toxicity studies of TCE-31 in cynomolgus monkey;

FIG. 41 illustrates pharmacokinetic and toxicity studies of TCE-34 in cynomolgus monkey;

FIG. 42 illustrates pharmacokinetic and toxicity studies of TCE-32 in cynomolgus monkey;

FIGS. 43A-43D show cytokine release in cynomolgus monkeys after continuous IV infusion of TCE-31;

FIGS. 44A-44D show cytokine release in cynomolgus monkeys after continuous IV infusion of TCE-34;

FIG. 45 shows cytokine release in cynomolgus monkeys after continuous IV infusion of TCE-32;

FIGS. 46A-46F show clinical chemistry parameters in cynomolgus monkeys after continuous IV infusion of TCE-31;

FIGS. 47A-47F show clinical chemistry parameters in cynomolgus monkeys after continuous IV infusion of TCE-34; and

FIGS. 48A-48F show clinical chemistry parameters in cynomolgus monkeys after continuous IV infusion of TCE-32.

FIG. 49 illustrates binding curves and EC₅₀s for binding of TROP2 by TCE-48 and TCE-49 as measured by ELISA.

FIG. 50 illustrates binding curves and EC₅₀s for binding of CD3 by TCE-48 and TCE-49 as measured by ELISA.

FIG. 51 illustrates killing of TROP2 positive HCT116 tumor cells by TCE-48 and TCE-49 in the presence of CD8+ T-cells.

FIG. 52 illustrates killing of TROP2 positive H292 tumor cells by TCE-48 and TCE-49 in the presence of CD8+ T cells.

FIG. 53 illustrates killing of cynomolgus TROP2 positive HEK293 cells by TCE-48 and TCE-49 in the presence of CD8+ T-cells.

FIG. 54 illustrates binding curves and EC₅₀s for binding of EGFR by TCE-40 and TCE-42 as measured by ELISA.

FIG. 55 illustrates binding curves and EC₅₀s for binding of CD3 by TCE-40 and TCE-42 as measuring by ELISA.

FIG. 56 illustrates killing of EGFR positive HCT116 tumor cells by TCE-40 and TCE-42 in the presence of CD8+ T cells.

FIG. 57 illustrates killing of EGFR positive Cal27 cells by TCE-40 and TCE-42 in the presence of CD8+ T cells.

FIG. 58 illustrates binding curves and EC₅₀s for binding of PSMA by TCE-44 and TCE-46 as measured by ELISA.

FIG. 59 illustrates binding curves and EC₅₀s for binding of CD3 by TCE-44 and TCE-46 as measured by ELISA.

FIG. 60 illustrates killing of PSMA positive 22Rv1 tumor cells by TCE-44 and TCE-46 in the presence of CD8+ T cells.

FIG. 61 illustrates killing of PSMA positive LNCaP tumor cells by TCE-44 and TCE-46 in the presence of CD8+ T cells.

### DETAILED DESCRIPTION OF THE INVENTION

Cluster of differentiation 3 (CD3) is a protein complex which associates with the T cell receptor (TCR) during T cell activation. Because CD3 is required for T cell activation, drugs targeting CD3 may be candidates for immunosuppressant, autoimmune, and cancer therapies. Disclosed herein are recombinant antibodies or antigen binding fragments thereof that comprise a CD3 binding domain optimized through alanine scanning mutagenesis. Alanine scanning of the CD3 binding domain was accomplished by systematically mutating individual residues in the third complementarity determining regions (CDR3s) of the immunoglobulin light chain and the immunoglobulin heavy chain to alanine in order to establish CDR3 related structure activity relationships.

As disclosed herein, treatment of non-human primates with certain T-cell engager (TCE) constructs having single alanine mutations in the CD3 binding domain resulted in reduced levels of cytokine release compared to treatment with TCE constructs with a wild-type CD3 binding domain. Namely, the cytokine induction levels in non-human primates were lower for animals administered the maximum tolerated dose of CD3 mutant TCEs compared to animals administered with the maximum tolerated dose of wild-type CD3 TCEs. These results suggest reduced side effects related to cytokine release (e.g., cytokine release syndrome) after treatment with certain alanine mutated constructs. Additionally, the alanine mutated TCE constructs with weaker potencies may enable a wider therapeutic window between active dose levels and toxic dose levels relative to stronger potency wild-type constructs. This in turn could provide an advantage for standard dosing regimens where variability in population-based pharmacokinetics could be the difference between efficacy and toxicity. For example, controlling drug exposure is less challenging when there exists a wider drug exposure window between the maximum tolerated dose and the lethal dose. The difference in exposure between the non-human primate maximum tolerated dose and the lethal dose was larger for TCE constructs with reduced CD3 binding affinity and weaker in vitro cytotoxic potency. Further, the alanine mutated constructs with weaker potencies may also have benefits in analytics related to pharmacokinetic monitoring compared to stronger potency molecules which may be difficult to monitor at active doses when the detection limit of the analytical methods are above the drug exposure level. The weaker potency alanine mutated constructs disclosed herein may thus be easier to monitor clinically and advance in a clinical setting.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Phückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg NR and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Recombinant Antibody or Antigen Binding Fragment Compositions

### CD3 Binding Domain

Disclosed herein, in some embodiments, are recombinant antibodies or antigen binding fragments thereof that comprise a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2 (GTK), and wherein CDR3-L comprises an amino acid sequence of X₁-X₂-W-X₃-X₄-X₅-X₆-W-X₇-X₈ wherein X₁ is V, G, P, L, I, M, S, T, or A, X₂ is L, G, P, V, I, M, S, T, or A, X₃ is Y, F, W, V, L, I, G, or A, X₄ is S, G, T, M, N, Q, H, or A, X₅ is N, Q, S, T, D, E, H, K, R, or A, X₆ is R, S, T, Q, D, E, H, K, N, or A, X₇ is V, G, P, L, I, M, S, T, or A, and X₈ is F, Y, W, V, L, I, G, or A, wherein CDR1-H comprises the amino acid sequence of SEQ ID NO: 15, wherein CDR2-H comprises the amino acid sequence of SEQ ID NO: 16, wherein CDR3-H comprises the amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁, wherein X₉ is V, G, P, L, I, M, S, T, or A, X₁₀ is R, S, T, Q, D, E, H, K, N, or A, X₁₁ is H, R, K, G, T, S, N, Q, or A, X₁₂ is G, P, V, L, I, M, S, T, or A, X₁₃ is F, Y, W, V, L, I, G, or A, X₁₄ is G, P, V, L, I, M, S, T, or A, X₁₅ is N, Q, S, T, D, E, H, K, R, or A, X₁₆ is S, G, T, M, N, Q, H or A, X₁₇ is Y, F, W, V, L, I, G, or A, X₁₈ is I, G, P, V, L, M, S, T, or A, X₁₉ is S, G, T, M, N, Q, H, or A, X₂₀ is W, F, Y, V, L, I, G, or A, and X₂₁ is Y, F, W, V, L, I, G, or A, and wherein when CDR3-L comprises the sequence of SEQ ID NO: 3 then CDR3-H does not comprise the sequence of SEQ ID NO: 17 (see Tables 1-2).

In some embodiments, X₁ is V, G, L, I, or A, X₂ is L, V, I, or A, X₃ is Y, W, F or A, X₄ is S, G, T, or A, X₅ is N, Q, D, E, or A, X₆ is R, K, or A, X₇ is V, G, L, I, or A, X₈ is F, Y, W, or A, X₉ is V, G, L, I, or A, X₁₀ is R, K, or A, X₁₂ is G, S, T, or A, X₁₃ is F, Y, W or A, X₁₄ is G, S, T, or A, X₁₅ is N, Q, D, E or A, X₁₆ is S, G, T, or A, X₁₇ is Y, W, F, or A, X₁₈ is I, V, L, or A, X₁₉ is S, G, T, or A, X₂₀ is W, Y, F, or A, and X₂₁ is Y, W, F, or A.

In some embodiments, X₈ is F. In some embodiments, X₁₀ is R, X₁₁ is H, X₁₃ is F, X₁₈ is I, X₁₉ is S, and X₂₀ is W. In some embodiments, X₁₀ is R. In some embodiments, X₁₁ is H. In some embodiments, X₁₃ is F. In some embodiments, X₁₈ is I. In some embodiments, X₁₈ is I. In some embodiments, X₁₉ is S. In some embodiments, X₂₀ is W.

In some embodiments, CDR3-L comprises an amino acid sequence selected from SEQ ID NOs: 3, 5-6, 8-11, and 13-14 (see Table 1). In some embodiments, CDR3-L comprises the amino acid sequence of SEQ ID NO: 5. In some embodiments, CDR3-L comprises the amino acid sequence of SEQ ID NO: 6. In some embodiments, CDR3-L comprises the amino acid sequence of SEQ ID NO: 8. In some embodiments, CDR3-L comprises the amino acid sequence of SEQ ID NO: 9. In some embodiments, CDR3-L comprises the amino acid sequence of SEQ ID NO: 10. In some embodiments, CDR3-L comprises the amino acid sequence of SEQ ID NO: 11. In some embodiments, CDR3-L comprises the amino acid sequence of SEQ ID NO: 13. In some embodiments, CDR3-L comprises the amino acid sequence of SEQ ID NO: 14.

TABLE 1
Anti-CD3 scFv binding domain light chain
complementarity determining regions (CDRs)
as based on IMGT CDR numbering system.

| | Amino Acid | SEQ |
|---|---|---|
| Construct | Sequence | ID |
| Description | (N to C) | NO: |
| CDR1-L | TGAVTSGNY | 1 |
| CDR2-L | GTK | 2 |
| CDR3-L | VLWYSNRWV | 3 |
| CDR1-L | TGAVTSANY | 4 |
| CDR3-L V231A | ALWYSNRWV | 5 |
| CDR3-L L232A | VAWYSNRWV | 6 |
| CDR3-L W233A | VLAYSNRWV | 7 |
| CDR3-L Y234A | VLWASNRWV | 8 |
| CDR3-L S235A | VLWYANRWV | 9 |
| CDR3-L N236A | VLWYSARWV | 10 |
| CDR3-L R237A | VLWYSNAWV | 11 |
| CDR3-L W238A | VLWYSNRAV | 12 |
| CDR3-L V239A | VLWYSNRWA | 13 |
| CDR3-L F240A | VLWYSNRWVA | 14 |

TABLE 2
Anti-CD3 scFv binding domain heavy chain
complementarity determining regions (CDRs)
as based on IMGT CDR numbering system.

| Construct | Amino Acid Sequence | SEQ ID |
|---|---|---|
| Description | (N to C) | NO: |
| CDR1-H | GFTFNKYA | 15 |
| CDR2-H | IRSKYNNYAT | 16 |
| CDR3-H | VRHGNFGNSYISYWAY | 17 |
| CDR1-H | GFTFQKYA | 18 |
| CDR3-H | VRHGNFGNSYVSYWAY | 178 |
| CDR3-H V99A | ARHGNFGNSYISYWAY | 19 |
| CDR3-H R100A | VAHGNFGNSYISYWAY | 20 |
| CDR3-H H101A | VRAGNFGNSYISYWAY | 21 |
| CDR3-H G102A | VRHANFGNSYISYWAY | 22 |
| CDR3-H N103A | VRHGAFGNSYISYWAY | 23 |
| CDR3-H F104A | VRHGNAGNSYISYWAY | 24 |
| CDR3-H G105A | VRHGNFANSYISYWAY | 25 |
| CDR3-H N106A | VRHGNFGASYISYWAY | 26 |
| CDR3-H S107A | VRHGNFGNAYISYWAY | 27 |
| CDR3-H Y108A | VRHGNFGNSAISYWAY | 28 |
| CDR3-H I109A | VRHGNFGNSYASYWAY | 29 |
| CDR3-H S110A | VRHGNFGNSYIAYWAY | 30 |
| CDR3-H Y111A | VRHGNFGNSYISAWAY | 31 |
| CDR3-H W112A | VRHGNFGNSYISYAAY | 32 |
| CDR3-H Y114A | VRHGNFGNSYISYWAA | 33 |

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; and CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17.

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; and CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17.

In some embodiments, CDR3-H comprises an amino acid sequence selected from SEQ ID NOs: 17, 19-22, 24-30, and 32-33 (see Table 2). In some embodiments, CDR3-H comprises the amino acid sequence of SEQ ID NO: 19. In some embodiments, CDR3-H comprises the amino acid sequence of SEQ ID NO: 20. In some embodiments, CDR3-H comprises the amino acid sequence of SEQ ID NO: 21. In some embodiments, CDR3-H comprises the amino acid sequence of SEQ ID NO: 22. In some embodiments, CDR3-H comprises the amino acid sequence of SEQ ID NO: 24. In some embodiments, CDR3-H comprises the amino acid sequence of SEQ ID NO: 25. In some embodiments, CDR3-H comprises the amino acid sequence of SEQ ID NO: 26. In some embodiments, CDR3-H comprises the amino acid sequence of SEQ ID NO: 27. In some embodiments, CDR3-H comprises the amino acid sequence of SEQ ID NO: 28. In some embodiments, CDR3-H comprises the amino acid sequence of SEQ ID NO: 29. In some embodiments, CDR3-H comprises the amino acid sequence of SEQ ID NO: 30. In some embodiments, CDR3-H comprises the amino acid sequence of SEQ ID NO: 32. In some embodiments, CDR3-H comprises the amino acid sequence of SEQ ID NO: 33.

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

In some embodiments, the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

Disclosed herein, in some embodiments, are recombinant antibodies or antigen binding fragments thereof that comprise a CD3 binding domain that comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33; and CDR1-L: SEQ ID NO: 4, CDR2-L: SEQ ID NO: 2 (GTK), CDR3-L: SEQ ID NO: 5, and CDR1-H: SEQ ID NO: 18, CDR2-H: SEQ ID NO: 16, CDR3-H: SEQ ID NO: 178.

In some embodiments, the immunoglobulin light chain of the CD3 binding domain comprises a variable domain of an immunoglobulin kappa (IgK) or immunoglobulin lambda (IgL) light chain. In some embodiments, the immunoglobulin heavy chain of the CD3 binding domain comprises a variable domain of an IgG1, IgG2, IgG3, or IgG4 heavy chain. In some embodiments, the CD3 binding domain comprises a Fab, Fab′, (Fab′)₂, or a single chain variable fragment (scFv). In some embodiments, the CD3 binding domain is the scFv.

In some embodiments, the CD3 binding domain comprises an amino acid sequence with at least 90%, 95%, 96%, 97%, 98%, 99%, or 100% identity to SEQ ID NO: 37 (see Table 3). In some embodiments, the CD3 binding domain comprises an amino acid sequence with at least 90%, 95%, 96%, 97%, 98%, 99%, or 100% identity to SEQ ID NO: 38. In some embodiments, the CD3 binding domain comprises an amino acid sequence with at least 90%, 95%, 96%, 97%, 98%, 99%, or 100% identity to SEQ ID NO: 39. In some embodiments, the CD3 binding domain comprises an amino acid sequence with at least 90%, 95%, 96%, 97%, 98%, 99%, or 100% identity to SEQ ID NO: 40. In some embodiments, the CD3 binding domain comprises an amino acid sequence with at least 90%, 95%, 96%, 97%, 98%, 99%, or 100% identity to SEQ ID NO: 41. In some embodiments, the CD3 binding domain comprises an amino acid sequence with at least 90%, 95%, 96%, 97%, 98%, 99%, or 100% identity to SEQ ID NO: 42. In some embodiments, the CD3 binding domain comprises an amino acid sequence with at least 90%, 95%, 96%, 97%, 98%, 99%, or 100% identity to SEQ ID NO: 43. In some embodiments, the CD3 binding domain comprises an amino acid sequence with at least 90%, 95%, 96%, 97%, 98%, 99%, or 100% identity to SEQ ID NO: 44. In some embodiments, the CD3 binding domain comprises an amino acid sequence with at least 90%, 95%, 96%, 97%, 98%, 99%, or 100% identity to SEQ ID NO: 45. In some embodiments, the CD3 binding domain comprises an amino acid sequence with at least 90%, 95%, 96%, 97%, 98%, 99%, or 100% identity to SEQ ID NO: 46. In some embodiments, the CD3 binding domain comprises an amino acid sequence with at least 90%, 95%, 96%, 97%, 98%, 99%, or 100% identity to SEQ ID NO: 47. In some embodiments, the CD3 binding domain comprises an amino acid sequence with at least 90%, 95%, 96%, 97%, 98%, 99%, or 100% identity to SEQ ID NO: 48. In some embodiments, the CD3 binding domain comprises an amino acid sequence with at least 90%, 95%, 96%, 97%, 98%, 99%, or 100% identity to SEQ ID NO: 49. In some embodiments, the CD3 binding domain comprises an amino acid sequence with at least 90%, 95%, 96%, 97%, 98%, 99%, or 100% identity to SEQ ID NO: 50. In some embodiments, the CD3 binding domain comprises an amino acid sequence with at least 90%, 95%, 96%, 97%, 98%, 99%, or 100% identity to SEQ ID NO: 51. In some embodiments, the CD3 binding domain comprises an amino acid sequence with at least 90%, 95%, 96%, 97%, 98%, 99%, or 100% identity to SEQ ID NO: 52. In some embodiments, the CD3 binding domain comprises an amino acid sequence with at least 90%, 95%, 96%, 97%, 98%, 99%, or 100% identity to SEQ ID NO: 53. In some embodiments, the CD3 binding domain comprises an amino acid sequence with at least 90%, 95%, 96%, 97%, 98%, 99%, or 100% identity to SEQ ID NO: 54. In some embodiments, the CD3 binding domain comprises an amino acid sequence with at least 90%, 95%, 96%, 97%, 98%, 99%, or 100% identity to SEQ ID NO: 55. In some embodiments, the CD3 binding domain comprises an amino acid sequence with at least 90%, 95%, 96%, 97%, 98%, 99%, or 100% identity to SEQ ID NO: 56. In some embodiments, the CD3 binding domain comprises an amino acid sequence with at least 90%, 95%, 96%, 97%, 98%, 99%, or 100% identity to SEQ ID NO: 57. In some embodiments, the CD3 binding domain comprises an amino acid sequence with at least 90%, 95%, 96%, 97%, 98%, 99%, or 100% identity to SEQ ID NO: 58. In some embodiments, the CD3 binding domain comprises an amino acid sequence with at least 90%, 95%, 96%, 97%, 98%, 99%, or 100% identity to SEQ ID NO: 59. In some embodiments, the CD3 binding domain comprises an amino acid sequence with at least 90%, 95%, 96%, 97%, 98%, 99%, or 100% identity to SEQ ID NO: 60. In some embodiments, the CD3 binding domain comprises an amino acid sequence with at least 90%, 95%, 96%, 97%, 98%, 99%, or 100% identity to SEQ ID NO: 61. In some embodiments, the CD3 binding domain comprises an amino acid sequence with at least 90%, 95%, 96%, 97%, 98%, 99%, or 100% identity to SEQ ID NO: 62. In some embodiments, the CD3 binding domain comprises an amino acid sequence with at least 90%, 95%, 96%, 97%, 98%, 99%, or 100% identity to SEQ ID NO: 63.

TABLE 3
Anti-CD3 heavy chain and light chain sequences and scFvs

| | | SEQ |
|---|---|---|
| | | ID |
| Construct Description | Amino Acid Sequence (N to C) | NO: |
| CD3 light chain | QTVVTQEPSLTVSPGGTVTLTCGSSTGAVTSGNYPNWVQ | 34 |
| | QKPGQAPRGLIGGTKFLAPGTPARFSGSLLGGKAALTLS | |
| | GVQPEDEAEYYCVLWYSNRWVFGGGTKLTVL | |
| CD3 heavy chain | EVQLVESGGGLVQPGGSLKLSCAASGFTFNKYAMNWVR | 35 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSS | |
| CD3 scFv (SP34.185) wt | EVQLVESGGGLVQPGGSLKLSCAASGFTFNKYAMNWV | 36 |
| | RQAPGKGLEWVARIRSKYNNYATYYADSVKDRFTISRD | |
| | DSKNTAYLQMNNLKTEDTAVYYCVRHGNFGNSYISYW | |
| | AYWGQGTLVTVSSGGGGSGGGGSGGGGSQTVVTQEPSL | |
| | TVSPGGTVTLTCGSSTGAVTSGNYPNWVQQKPGQAPRG | |
| | LIGGTKFLAPGTPARFSGSLLGGKAALTLSGVQPEDEAEY | |
| | YCVLWYSNRWVFGGGTKLTVL | |
| CD3 scFv V99A | EVQLVESGGGLVQPGGSLKLSCAASGFTFNKYAMNWVR | 37 |
| | QAPGKGLEWVARIRSKYNNYATYYADSVKDRFTISRDDS | |
| | KNTAYLQMNNLKTEDTAVYYCARHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv R100A | EVQLVESGGGLVQPGGSLKLSCAASGFTFNKYAMNWVR | 38 |
| | QAPGKGLEWVARIRSKYNNYATYYADSVKDRFTISRDDS | |
| | KNTAYLQMNNLKTEDTAVYYCVAHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv H101A | EVQLVESGGGLVQPGGSLKLSCAASGFTFNKYAMNWVR | 39 |
| | QAPGKGLEWVARIRSKYNNYATYYADSVKDRFTISRDDS | |
| | KNTAYLQMNNLKTEDTAVYYCVRAGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv G102A | EVQLVESGGGLVQPGGSLKLSCAASGFTFNKYAMNWVR | 40 |
| | QAPGKGLEWVARIRSKYNNYATYYADSVKDRFTISRDDS | |
| | KNTAYLQMNNLKTEDTAVYYCVRHANFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv N103A | EVQLVESGGGLVQPGGSLKLSCAASGFTFNKYAMNWVR | 41 |
| | QAPGKGLEWVARIRSKYNNYATYYADSVKDRFTISRDDS | |
| | KNTAYLQMNNLKTEDTAVYYCVRHGAFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv F104A | EVQLVESGGGLVQPGGSLKLSCAASGFTFNKYAMNWVR | 42 |
| | QAPGKGLEWVARIRSKYNNYATYYADSVKDRFTISRDDS | |
| | KNTAYLQMNNLKTEDTAVYYCVRHGNAGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv G105A | EVQLVESGGGLVQPGGSLKLSCAASGFTFNKYAMNWVR | 43 |
| | QAPGKGLEWVARIRSKYNNYATYYADSVKDRFTISRDDS | |
| | KNTAYLQMNNLKTEDTAVYYCVRHGNFANSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv N106A | EVQLVESGGGLVQPGGSLKLSCAASGFTFNKYAMNWVR | 44 |
| | QAPGKGLEWVARIRSKYNNYATYYADSVKDRFTISRDDS | |
| | KNTAYLQMNNLKTEDTAVYYCVRHGNFGASYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv S107A | EVQLVESGGGLVQPGGSLKLSCAASGFTFNKYAMNWVR | 45 |
| | QAPGKGLEWVARIRSKYNNYATYYADSVKDRFTISRDDS | |
| | KNTAYLQMNNLKTEDTAVYYCVRHGNFGNAYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv Y108A | EVQLVESGGGLVQPGGSLKLSCAASGFTFNKYAMNWVR | 46 |
| | QAPGKGLEWVARIRSKYNNYATYYADSVKDRFTISRDDS | |
| | KNTAYLQMNNLKTEDTAVYYCVRHGNFGNSAISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv I109A | EVQLVESGGGLVQPGGSLKLSCAASGFTFNKYAMNWVR | 47 |
| | QAPGKGLEWVARIRSKYNNYATYYADSVKDRFTISRDDS | |
| | KNTAYLQMNNLKTEDTAVYYCVRHGNFGNSYASYWA | |
| | YWGQGTLVTVSSGGGGSGGGGSGGGGSQTVVTQEPSLT | |
| | VSPGGTVTLTCGSSTGAVTSGNYPNWVQQKPGQAPRGLI | |
| | GGTKFLAPGTPARFSGSLLGGKAALTLSGVQPEDEAEYY | |
| | CVLWYSNRWVFGGGTKLTVL | |
| CD3 scFv S110A | EVQLVESGGGLVQPGGSLKLSCAASGFTFNKYAMNWVR | 48 |
| | QAPGKGLEWVARIRSKYNNYATYYADSVKDRFTISRDDS | |
| | KNTAYLQMNNLKTEDTAVYYCVRHGNFGNSYIAYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv Y111A | EVQLVESGGGLVQPGGSLKLSCAASGFTFNKYAMNWVR | 49 |
| | QAPGKGLEWVARIRSKYNNYATYYADSVKDRFTISRDDS | |
| | KNTAYLQMNNLKTEDTAVYYCVRHGNFGNSYISAWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv W112A | EVQLVESGGGLVQPGGSLKLSCAASGFTFNKYAMNWVR | 50 |
| | QAPGKGLEWVARIRSKYNNYATYYADSVKDRFTISRDDS | |
| | KNTAYLQMNNLKTEDTAVYYCVRHGNFGNSYISYAAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv Y114A | EVQLVESGGGLVQPGGSLKLSCAASGFTFNKYAMNWVR | 51 |
| | QAPGKGLEWVARIRSKYNNYATYYADSVKDRFTISRDDS | |
| | KNTAYLQMNNLKTEDTAVYYCVRHGNFGNSYISYWAA | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVFGGGTKLTVL | |
| CD3 scFv V231A | EVQLVESGGGLVQPGGSLKLSCAASGFTFNKYAMNWVR | 52 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | ALWYSNRWVFGGGTKLTVL | |
| CD3 scFv L232A | EVQLVESGGGLVQPGGSLKLSCAASGFTFNKYAMNWVR | 53 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VAWYSNRWVFGGGTKLTVL | |
| CD3 scFv W233A | EVQLVESGGGLVQPGGSLKLSCAASGFTFNKYAMNWVR | 54 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLAYSNRWVFGGGTKLTVL | |
| CD3 scFv Y234A | EVQLVESGGGLVQPGGSLKLSCAASGFTFNKYAMNWVR | 55 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWASNRWVFGGGTKLTVL | |
| CD3 scFv S235A | EVQLVESGGGLVQPGGSLKLSCAASGFTFNKYAMNWVR | 56 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYANRWVFGGGTKLTVL | |
| CD3 scFv N236A | EVQLVESGGGLVQPGGSLKLSCAASGFTFNKYAMNWVR | 57 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSARWVFGGGTKLTVL | |
| CD3 scFv R237A | EVQLVESGGGLVQPGGSLKLSCAASGFTFNKYAMNWVR | 58 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNAWVFGGGTKLTVL | |
| CD3 scFv W238A | EVQLVESGGGLVQPGGSLKLSCAASGFTFNKYAMNWVR | 59 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRAVFGGGTKLTVL | |
| CD3 scFv V239A | EVQLVESGGGLVQPGGSLKLSCAASGFTFNKYAMNWVR | 60 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWAFGGGTKLTVL | |
| CD3 scFv F240A | EVQLVESGGGLVQPGGSLKLSCAASGFTFNKYAMNWVR | 61 |
| | QAPGKGLEWVARIRSKYNNYATYYADSVKDRFTISRDDS | |
| | KNTAYLQMNNLKTEDTAVYYCVRHGNFGNSYISYWAY | |
| | WGQGTLVTVSSGGGGSGGGGSGGGGSQTVVTQEPSLTV | |
| | SPGGTVTLTCGSSTGAVTSGNYPNWVQQKPGQAPRGLIG | |
| | GTKFLAPGTPARFSGSLLGGKAALTLSGVQPEDEAEYYC | |
| | VLWYSNRWVAGGGTKLTVL | |
| CD3 scFv | EVQLVESGGGLVQPGGSLKLSCAASGFTFNKYAMNWVR | 62 |
| P41S, A49G, N87S, L150F, | QASGKGLEWVGRIRSKYNNYATYYADSVKDRFTISRDDS | |
| T151S, G163R, P175A, | KNTAYLQMNSLKTEDTAVYYCVRHGNFGNSYISYWAY | |
| K181T, T200V, A202D, | WGQGTLVTVSSGGGGSGGGGSGGGGSQTVVTQEPSFSV | |
| L208I, G211N, L217I, | SPGGTVTLTCRSSTGAVTSGNYANWVQQTPGQAPRGLIG | |
| S218T, V220A, P222A, | GTKFLAPGVPDRFSGSILGNKAALTITGAQADDESDYYC | |
| E223D, A226S, E227D | VLWYSNRWVFGGGTKLTVL | |
| CD3 scFv | EVQLVESGGGLVQPGGSLKLSCAASGFTFQKYAMNWVR | 63 |
| N30Q, I109V, G172A, | QAPGKGLEWVARIRSKYNNYATYYADSVKDRFTISRDDS | |
| V231A | KNTAYLQMNNLKTEDTAVYYCVRHGNFGNSYVSYWAY | |
| | WGQGTLVTVSSGGGGSGGGGSGGGGSQTVVTQEPSLTV | |
| | SPGGTVTLTCGSSTGAVTSANYPNWVQQKPGQAPRGLIG | |
| | GTKFLAPGTPARFSGSLLGGKAALTLSGVQPEDEAEYYC | |
| | ALWYSNRWVFGGGTKLTVL | |

### Antigen Binding Domain

In some embodiments, the recombinant antibody or antigen binding fragment thereof further comprises an antigen binding domain. In some embodiments, the antigen binding domain is selected from a tumor-associated calcium signal transducer 2 (TROP2) binding domain, an epidermal growth factor receptor (EGFR) binding domain, and a prostate-specific membrane antigen (PSMA) binding domain.

### TROP2 Binding Domain

In some embodiments, the antigen binding domain is a TROP2 binding domain. In some embodiments, the TROP2 binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 64, CDR2-L comprises the amino acid sequence of SEQ ID NO: 65 (SAS), CDR3-L comprises the amino acid sequence of SEQ ID NO: 66, CDR1-H comprises the amino acid sequence of SEQ ID NO: 67, CDR2-H comprises the amino acid sequence of SEQ ID NO: 68, and CDR3-H comprises an amino acid sequence selected from any one of SEQ ID NOs: 69 and 70-71 (see Tables 4-5).

TABLE 4
TROP2 binding domain light chain complementarity
determining regions (CDRs) as based on
IMGT CDR numbering system.

| Construct | Amino Acid Sequence | SEQ ID |
|---|---|---|
| Description | (N to C) | NO: |
| CDR1-L | QDVSIA | 64 |
| CDR2-L | SAS | 65 |
| CDR3-L | QQHYITPLT | 66 |

TABLE 5
TROP2 binding domain heavy chain complementarity
determining regions (CDRs) as based on
IMGT CDR numbering system.

| Construct | Amino Acid Sequence | SEQ ID |
|---|---|---|
| Description | (N to C) | NO: |
| CDR1-H | GYTFTNYG | 67 |
| CDR2-H | INTYTGEP | 68 |
| CDR3-H | ARGGFGSSYWYFDV | 69 |
| CDR3-H F108A | ARGGFGSSYWYADV | 70 |
| CDR3-H D109A | ARGGFGSSYWYFAV | 71 |

In some embodiments, the TROP2 binding domain comprises a Fab, Fab′, (Fab′)₂, or a single chain variable fragment (scFv). In some embodiments, the TROP2 binding domain is a Fab. In some embodiments, the immunoglobulin light chain of the TROP2 binding domain comprises a variable domain of an immunoglobulin kappa (IgK) or immunoglobulin lambda (IgL) light chain. In some embodiments, the immunoglobulin heavy chain of the TROP2 binding domain comprises a variable domain of an IgG1, IgG2, IgG3, or IgG4 heavy chain.

In some embodiments, the N-term of the immunoglobulin heavy chain of the TROP2 binding domain is bound to the C-term of the immunoglobulin light chain of the CD3 binding domain. In some embodiments, the C-term of the immunoglobulin heavy chain of the TROP2 binding domain is bound to the N-term of the immunoglobulin light chain of the CD3 binding domain. In some embodiments, the N-term of the immunoglobulin heavy chain of the TROP2 binding domain is bound to the C-term of the immunoglobulin heavy chain of the CD3 binding domain. In some embodiments, the C-term of the immunoglobulin heavy chain of the TROP2 binding domain is bound to the N-term of the immunoglobulin heavy chain of the CD3 binding domain. In some embodiments, the N-term of the immunoglobulin light chain of the TROP2 binding domain is bound to the C-term of the immunoglobulin light chain of the CD3 binding domain. In some embodiments, the C-term of the immunoglobulin light chain of the TROP2 binding domain is bound to the N-term of the immunoglobulin light chain of the CD3 binding domain. In some embodiments, the N-term of the immunoglobulin light chain of the TROP2 binding domain is bound to the C-term of the immunoglobulin heavy chain of the CD3 binding domain. In some embodiments, the C-term of the immunoglobulin light chain of the TROP2 binding domain is bound to the N-term of the immunoglobulin heavy chain of the CD3 binding domain.

In some embodiments, the CD3 binding domain is a scFv and the TROP2 binding domain is a Fab or Fab′. In some embodiments, the scFv is bound to the immunoglobulin heavy chain of the Fab or Fab′. In some embodiments, the scFv is bound to the immunoglobulin light chain of the Fab or Fab′. In some embodiments, the immunoglobulin light chain of the scFv is bound to the immunoglobulin heavy chain of the Fab or Fab′. In some embodiments, the immunoglobulin light chain of the scFv is bound to the immunoglobulin light chain of the Fab or Fab′. In some embodiments, the immunoglobulin heavy chain of the scFv is bound to the immunoglobulin heavy chain of the Fab or Fab′. In some embodiments, the immunoglobulin heavy chain of the scFv is bound to the immunoglobulin light chain of the Fab or Fab′.

In some embodiments, the immunoglobulin light chain of the TROP2 binding domain comprises an amino acid sequence with at least 90%, 95%, 96%, 97%, 98%, 99%, or 100% identity to SEQ ID NO: 72 (see Table 6). In some embodiments, the immunoglobulin heavy chain of the TROP2 binding domain comprises an amino acid sequence with at least 90%, 95%, 96%, 97%, 98%, 99%, or 100% identity to any one of SEQ ID NOs: 73-75. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 73. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 74. In some embodiments, the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 75.

TABLE 6
TROP2 binding domain (TBD) heavy chain and light chain sequences

| Construct | | SEQ ID |
|---|---|---|
| Description | Amino Acid Sequence (N to C) | NO: |
| TBD-1 LC | DIQLTQSPSSLSASVGDRVSITCKASQDVSIAVAWYQQKPGK | 72 |
| | APKLLIYSASYRYTGVPDRFSGSGSGTDFTLTISSLQPEDFA | |
| | VYYCQQHYITPLTFGAGTKVEIKRTVAAPSVFIFPPSDEQLK | |
| | SGTASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-1 HC | QVQLQQSGSELKKPGASVKVSCKASGYTFTNYGMNWVKQ | 73 |
| | APGQGLKWMGWINTYTGEPTYTDDFKGRFAFSLDTSVSTA | |
| | YLQISSLKADDTAVYFCARGGFGSSYWYFDVWGQGSLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKKVEPKSC | |
| TBD-2 LC | DIQLTQSPSSLSASVGDRVSITCKASQDVSIAVAWYQQKPG | 72 |
| | KAPKLLIYSASYRYTGVPDRFSGSGSGTDFTLTISSLQPED | |
| | FAVYYCQQHYITPLTFGAGTKVEIKRTVAAPSVFIFPPSDE | |
| | QLKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| TBD-2 HC | QVQLQQSGSELKKPGASVKVSCKASGYTFTNYGMNWVKQ | 74 |
| F108A | APGQGLKWMGWINTYTGEPTYTDDFKGRFAFSLDTSVSTA | |
| | YLQISSLKADDTAVYFCARGGFGSSYWYADVWGQGSLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKKVEPKSC | |
| TBD-3 LC | DIQLTQSPSSLSASVGDRVSITCKASQDVSIAVAWYQQKPGK | 72 |
| | APKLLIYSASYRYTGVPDRFSGSGSGTDFTLTISSLQPEDFA | |
| | VYYCQQHYITPLTFGAGTKVEIKRTVAAPSVFIFPPSDEQLK | |
| | SGTASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| TBD-3 HC | QVQLQQSGSELKKPGASVKVSCKASGYTFTNYGMNWVKQ | 75 |
| D109A | APGQGLKWMGWINTYTGEPTYTDDFKGRFAFSLDTSVSTA | |
| | YLQISSLKADDTAVYFCARGGFGSSYWYFAVWGQGSLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKKVEPKSC | |

### TROP2 T Cell Engagers (TCEs)

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 77 (see Table 7). In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 78. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 79. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 80. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 81. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 82. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 83. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 84. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 85. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 86. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 87. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 88. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 89. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 90. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 91.

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 92. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 93. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 94. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 95. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 96. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 97. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 98. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 99. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 100. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 101. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 102. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 103. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 104.

In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 105. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 106. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 107. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 108. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 109. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 110. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 111. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 112. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 113. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 114. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 176. In some embodiments, the recombinant antibody or antigen binding fragment thereof comprises amino acid sequences with at least 90%, 95%, 96%, 97%, 98%, 99%, or 100% identity to SEQ ID NO: 72 and SEQ ID NO: 177.

In some embodiments, the recombinant antibody or antigen binding fragment thereof comprises the amino acid sequences of SEQ ID NO: 72 and SEQ ID NO: 77. In some embodiments, the recombinant antibody or antigen binding fragment thereof comprises the amino acid sequences of SEQ ID NO: 72 and SEQ ID NO: 78. In some embodiments, the recombinant antibody or antigen binding fragment thereof comprises the amino acid sequences of SEQ ID NO: 72 and SEQ ID NO: 79. In some embodiments, the recombinant antibody or antigen binding fragment thereof comprises the amino acid sequences of SEQ ID NO: 72 and SEQ ID NO: 80. In some embodiments, the recombinant antibody or antigen binding fragment thereof comprises the amino acid sequences of SEQ ID NO: 72 and SEQ ID NO: 83. In some embodiments, the recombinant antibody or antigen binding fragment thereof comprises the amino acid sequences of SEQ ID NO: 72 and SEQ ID NO: 84. In some embodiments, the recombinant antibody or antigen binding fragment thereof comprises the amino acid sequences of SEQ ID NO: 72 and SEQ ID NO: 85. In some embodiments, the recombinant antibody or antigen binding fragment thereof comprises the amino acid sequences of SEQ ID NO: 72 and SEQ ID NO: 86. In some embodiments, the recombinant antibody or antigen binding fragment thereof comprises the amino acid sequences of SEQ ID NO: 72 and SEQ ID NO: 87. In some embodiments, the recombinant antibody or antigen binding fragment thereof comprises the amino acid sequences of SEQ ID NO: 72 and SEQ ID NO: 88. In some embodiments, the recombinant antibody or antigen binding fragment thereof comprises the amino acid sequences of SEQ ID NO: 72 and SEQ ID NO: 91. In some embodiments, the recombinant antibody or antigen binding fragment thereof comprises the amino acid sequences of SEQ ID NO: 72 and SEQ ID NO: 92. In some embodiments, the recombinant antibody or antigen binding fragment thereof comprises the amino acid sequences of SEQ ID NO: 72 and SEQ ID NO: 93. In some embodiments, the recombinant antibody or antigen binding fragment thereof comprises the amino acid sequences of SEQ ID NO: 72 and SEQ ID NO: 95. In some embodiments, the recombinant antibody or antigen binding fragment thereof comprises the amino acid sequences of SEQ ID NO: 72 and SEQ ID NO: 96. In some embodiments, the recombinant antibody or antigen binding fragment thereof comprises the amino acid sequences of SEQ ID NO: 72 and SEQ ID NO: 97. In some embodiments, the recombinant antibody or antigen binding fragment thereof comprises the amino acid sequences of SEQ ID NO: 72 and SEQ ID NO: 98. In some embodiments, the recombinant antibody or antigen binding fragment thereof comprises the amino acid sequences of SEQ ID NO: 72 and SEQ ID NO: 100. In some embodiments, the recombinant antibody or antigen binding fragment thereof comprises the amino acid sequences of SEQ ID NO: 72 and SEQ ID NO: 101. In some embodiments, the recombinant antibody or antigen binding fragment thereof comprises the amino acid sequences of SEQ ID NO: 72 and SEQ ID NO: 105. In some embodiments, the recombinant antibody or antigen binding fragment thereof comprises the amino acid sequences of SEQ ID NO: 72 and SEQ ID NO: 109. In some embodiments, the recombinant antibody or antigen binding fragment thereof comprises the amino acid sequences of SEQ ID NO: 72 and SEQ ID NO: 176. In some embodiments, the recombinant antibody or antigen binding fragment thereof comprises the amino acid sequences of SEQ ID NO: 72 and SEQ ID NO: 177.

TABLE 7
TROP2 T cell engager (TCE) sequences

| Construct | | |
|---|---|---|
| Description | Amino Acid Sequence (N to C) | SEQ ID NO: |
| TCE-1 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-1 HC | EVQLVESGGGLVQPGGSLKLSCAASGFTFNKYAMNWVRQAP | 76 |
| anti-CD3 scFv wt | GKGLEWVARIRSKYNNYATYYADSVKDRFTISRDDSKNTAYL | |
| + linker + | QMNNLKTEDTAVYYCVRHGNFGNSYISYWAYWGQGTLVTV | |
| anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-2 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-2 HC | EVQLVESGGGLVQPGGSLKLSCAASGFTFNKYAMNWVRQAP | 77 |
| anti-CD3 scFv | GKGLEWVARIRSKYNNYATYYADSVKDRFTISRDDSKNTAYL | |
| (V99A) + linker + | QMNNLKTEDTAVYYCARHGNFGNSYISYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-3 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-3 HC | EVQLVESGGGLVQPGGSLKLSCAASGFTFNKYAMNWVRQAP | 78 |
| anti-CD3 scFv | GKGLEWVARIRSKYNNYATYYADSVKDRFTISRDDSKNTAYL | |
| (R100A) + linker | QMNNLKTEDTAVYYCVAHGNFGNSYISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-4 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-4 HC | EVQLVESGGGLVQPGGSLKLSCAASGFTFNKYAMNWVRQAP | 79 |
| anti-CD3 scFv | GKGLEWVARIRSKYNNYATYYADSVKDRFTISRDDSKNTAYL | |
| (H101A) + linker | QMNNLKTEDTAVYYCVRAGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-5 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-5 HC | EVQLVESGGGLVQPGGSLKLSCAASGFTFNKYAMNWVRQAP | 80 |
| anti-CD3 scFV | GKGLEWVARIRSKYNNYATYYADSVKDRFTISRDDSKNTAYL | |
| (G102A) + linker | QMNNLKTEDTAVYYCVRHANFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-6 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-6 HC | EVQLVESGGGLVQPGGSLKLSCAASGFTFNKYAMNWVRQAP | 81 |
| anti-CD3 scFv | GKGLEWVARIRSKYNNYATYYADSVKDRFTISRDDSKNTAYL | |
| (N103A) + linker | QMNNLKTEDTAVYYCVRHGAFGNSYISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-7 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-7 HC | EVQLVESGGGLVQPGGSLKLSCAASGFTFNKYAMNWVRQAP | 82 |
| anti-CD3 scFv | GKGLEWVARIRSKYNNYATYYADSVKDRFTISRDDSKNTAYL | |
| (F104A) + linker | QMNNLKTEDTAVYYCVRHGNAGNSYISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-8 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-8 HC | EVQLVESGGGLVQPGGSLKLSCAASGFTFNKY AMNWVRQAP | 83 |
| anti-CD3 scFv | GKGLEWVARIRSKYNNYATYYADSVKDRFTISRDDSKNTAYL | |
| (G105A) + linker | QMNNLKTEDTAVYYCVRHGNFANSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-9 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-9 HC | EVQLVESGGGLVQPGGSLKLSCAASGFTFNKYAMNWVRQAP | 84 |
| anti-CD3 scFv | GKGLEWVARIRSKYNNYATYYADSVKDRFTISRDDSKNTAYL | |
| (N106A) + linker | QMNNLKTEDTAVYYCVRHGNFGASYISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-10 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-10 HC | EVQLVESGGGLVQPGGSLKLSCAASGFTFNKYAMNWVRQAP | 85 |
| anti-CD3 scFv | GKGLEWVARIRSKYNNYATYYADSVKDRFTISRDDSKNTAYL | |
| (S107A) + linker | QMNNLKTEDTAVYYCVRHGNFGNAYISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-11 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-11 HC | EVQLVESGGGLVQPGGSLKLSCAASGFTFNKY AMNWVRQAP | 86 |
| anti-CD3 scFv | GKGLEWVARIRSKYNNYATYYADSVKDRFTISRDDSKNTAYL | |
| (Y108A) + linker | QMNNLKTEDTAVYYCVRHGNFGNSAISYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-12 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-12 HC | EVQLVESGGGLVQPGGSLKLSCAASGFTFNKYAMNWVRQAP | 87 |
| anti-CD3 scFv | GKGLEWVARIRSKYNNYATYYADSVKDRFTISRDDSKNTAYL | |
| (I109A) + linker + | QMNNLKTEDTAVYYCVRHGNFGNSYASYWAYWGQGTLVT | |
| anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-13 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-13 HC | EVQLVESGGGLVQPGGSLKLSCAASGFTFNKYAMNWVRQAP | 88 |
| anti-CD3 scFv | GKGLEWVARIRSKYNNYATYYADSVKDRFTISRDDSKNTAYL | |
| (S110A) + linker | QMNNLKTEDTAVYYCVRHGNFGNSYIAYWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-14 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-14 HC | EVQLVESGGGLVQPGGSLKLSCAASGFTFNKYAMNWVRQAP | 89 |
| anti-CD3 scFv | GKGLEWVARIRSKYNNYATYYADSVKDRFTISRDDSKNTAYL | |
| (Y111A) + linker | QMNNLKTEDTAVYYCVRHGNFGNSYISAWAYWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-15 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-15 HC | EVQLVESGGGLVQPGGSLKLSCAASGFTFNKYAMNWVRQAP | 90 |
| anti-CD3 scFv | GKGLEWVARIRSKYNNYATYYADSVKDRFTISRDDSKNTAYL | |
| (W112A) + linker | QMNNLKTEDTAVYYCVRHGNFGNSYISYAAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-16 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-16 HC | EVQLVESGGGLVQPGGSLKLSCAASGFTFNKYAMNWVRQAP | 91 |
| anti-CD3 scFv | GKGLEWVARIRSKYNNYATYYADSVKDRFTISRDDSKNTAYL | |
| (Y114A) + linker | QMNNLKTEDTAVYYCVRHGNFGNSYISYWAAWGQGTLVT | |
| + anti-TROP2 HC | VSSGGGGSGGGGSGGGGSQTVVTQEPSLTVSPGGTVTLTCGS | |
| wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-17 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-17 HC | EVQLVESGGGLVQPGGSLKLSCAASGFTFNKYAMNWVRQAP | 92 |
| anti-CD3 scFv | GKGLEWVARIRSKYNNYATYYADSVKDRFTISRDDSKNTAYL | |
| (V231A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCALWYSNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-18 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-18 HC | EVQLVESGGGLVQPGGSLKLSCAASGFTFNKY AMNWVRQAP | 93 |
| anti-CD3 scFv | GKGLEWVARIRSKYNNYATYYADSVKDRFTISRDDSKNTAYL | |
| (L232A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVAWYSNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-19 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-19 HC | EVQLVESGGGLVQPGGSLKLSCAASGFTFNKYAMNWVRQAP | 94 |
| anti-CD3 scFv | GKGLEWVARIRSKYNNYATYYADSVKDRFTISRDDSKNTAYL | |
| (W233A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLAYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-20 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-20 HC | EVQLVESGGGLVQPGGSLKLSCAASGFTFNKYAMNWVRQAP | 95 |
| anti-CD3 scFv | GKGLEWVARIRSKYNNYATYYADSVKDRFTISRDDSKNTAYL | |
| (Y234A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWASNRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-21 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-21 HC | EVQLVESGGGLVQPGGSLKLSCAASGFTFNKYAMNWVRQAP | 96 |
| anti-CD3 scFv | GKGLEWVARIRSKYNNYATYYADSVKDRFTISRDDSKNTAYL | |
| (S235A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYANRWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-22 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-22 HC | EVQLVESGGGLVQPGGSLKLSCAASGFTFNKYAMNWVRQAP | 97 |
| anti-CD3 scFv | GKGLEWVARIRSKYNNYATYYADSVKDRFTISRDDSKNTAYL | |
| (N236A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSARWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-23 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-23 HC | EVQLVESGGGLVQPGGSLKLSCAASGFTFNKYAMNWVRQAP | 98 |
| anti-CD3 scFv | GKGLEWVARIRSKYNNYATYYADSVKDRFTISRDDSKNTAYL | |
| (R237A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNAWVFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-24 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-24 HC | EVQLVESGGGLVQPGGSLKLSCAASGFTFNKYAMNWVRQAP | 99 |
| anti-CD3 scFv | GKGLEWVARIRSKYNNYATYYADSVKDRFTISRDDSKNTAYL | |
| (W238A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRAVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-25 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-25 HC | EVQLVESGGGLVQPGGSLKLSCAASGFTFNKYAMNWVRQAP | 100 |
| anti-CD3 scFv | GKGLEWVARIRSKYNNYATYYADSVKDRFTISRDDSKNTAYL | |
| (V239A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWAFGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-26 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-26 HC | EVQLVESGGGLVQPGGSLKLSCAASGFTFNKYAMNWVRQAP | 101 |
| anti-CD3 scFv | GKGLEWVARIRSKYNNYATYYADSVKDRFTISRDDSKNTAYL | |
| (F240A) + linker | QMNNLKTEDTAVYYCVRHGNFGNSYISYWAYWGQGTLVTV | |
| + anti-TROP2 HC | SSGGGGSGGGGSGGGGSQTVVTQEPSLTVSPGGTVTLTCGSST | |
| wt | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFDVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-27 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-27 HC | EVQLVESGGGLVQPGGSLKLSCAASGFTFNKYAMNWVRQAS | 102 |
| anti-CD3 scFv + | GKGLEWVGRIRSKYNNYATYYADSVKDRFTISRDDSKNTAYL | |
| linker + anti- | QMNSLKTEDTAVYYCVRHGNFGNSYISYWAYWGQGTLVTV | |
| TROP2 HC wt | SSGGGGSGGGGSGGGGSQTVVTQEPSFSVSPGGTVTLTCRSST | |
| | GAVTSGNYANWVQQTPGQAPRGLIGGTKFLAPGVPDRFSGSI | |
| | LGNKAALTITGAQADDESDYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFDVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-28 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-28 HC | EVQLVESGGGLVQPGGSLKLSCAASGFTFQKYAMNWVRQAP | 103 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 HC wt | VSSGGGGSGGGGSGGGGSQTVVTQEPSLTVSPGGTVTLTCGS | |
| | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFDVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-29 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-29 HC | EVQLVESGGGLVQPGGSLKLSCAASGFTFNKYAMNWVRQAP | 104 |
| anti-CD3 scFv wt | GKGLEWVARIRSKYNNYATYYADSVKDRFTISRDDSKNTAYL | |
| + linker + anti- | QMNNLKTEDTAVYYCVRHGNFGNSYISYWAYWGQGTLVTV | |
| TROP2 (F108A) | SSGGGGSGGGGSGGGGSQTVVTQEPSLTVSPGGTVTLTCGSST | |
| HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-30 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-30 HC | EVQLVESGGGLVQPGGSLKLSCAASGFTFNKY AMNWVRQAP | 105 |
| anti-CD3 scFv | GKGLEWVARIRSKYNNYATYYADSVKDRFTISRDDSKNTAYL | |
| (H101A) + linker | QMNNLKTEDTAVYYCVAAGNFGNSYISYWAYWGQGTLVTV | |
| + anti-TROP2 | SSGGGGSGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-31 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-31 HC | EVQLVESGGGLVQPGGSLKLSCAASGFTFNKYAMNWVRQAP | 106 |
| anti-CD3 scFv | GKGLEWVARIRSKYNNYATYYADSVKDRFTISRDDSKNTAYL | |
| (F104A) + linker | QMNNLKTEDTAVYYCVRHGNAGNSYISYWAYWGQGTLVT | |
| + anti-TROP2 | VSSGGGGSGGGGSGGGGSQTVVTQEPSLTVSPGGTVTLTCGS | |
| (F108A) HC | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYADVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-32 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-32 HC | EVQLVESGGGLVQPGGSLKLSCAASGFTFNKYAMNWVRQAP | 107 |
| anti-CD3 scFv | GKGLEWVARIRSKYNNYATYYADSVKDRFTISRDDSKNTAYL | |
| (F240A) + linker | QMNNLKTEDTAVYYCVRHGNFGNSYISYWAYWGQGTLVTV | |
| + anti-TROP2 | SSGGGGSGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYADVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-33 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-33 HC | EVQLVESGGGLVQPGGSLKLSCAASGFTFNKYAMNWVRQAS | 108 |
| anti-CD3 scFv + | GKGLEWVGRIRSKYNNYATYYADSVKDRFTISRDDSKNTAYL | |
| linker + anti- | QMNSLKTEDTAVYYCVRHGNFGNSYISYWAYWGQGTLVTV | |
| TROP2 (F108A) | SSGGGGSGGGGSGGGGSQTVVTQEPSFSVSPGGTVTLTCRSST | |
| HC | GAVTSGNYANWVQQTPGQAPRGLIGGTKFLAPGVPDRFSGSI | |
| | LGNKAALTITGAQADDESDYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-34 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-34 HC | EVQLVESGGGLVQPGGSLKLSCAASGFTFQKYAMNWVRQAP | 109 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 (F108A) | VSSGGGGSGGGGSGGGGSQTVVTQEPSLTVSPGGTVTLTCGS | |
| HC | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYADVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-35 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-35 HC | EVQLVESGGGLVQPGGSLKLSCAASGFTFNKYAMNWVRQAP | 110 |
| anti-CD3 scFv wt | GKGLEWVARIRSKYNNYATYYADSVKDRFTISRDDSKNTAYL | |
| + linker + anti- | QMNNLKTEDTAVYYCVRHGNFGNSYISYWAYWGQGTLVTV | |
| TROP2 (D109A) | SSGGGGSGGGGSGGGGSQTVVTQEPSLTVSPGGTVTLTCGSST | |
| HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFAVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-36 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-36 HC | EVQLVESGGGLVQPGGSLKLSCAASGFTFNKYAMNWVRQAP | 111 |
| anti-CD3 scFv | GKGLEWVARIRSKYNNYATYYADSVKDRFTISRDDSKNTAYL | |
| (H101A) + linker | QMNNLKTEDTAVYYCVRAGNFGNSYISYWAYWGQGTLVTV | |
| + anti-TROP2 | SSGGGGSGGGGSGGGGSQTVVTQEPSLTVSPGGTVTLTCGSST | |
| (D109A) HC | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYFAVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-37 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-37 HC | EVQLVESGGGLVQPGGSLKLSCAASGFTFNKY AMNWVRQAP | 112 |
| anti-CD3 scFv | GKGLEWVARIRSKYNNYATYYADSVKDRFTISRDDSKNTAYL | |
| (F104A) + linker | QMNNLKTEDTAVYYCVRHGNAGNSYISYWAYWGQGTLVT | |
| + anti-TROP2 | VSSGGGGSGGGGSGGGGSQTVVTQEPSLTVSPGGTVTLTCGS | |
| (D109A) HC | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFAVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-38 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-38 HC | EVQLVESGGGLVQPGGSLKLSCAASGFTFNKYAMNWVRQAP | 113 |
| anti-CD3 scFv | GKGLEWVARIRSKYNNYATYYADSVKDRFTISRDDSKNTAYL | |
| (F240A) + linker | QMNNLKTEDTAVYYCVRHGNFGNSYISYWAYWGQGTLVTV | |
| + anti-TROP2 | SSGGGGSGGGGSGGGGSQTVVTQEPSLTVSPGGTVTLTCGSST | |
| (D109A) HC | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLQQSGSELKKPGASVKVSCKASGYTFTNYGMN | |
| | WVKQAPGQGLKWMGWINTYTGEPTYTDDFKGRFAFSLDTSV | |
| | STAYLQISSLKADDTAVYFCARGGFGSSYWYFAVWGQGSLV | |
| | TVSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-39 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLOPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-39 HC | EVQLVESGGGLVQPGGSLKLSCAASGFTFQKYAMNWVRQAP | 114 |
| anti-CD3 scFv + | GKGLEWVARIRSKYNNYATYYADSVKDRFTISRDDSKNTAYL | |
| linker + anti- | QMNNLKTEDTAVYYCVRHGNFGNSYVSYWAYWGQGTLVT | |
| TROP2 (D109A) | VSSGGGGSGGGGSGGGGSQTVVTQEPSLTVSPGGTVTLTCGS | |
| HC | STGAVTSANYPNWVQQKPGQAPRGLIGGTKFLAPGTPARFSG | |
| | SLLGGKAALTLSGVQPEDEAEYYCALWYSNRWVFGGGTKLT | |
| | VLGGGGSQVQLQQSGSELKKPGASVKVSCKASGYTFTNYGM | |
| | NWVKQAPGQGLKWMGWINTYTGEPTYTDDFKGRFAFSLDTS | |
| | VSTAYLQISSLKADDTAVYFCARGGFGSSYWYFAVWGQGSL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQTY | |
| | ICNVNHKPSNTKVDKKVEPKSC | |
| TCE-48 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-48 HC | EVQLVESGGGLVQPGGSLKLSCAASGFTFNKYAMNWVRQAP | 176 |
| anti-CD3 scFv | GKGLEWVARIRSKYNNYATYYADSVKDRFTISRDDSKNTAYL | |
| (L232A) + linker | QMNNLKTEDTAVYYCVRHGNFGNSYISYWAYWGQGTLVTV | |
| + anti-TROP2 | SSGGGGSGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVAWYSNRWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |
| TCE-49 LC | DIQLTQSPSSLSASVGDRVSITCKASQDVSIAVAWYQQKPGKA | 72 |
| anti-TROP2 LC | PKLLIYSASYRYTGVPDRFSGSGSGTDFTLTISSLQPEDFAVYY | |
| wt | CQQHYITPLTFGAGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| TCE-49 HC | EVQLVESGGGLVQPGGSLKLSCAASGFTFNKYAMNWVRQAP | 177 |
| anti-CD3 scFv | GKGLEWVARIRSKYNNYATYYADSVKDRFTISRDDSKNTAYL | |
| (N236A) + linker | QMNNLKTEDTAVYYCVRHGNFGNSYISYWAYWGQGTLVTV | |
| + anti-TROP2 | SSGGGGGGGGSGGGGSQTVVTQEPSLTVSPGGTVTLTCGSST | |
| (F108A) HC | GAVTSGNYPNWVQQKPGQAPRGLIGGTKFLAPGTPARFSGSL | |
| | LGGKAALTLSGVQPEDEAEYYCVLWYSARWVFGGGTKLTVL | |
| | GGGGSQVQLQQSGSELKKPGASVKVSCKASGYTFTNYGMNW | |
| | VKQAPGQGLKWMGWINTYTGEPTYTDDFKGRFAFSLDTSVS | |
| | TAYLQISSLKADDTAVYFCARGGFGSSYWYADVWGQGSLVT | |
| | VSSASTKGPSVFPLAPSSKSTSGGTAALGCLVKDYFPEPVTVS | |
| | WNSGALTSGVHTFPAVLQSSGLYSLSSVVTVPSSSLGTQTYIC | |
| | NVNHKPSNTKVDKKVEPKSC | |

### EGFR Binding Domain

In some embodiments, the antigen binding domain of the recombinant antibody or antigen binding fragment thereof is an EGFR binding domain. In some embodiments, the EGFR binding domain comprises an immunoglobulin light chain comprising CDRs: CDR-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 115, CDR2-L comprises the amino acid sequence of SEQ ID NO: 116 (YAS), CDR3-L comprises the amino acid sequence of SEQ ID NO: 117, CDR1-H comprises the amino acid sequence of SEQ ID NO: 118, CDR2-H comprises the amino acid sequence of SEQ ID NO: 119, and CDR3-H comprises the amino acid sequence of SEQ ID NO: 120 (see Tables 8-9).

TABLE 8
EGFR binging domain light chain complementarity
determining regions (CDRs) as based on
IMGT CDR numbering system.

| Construct | Amino Acid | SEQ ID |
|---|---|---|
| Description | Sequence (N to C) | NO: |
| CDR1-L | QSIGTN | 115 |
| CDR2-L | YAS | 116 |
| CDR3-L | QQNNNWPTT | 117 |

TABLE 9
EGFR binding domain heavy chain complementarity
determining regions (CDRs) as based on
IMGT CDR numbering system.

| Construct | Amino Acid | SEQ ID |
|---|---|---|
| Description | Sequence (N to C) | NO: |
| CDR1-H | GFSLTNYG | 118 |
| CDR2-H | IWSGGNT | 119 |
| CDR3-H | ARALTYYDYEFAY | 120 |

In some embodiments, the immunoglobulin light chain of the EGFR binding domain comprises an amino acid sequence with at least 90%, 95%, 96%, 97%, 98%, 99%, or 100% identity to SEQ ID NO: 121, and the immunoglobulin heavy chain of the EGFR binding domain comprises an amino acid sequence with at least 90%, 95%, 96%, 97%, 98%, 99%, or 100% identity to SEQ ID NO: 122 (see Table 10).

TABLE 10
EGFR binding domain (EGFR BD) heavy and light chain sequences

| Construct | | SEQ ID |
|---|---|---|
| Description | Amino Acid Sequence (N to C) | NO: |
| EGFR BD-1 LC | DILLTQSPVILSVSPGERVSFSCRASQSIGTNIHWYQQRTNGSP | 121 |
| | RLLIKYASESISGIPSRFSGSGSGTDFTLSINSVESEDIADYYC | |
| | QQNNNWPTTFGAGTKLELKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| EGFR BD-2 HC | QVQLKQSGPGLVQPSQSLSITCTVSGFSLTNYGVHWVRQSPG | 122 |
| | KGLEWLGVIWSGGNTDYNTPFTSRLSINKDNSKSQVFFKMNS | |
| | LQSQDTAIYYCARALTYYDYEFAYWGQGTLVTVSAASTKGPS | |
| | VFPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSC | |

### EGFR TCEs

In some embodiments, the recombinant antibody or antigen binding fragment thereof comprises amino acid sequences with at least 90%, 95%, 96%, 97%, 98%, 99%, or 100% identity to SEQ ID NO: 125 and SEQ ID NO: 126 (see Table 11). In some embodiments, the recombinant antibody or antigen binding fragment thereof comprises amino acid sequences with at least 90%, 95%, 96%, 97%, 98%, 99%, or 100% identity to SEQ ID NO: 127 and SEQ ID NO: 128. In some embodiments, the recombinant antibody or antigen binding fragment thereof comprises amino acid sequences with at least 90%, 95%, 96%, 97%, 98%, 99%, or 100% identity to SEQ ID NO: 129 and SEQ ID NO: 130. In some embodiments, the recombinant antibody or antigen binding fragment thereof comprises the amino acid sequences of SEQ ID NO: 125 and SEQ ID NO: 126. In some embodiments, the recombinant antibody or antigen binding fragment thereof comprises the amino acid sequences of SEQ ID NO: 127 and SEQ ID NO: 128.

TABLE 11
EGFR T cell engager (TCE) sequences

| Construct | | SEQ ID |
|---|---|---|
| Description | Amino Acid Sequence (N to C) | NO: |
| TCE-40 LC | DILLTQSPVILSVSPGERVSFSCRASQSIGTNIHWYQQRTNGSP | 123 |
| anti-EFGR LC wt | RLLIKYASESISGIPSRFSGSGSGTDFTLSINSVESEDIADYYCQQ | |
| | NNNWPTTFGAGTKLELKRTVAAPSVFIFPPSDEQLKSGTASVV | |
| | CLLNNFYPREAKVQWKVDNALQSGNSQESVTEQDSKDSTYS | |
| | LSSTLTLSKADYEKHKVYACEVTHQGLSSPVTKSFNRGEC | |
| TCE-40 HC | EVQLVESGGGLVQPGGSLKLSCAASGFTFNKYAMNWVRQAP | 124 |
| anti-CD3 scFv wt | GKGLEWVARIRSKYNNYATYYADSVKDRFTISRDDSKNTAYL | |
| + linker + | QMNNLKTEDTAVYYCVRHGNFGNSYISYWAYWGQGTLVTV | |
| anti-EGFR HC wt | SSGGGGSGGGGSGGGGSQTVVTQEPSLTVSPGGTVTLTCGSST | |
| | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLKQSGPGLVQPSQSLSITCTVSGFSLTNYGVHWV | |
| | RQSPGKGLEWLGVIWSGGNTDYNTPFTSRLSINKDNSKSQVFF | |
| | KMNSLQSQDTAIYYCARALTYYDYEFAYWGQGTLVTVSAAS | |
| | TKGPSVFPLAPSSKSTSGGTAALGCLVKDYFPEPVTVSWNSGA | |
| | LTSGVHTFPAVLQSSGLYSLSSVVTVPSSSLGTQTYICNVNHKP | |
| | SNTKVDKKVEPKSC | |
| TCE-41 LC | DILLTQSPVILSVSPGERVSFSCRASQSIGTNIHWYQQRTNGSP | 125 |
| anti-EFGR LC wt | RLLIKYASESISGIPSRFSGSGSGTDFTLSINSVESEDIADYYC | |
| | QQNNNWPTTFGAGTKLELKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| TCE-41 HC | EVQLVESGGGLVQPGGSLKLSCAASGFTFNKYAMNWVRQAP | 126 |
| anti-CD3 scFv | GKGLEWVARIRSKYNNYATYYADSVKDRFTISRDDSKNTAYL | |
| (H101A) + linker | QMNNLKTEDTAVYYCVRAGNFGNSYISYWAYWGQGTLVTV | |
| + | SSGGGGSGGGGSGGGGSQTVVTQEPSLTVSPGGTVTLTCGSST | |
| anti-EGFR HC wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSQVQLKQSGPGLVQPSQSLSITCTVSGFSLTNYGVHWV | |
| | RQSPGKGLEWLGVIWSGGNTDYNTPFTSRLSINKDNSKSQVFF | |
| | KMNSLQSQDTAIYYCARALTYYDYEFAYWGQGTLVTVSAAS | |
| | TKGPSVFPLAPSSKSTSGGTAALGCLVKDYFPEPVTVSWNSGA | |
| | LTSGVHTFPAVLQSSGLYSLSSVVTVPSSSLGTQTYICNVNHKP | |
| | SNTKVDKKVEPKSC | |
| TCE-42 LC | DILLTQSPVILSVSPGERVSFSCRASQSIGTNIHWYQQRTNGSP | 127 |
| anti-EFGR LC wt | RLLIKYASESISGIPSRFSGSGSGTDFTLSINSVESEDIADYYC | |
| | QQNNNWPTTFGAGTKLELKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| TCE-42 HC | EVQLVESGGGLVQPGGSLKLSCAASGFTFNKYAMNWVRQAP | 128 |
| anti-CD3 scFv | GKGLEWVARIRSKYNNYATYYADSVKDRFTISRDDSKNTAYL | |
| (F104A) + linker | QMNNLKTEDTAVYYCVRHGNAGNSYISYWAYWGQGTLVT | |
| 1 | VSSGGGGSGGGGSGGGGSQTVVTQEPSLTVSPGGTVTLTCGS | |
| anti-EGFR HC wt | STGAVTSGNYPNWVQQKPGQAPRGLIGGTKFLAPGTPARFSG | |
| | SLLGGKAALTLSGVQPEDEAEYYCVLWYSNRWVFGGGTKLT | |
| | VLGGGGSQVQLKQSGPGLVQPSQSLSITCTVSGFSLTNYGVH | |
| | WVRQSPGKGLEWLGVIWSGGNTDYNTPFTSRLSINKDNSKSQ | |
| | VFFKMNSLQSQDTAIYYCARALTYYDYEFAYWGQGTLVTVS | |
| | AASTKGPSVFPLAPSSKSTSGGTAALGCLVKDYFPEPVTVSWN | |
| | SGALTSGVHTFPAVLQSSGLYSLSSVVTVPSSSLGTQTYICNVN | |
| | HKPSNTKVDKKVEPKSC | |
| TCE-43 LC | DILLTQSPVILSVSPGERVSFSCRASQSIGTNIHWYQQRTNGSP | 129 |
| anti-EFGR LC wt | RLLIKYASESISGIPSRFSGSGSGTDFTLSINSVESEDIADYYC | |
| | QQNNNWPTTFGAGTKLELKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| TCE-43 HC | EVQLVESGGGLVQPGGSLKLSCAASGFTFNKYAMNWVRQAP | 130 |
| anti-CD3 scFv | GKGLEWVARIRSKYNNYATYYADSVKDRFTISRDDSKNTAYL | |
| (F240A) + linker | QMNNLKTEDTAVYYCVRHGNFGNSYISYWAYWGQGTLVTV | |
| + anti-EGFR | SSGGGGSGGGGSGGGGSQTVVTQEPSLTVSPGGTVTLTCGSST | |
| HC wt | GAVTSGNYPNWVQQKPGQAPRGLIGGTKFLAPGTPARFSGSL | |
| | LGGKAALTLSGVQPEDEAEYYCVLWYSNRWVAGGGTKLTV | |
| | LGGGGSQVQLKQSGPGLVQPSQSLSITCTVSGFSLTNYGVHW | |
| | VRQSPGKGLEWLGVIWSGGNTDYNTPFTSRLSINKDNSKSQV | |
| | FFKMNSLQSQDTAIYYCARALTYYDYEFAYWGQGTLVTVSA | |
| | ASTKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKVEPKSC | |

### PSMA Binding Domain

In some embodiments, the antigen binding domain of the recombinant antibody or antigen binding fragment thereof is a PSMA binding domain. In some embodiments, the PSMA binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 131, CDR2-L comprises the amino acid sequence of SEQ ID NO: 132 (EAS), CDR3-L comprises the amino acid sequence of SEQ ID NO: 133, CDR1-H comprises the amino acid sequence of SEQ ID NO: 134, CDR2-H comprises the amino acid sequence of SEQ ID NO: 135, and CDR3-H comprises the amino acid sequence of SEQ ID NO: 136 (see Tables 12-13).

TABLE 12
PSMA binding domain light chain complementarity
determining regions (CDRs)

| Construct | Amino Acid | SEQ ID |
|---|---|---|
| Description | Sequence (N to C) | NO: |
| CDR1-L | QGISNY | 131 |
| CDR2-L | EAS | 132 |
| CDR3-L | QNYNSAPFT | 133 |

TABLE 13
PSMA binding domain heavy chain complementarity
determining regions (CDRs)

| Construct | Amino Acid | SEQ ID |
|---|---|---|
| Description | Sequence (N to C) | NO: |
| CDR1-H | GFAFSRYG | 134 |
| CDR2-H | IWYDGSNK | 135 |
| CDR3-H | ARGGDFLYYYYYGMDV | 136 |

In some embodiments, the immunoglobulin light chain of the PSMA binding domain comprises an amino acid sequence with at least 90%, 95%, 96%, 97%, 98%, 99%, or 100% identity to SEQ ID NO: 137, and the immunoglobulin heavy chain of the PSMA binding domain comprises an amino acid sequence with at least 90%, 95%, 96%, 97%, 98%, 99%, or 100% identity to SEQ ID NO: 138 (see Table 14).

TABLE 14
PSMA binding domain (PSMA BD) heavy and light chain sequences

| Construct | Amino Acid | SEQ ID |
|---|---|---|
| Description | Sequence (N to C) | NO: |
| PSMA BD-1 LC | DIQMTQSPSSLSASVGDRVTITCRASQGISNYLAWYQQKTGK | 137 |
| | VPKFLIYEASTLQSGVPSRFSGGGSGTDFTLTISSLQPEDVA | |
| | TYYCQNYNSAPFTFGPGTKVDIKRTVAAPSVFIFPPSDEQLK | |
| | SGTASVVCLLNNFYPREAKVQWKVDNALQSGNSQESVTEQDS | |
| | KDSTYSLSSTLTLSKADYEKHKVYACEVTHQGLSSPVTKSFN | |
| | RGEC | |
| PSMA BD-2 HC | QVQLVESGGGVVQPGRSLRLSCAASGFAFSRYGMHWVRQAP | 138 |
| | GKGLEWVAVIWYDGSNKYYADSVKGRFTISRDNSKNTQYLQ | |
| | MNSLRAEDTAVYYCARGGDFLYYYYYGMDVWGQGTTVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSC | |

### PSMA TCEs

In some embodiments, the recombinant antibody or antigen binding fragment thereof comprises amino acid sequences with at least 90%, 95%, 96%, 97%, 98%, 99%, or 100% identity to SEQ ID NO: 141 and SEQ ID NO: 142 (see Table 15). In some embodiments, the recombinant antibody or antigen binding fragment thereof comprises amino acid sequences with at least 90%, 95%, 96%, 97%, 98%, 99%, or 100% identity to SEQ ID NO: 143 and SEQ ID NO: 144. In some embodiments, the recombinant antibody or antigen binding fragment thereof comprises amino acid sequences with at least 90%, 95%, 96%, 97%, 98%, 99%, or 100% identity to SEQ ID NO: 145 and SEQ ID NO: 146. In some embodiments, the recombinant antibody or antigen binding fragment thereof comprises the amino acid sequences of SEQ ID NO: 141 and SEQ ID NO: 142. In some embodiments, the recombinant antibody or antigen binding fragment thereof comprises the amino acid sequences of SEQ ID NO: 143 and SEQ ID NO: 144.

TABLE 15
PSMA T Amino Acid Sequence (N to C)

| Construct | engager (TCE) sequences | SEQ ID |
|---|---|---|
| Description | Amino Acid Sequence (N to C) | NO: |
| TCE-44 LC | EVQLVESGGGLVQPGGSLKLSCAASGFTFNKYAMNWVRQAP | 139 |
| anti-CD3 scFv wt | GKGLEWVARIRSKYNNYATYYADSVKDRFTISRDDSKNTAYL | |
| + linker + | QMNNLKTEDTAVYYCVRHGNFGNSYISYWAYWGQGTLVTV | |
| anti-PSMA LC wt | SSGGGGSGGGGSGGGGSQTVVTQEPSLTVSPGGTVTLTCGSST | |
| | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSDIQMTQSPSSLSASVGDRVTITCRASQGISNYLAWYQQ | |
| | KTGKVPKFLIYEASTLQSGVPSRFSGGGSGTDFTLTISSLQPED | |
| | VATYYCQNYNSAPFTFGPGTKVDIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| TCE-44 HC | QVQLVESGGGVVQPGRSLRLSCAASGFAFSRYGMHWVRQAP | 140 |
| anti-PSMA HC wt | GKGLEWVAVIWYDGSNKYYADSVKGRFTISRDNSKNTQYLQ | |
| | MNSLRAEDTAVYYCARGGDFLYYYYYGMDVWGQGTTVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICN | |
| | VNHKPSNTKVDKKVEPKSC | |
| TCE-45 LC | EVQLVESGGGLVQPGGSLKLSCAASGFTFNKYAMNWVRQAP | 141 |
| anti-CD3 scFv | GKGLEWVARIRSKYNNYATYYADSVKDRFTISRDDSKNTAYL | |
| (H101A) + linker | QMNNLKTEDTAVYYCVRAGNFGNSYISYWAYWGQGTLVTV | |
| + | SSGGGGSGGGGSGGGGSQTVVTQEPSLTVSPGGTVTLTCGSST | |
| anti-PSMA LC wt | GAVTSGNYPNWVQQKPGQAPRGLIGGTKFLAPGTPARFSGSL | |
| | LGGKAALTLSGVQPEDEAEYYCVLWYSNRWVFGGGTKLTVL | |
| | GGGGSDIQMTQSPSSLSASVGDRVTITCRASQGISNYLAWYQQ | |
| | KTGKVPKFLIYEASTLQSGVPSRFSGGGSGTDFTLTISSLQPED | |
| | VATYYCQNYNSAPFTFGPGTKVDIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| TCE-45 HC | QVQLVESGGGVVQPGRSLRLSCAASGFAFSRYGMHWVRQAP | 142 |
| anti-PSMA HC wt | GKGLEWVAVIWYDGSNKYYADSVKGRFTISRDNSKNTQYLQ | |
| | MNSLRAEDTAVYYCARGGDFLYYYYYGMDVWGQGTTVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSC | |
| TCE-46 LC | EVQLVESGGGLVQPGGSLKLSCAASGFTFNKYAMNWVRQAP | 143 |
| anti-CD3 scFv | GKGLEWVARIRSKYNNYATYYADSVKDRFTISRDDSKNTAYL | |
| (F104A) + linker | QMNNLKTEDTAVYYCVRHGNAGNSYISYWAYWGQGTLVT | |
| + | VSSGGGGSGGGGSGGGGSQTVVTQEPSLTVSPGGTVTLTCGS | |
| anti-PSMA LC wt | STGAVTSGNYPNWVQQKPGQAPRGLIGGTKFLAPGTPARFSG | |
| | SLLGGKAALTLSGVQPEDEAEYYCVLWYSNRWVFGGGTKLTV | |
| | LGGGGSDIQMTQSPSSLSASVGDRVTITCRASQGISNYLAWY | |
| | QQKTGKVPKFLIYEASTLQSGVPSRFSGGGSGTDFTLTISSL | |
| | QPEDVATYYCQNYNSAPFTFGPGTKVDIKRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |
| TCE-46 HC | QVQLVESGGGVVQPGRSLRLSCAASGFAFSRYGMHWVRQAP | 144 |
| anti-PSMA HC wt | GKGLEWVAVIWYDGSNKYYADSVKGRFTISRDNSKNTQYLQ | |
| | MNSLRAEDTAVYYCARGGDFLYYYYYGMDVWGQGTTVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSC | |
| TCE-47 LC | EVQLVESGGGLVQPGGSLKLSCAASGFTFNKYAMNWVRQAP | 145 |
| anti-CD3 scFv | GKGLEWVARIRSKYNNYATYYADSVKDRFTISRDDSKNTAYL | |
| (F240A) + linker | QMNNLKTEDTAVYYCVRHGNFGNSYISYWAYWGQGTLVTVS | |
| + | SGGGGSGGGGSGGGGSQTVVTQEPSLTVSPGGTVTLTCGSST | |
| anti-PSMA LC wt | GAVTSGNYPNWVQQKPGQAPRGLIGGTKFLAPGTPARFSGSL | |
| | LGGKAALTLSGVQPEDEAEYYCVLWYSNRWVAGGGTKLTVLG | |
| | GGGSDIQMTQSPSSLSASVGDRVTITCRASQGISNYLAWYQQ | |
| | KTGKVPKFLIYEASTLQSGVPSRFSGGGSGTDFTLTISSLQP | |
| | EDVATYYCQNYNSAPFTFGPGTKVDIKRTVAAPSVFIFPPSD | |
| | EQLKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| TCE-47 HC | QVQLVESGGGVVQPGRSLRLSCAASGFAFSRYGMHWVRQAP | 146 |
| anti-PSMA HC wt | GKGLEWVAVIWYDGSNKYYADSVKGRFTISRDNSKNTQYLQ | |
| | MNSLRAEDTAVYYCARGGDFLYYYYYGMDVWGQGTTVTVSS | |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSC | |

### Linker Sequences

In some embodiments, the CD3 binding domain and the antigen binding domain are connected by a linker. In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 147 (see Table 16). In some embodiments, the linker comprises the amino acid sequence of SEQ ID NO: 148. In some embodiments, the linker comprises the amino acid sequence of SEQ ID NO: 149. In some embodiments, the linker comprises an amino acid sequence selected from any one of SEQ ID NOs: 147-175.

TABLE 16
Linker Sequences

| Construct | Amino Acid Sequence | SEQ ID |
|---|---|---|
| Description | (N to C) | NO: |
| Linker 1 | GGGGSGGGGSGGGGS | 147 |
| Linker 2 | GGGGS | 148 |
| Linker 3 | GGGGSGGGS | 149 |
| Cleavable | GGGGSGGGLSGRSDA | 150 |
| linker | GSPLGLAGSGGGS | |
| Linker 4 | GGGGSLSGRSDNHGSSGT | 151 |
| Linker 5 | GGGGSSGGSGGSGLS | 152 |
| | GRSDNHGSSGT | |
| Linker 6 | ASGRSDNH | 153 |
| Linker 7 | LAGRSDNH | 154 |
| Linker 8 | ISSGLASGRSDNH | 155 |
| Linker 9 | ISSGLLAGRSDNH | 156 |
| Linker 10 | LSGRSDNH | 157 |
| Linker 11 | ISSGLLSGRSDNP | 158 |
| Linker 12 | ISSGLLSGRSDNH | 159 |
| Linker 13 | LSGRSDNHSPLGLAGS | 160 |
| Linker 14 | SPLGLAGSLSGRSDNH | 161 |
| Linker 15 | SPLGLSGRSDNH | 162 |
| Linker 16 | LAGRSDNHSPLGLAGS | 163 |
| Linker 17 | LSGRSDNHVPLSLKMG | 164 |
| Linker 18 | LSGRSDNHVPLSLSMG | 165 |
| Linker 19 | GSSGGSGGSGGSGISS | 166 |
| | GLLSGRSDNHGSSGT | |
| Linker 20 | GSSGGSGGSGGISSGL | 167 |
| | LSGRSDNHGGGS | |
| Linker 21 | ASGRSDNH | 168 |
| Linker 22 | LAGRSDNH | 169 |
| Linker 23 | ISSGLASGRSDNH | 170 |
| Linker 24 | LSGRSDAG | 171 |
| Linker 25 | ISSGLLSGRSDAG | 172 |
| Linker 26 | AAGLLAPPGGLSGRS | 173 |
| | DAG | |
| Linker 27 | SPLGLSGRSDAG | 174 |
| Linker 28 | LSGRSDAGSPLGLAG | 175 |

### Modified Amino Acids

In some embodiments, the recombinant antibody or antigen binding fragment thereof comprises a modified amino acid, a non-natural amino acid, a modified non-natural amino acid, or a combination thereof. In some embodiments, the non-natural amino acid comprises a D-amino acid. In some embodiments, the modified amino acid or modified non-natural amino acid comprises a post-translational modification.

### Pharmaceutical Compositions

Disclosed herein, in some embodiments, are pharmaceutical compositions comprising: (i) recombinant antibodies or antigen binding fragments thereof according to any embodiment disclosed herein; and (ii) a pharmaceutically acceptable excipient.

Disclosed herein, in some embodiments, are pharmaceutical compositions comprising a recombinant antibody or antigen binding fragment thereof that comprises a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2 (GTK), and wherein CDR3-L comprises an amino acid sequence of X₁-X₂-W-X₃-X₄-X₅-X₆-W-X₇-X₈ wherein X₁ is V, G, P, L, I, M, S, T, or A, X₂ is L, G, P, V, I, M, S, T, or A, X₃ is Y, F, W, V, L, I, G, or A, X₄ is S, G, T, M, N, Q, H or A, X₅ is N, Q, S, T, D, E, H, K, R, or A, X₆ is R, S, T, Q, D, E, H, K, N, or A, X₇ is V, G, P, L, I, M, S, T, or A, and X₈ is F, Y, W, V, L, I, G, or A, wherein CDR1-H comprises the amino acid sequence of SEQ ID NO: 15, wherein CDR2-H comprises the amino acid sequence of SEQ ID NO: 16, wherein CDR3-H comprises the amino acid sequence of X₉-X₁₀-X₁₁-X₁₂-N-X₁₃-X₁₄-X₁₅-X₁₆-X₁₇-X₁s-X₁₉-Y-X₂₀-A-X₂₁, wherein X₉ is V, G, P, L, I, M, S, T, or A, X₁₀ is R, S, T, Q, D, E, H, K, N, or A, X₁₁ is H, R, K, G, T, S, N, Q, or A, X₁₂ is G, P, V, L, I, M, S, T, or A, X₁₃ is F, Y, W, V, L, I, G, or A, X₁₄ is G, P, V, L, I, M, S, T, or A, X₁₅ is N, Q, S, T, D, E, H, K, R, or A, X₁₆ is S, G, T, M, N, Q, H, or A, X₁₇ is Y, F, W, V, L, I, G, or A, X₁₈ is I, G, P, V, L, M, S, T, or A, X₁₉ is S, G, T, M, N, Q, H, or A, X₂₀ is W, F, Y, V, L, I, G, or A, and X₂₁ is Y, F, W, V, L, I, G, or A, wherein when CDR3-L comprises the sequence of SEQ ID NO: 3 then CDR3-H does not comprise the sequence of SEQ ID NO: 17.

Disclosed herein, in some embodiments, are pharmaceutical compositions comprising a recombinant antibody or antigen binding fragment thereof that comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

In some embodiments, the recombinant antibody or antigen binding fragment thereof further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the recombinant antibody or antigen binding fragment thereof as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservatives, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Isolated Recombinant Nucleic Acid Molecule

Disclosed herein, in some embodiments, are isolated recombinant nucleic acid molecules encoding a polypeptide of the recombinant antibody or antigen binding fragment thereof of any embodiment disclosed herein.

Disclosed herein, in some embodiments, are isolated recombinant nucleic acid molecules encoding a recombinant antibody or antigen binding fragment thereof that comprises a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2 (GTK), and wherein CDR3-L comprises an amino acid sequence of X₁-X₂-W-X₃-X₄-X₅-X₆-W-X₇-X₈ wherein X₁ is V, G, P, L, I, M, S, T, or A, X₂ is L, G, P, V, I, M, S, T, or A, X₃ is Y, F, W, V, L, I, G, or A, X₄ is S, G, T, M, N, Q, H, or A, X₅ is N, Q, S, T, D, E, H, K, R, or A, X₆ is R, S, T, Q, D, E, H, K, N, or A, X₇ is V, G, P, L, I, M, S, T, or A, and X₈ is F, Y, W, V, L, I, G, or A, wherein CDR1-H comprises the amino acid sequence of SEQ ID NO: 15, wherein CDR2-H comprises the amino acid sequence of SEQ ID NO: 16, wherein CDR3-H comprises the amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁, wherein X₉ is V, G, P, L, I, M, S, T, or A, X₁₀ is R, S, T, Q, D, E, H, K, N, or A, X₁₁ is H, R, K, G, T, S, N, Q, or A, X₁₂ is G, P, V, L, I, M, S, T, or A, X₁₃ is F, Y, W, V, L, I, G, or A, X₁₄ is G, P, V, L, I, M, S, T, or A, X₁₅ is N, Q, S, T, D, E, H, K, R, or A, X₁₆ is S, G, T, M, N, Q, H, or A, X₁₇ is Y, F, W, V, L, I, G, or A, X₁₈ is I, G, P, V, L, M, S, T, or A, X₁₉ is S, G, T, M, N, Q, H, or A, X₂₀ is W, F, Y, V, L, I, G, or A, and X₂₁ is Y, F, W, V, L, I, G, or A, wherein when CDR3-L comprises the sequence of SEQ ID NO: 3 then CDR3-H does not comprise the sequence of SEQ ID NO: 17.

Disclosed herein, in some embodiments, are isolated recombinant nucleic acid molecules encoding a recombinant antibody or antigen binding fragment thereof that comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

### Methods of Treatment

Disclosed herein, in some embodiments, are methods of treating cancer in a subject in need thereof comprising administering to the subject the recombinant antibody or antigen binding fragment thereof of any embodiment disclosed herein.

Disclosed herein, in some embodiments, are methods of treating cancer in a subject in need thereof comprising administering to the subject a recombinant antibody or antigen binding fragment thereof that comprises a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1, wherein CDR2-L comprises the amino acid sequence of SEQ ID NO: 2 (GTK), and wherein CDR3-L comprises an amino acid sequence of X₁-X₂-W-X₃-X₄-X₅-X₆-W-X₇-X₈ wherein X₁ is V, G, P, L, I, M, S, T, or A, X₂ is L, G, P, V, I, M, S, T, or A, X₃ is Y, F, W, V, L, I, G, or A, X₄ is S, G, T, M, N, Q, H, or A, X₅ is N, Q, S, T, D, E, H, K, R, or A, X₆ is R, S, T, Q, D, E, H, K, N, or A, X₇ is V, G, P, L, I, M, S, T, or A, and X₈ is F, Y, W, V, L, I, G, or A, wherein CDR1-H comprises the amino acid sequence of SEQ ID NO: 15, wherein CDR2-H comprises the amino acid sequence of SEQ ID NO: 16, wherein CDR3-H comprises the amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁, wherein X₉ is V, G, P, L, I, M, S, T, or A, X₁₀ is R, S, T, Q, D, E, H, K, N, or A, X₁₁ is H, R, K, G, T, S, N, Q, or A, X₁₂ is G, P, V, L, I, M, S, T, or A, X₁₃ is F, Y, W, V, L, I, G, or A, X₁₄ is G, P, V, L, I, M, S, T, or A, X₁₅ is N, Q, S, T, D, E, H, K, R, or A, X₁₆ is S, G, T, M, N, Q, H, or A, X₁₇ is Y, F, W, V, L, I, G, or A, X₁₈ is I, G, P, V, L, M, S, T, or A, X₁₉ is S, G, T, M, N, Q, H, or A, X₂₀ is W, F, Y, V, L, I, G, or A, and X₂₁ is Y, F, W, V, L, I, G, or A, wherein when CDR3-L comprises the sequence of SEQ ID NO: 3 then CDR3-H does not comprise the sequence of SEQ ID NO: 17.

Disclosed herein, in some embodiments, are methods of treating cancer in a subject in need thereof comprising administering to the subject a recombinant antibody or antigen binding fragment thereof that comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic or gastric. In some embodiments, the cancer has cells that express EGFR. In some embodiments, the cancer has cells that express PSMA.

In some embodiments, are methods of treating triple-negative breast cancer (TNBC), urothelial cancer (UC), squamous cell carcinoma of the head and neck (SCCHN), non-small cell lung cancer (NSCLC), small cell lung cancer (SCLC), gastric cancer, esophageal cancer, head and neck cancer, prostate cancer, or endometrial cancer in a subject need in need thereof comprising administering to the subject a recombinant antibody or antigen binding fragment thereof of any embodiment disclosed herein. In some embodiments, are methods of treating breast cancer, lung cancer, urothelial cancer, endometrial cancer, ovarian cancer, prostate cancer, pancreatic cancer, gastric cancer, colon cancer, head and neck cancer, liver cancer, cervical cancer, bladder cancer, kidney cancer, pancreatic cancer, glioblastoma, and glioma in a subject need in need thereof comprising administering to the subject a recombinant antibody or antigen binding fragment thereof of any embodiment as disclosed herein.

### Production of Antibodies

In some embodiments, the recombinant antibodies or antigen binding fragments thereof as described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F™, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a recombinant antibody or antigen binding fragment thereof of any embodiment disclosed herein.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EMBODIMENTS

Embodiment 1 comprises a recombinant antibody or antigen binding fragment thereof that comprises a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 1; wherein CDR2-L comprises the amino acid sequence of SEQ ID NO: 2 (GTK); wherein CDR3-L comprises an amino acid sequence of X₁-X₂-W-X₃-X₄-X₅-X₆-W-X₇-X₈; wherein X₁ is V, G, P, L, I, M, S, T, or A; X₂ is L, G, P, V, I, M, S, T, or A; X₃ is Y, F, W, V, L, I, G, or A; X₄ is S, G, T, M, N, Q, H, or A; X₅ is N, Q, S, T, D, E, H, K, R, or A; X₆ is R, S, T, Q, D, E, H, K, N, or A; X₇ is V, G, P, L, I, M, S, T, or A; and X₈ is F, Y, W, V, L, I, G, or A; wherein CDR1-H comprises the amino acid sequence of SEQ ID NO: 15; wherein CDR2-H comprises the amino acid sequence of SEQ ID NO: 16; wherein CDR3-H comprises the amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁; wherein X₉ is V, G, P, L, I, M, S, T, or A; X₁₀ is R, S, T, Q, D, E, H, K, N, or A; X₁₁ is H, R, K, G, T, S, N, Q, or A; X₁₂ is G, P, V, L, I, M, S, T, or A; X₁₃ is F, Y, W, V, L, I, G, or A; X₁₄ is G, P, V, L, I, M, S, T, or A; X₁₅ is N, Q, S, T, D, E, H, K, R, or A; X₁₆ is S, G, T, M, N, Q, H, or A; X₁₇ is Y, F, W, V, L, I, G, or A; X₁₈ is I, G, P, V, L, M, S, T, or A; X₁₉ is S, G, T, M, N, Q, H, or A; X₂₀ is W, F, Y, V, L, I, G, or A; and X₂₁ is Y, F, W, V, L, I, G, or A; and wherein when CDR3-L comprises the amino acid sequence of SEQ ID NO: 3 then CDR3-H does not comprise the amino acid sequence of SEQ ID NO: 17.

Embodiment 2 comprises the recombinant antibody or antigen binding fragment thereof of embodiment 1, wherein X₁ is V, G, L, I, or A; X₂ is L, V, I, or A; X₃ is Y, W, F, or A; X₄ is S, G, T, or A; X₅ is N, Q, D, E, or A; X₆ is R, K, or A; X₇ is V, G, L, I, or A; X₈ is F, Y, W, or A; X₉ is V, G, L, I, or A; X₁₀ is R, K, or A; X₁₂ is G, S, T, or A; X₁₃ is F, Y, W, or A; X₁₄ is G, S, T, or A; X₁₅ is N, Q, D, E, or A; X₁₆ is S, G, T, or A; X₁₇ is Y, W, F, or A; X₁₈ is I, V, L, or A; X₁₉ is S, G, T, or A; X₂₀ is W, Y, F, or A; and X₂₁ is Y, W, F, or A.

Embodiment 3 comprises the recombinant antibody or antigen binding fragment thereof of embodiments 1 or 2, wherein X₈ is F.

Embodiment 4 comprises the recombinant antibody or antigen binding fragment thereof of any one of embodiments 1 to 3, wherein X₁₀ is R; X₁₁ is H; X₁₃ is F; X₁₈ is I; X₁₉ is S; and X₂₀ is W.

Embodiment 5 comprises the recombinant antibody or antigen binding fragment thereof of embodiment 1, wherein CDR3-L comprises an amino acid sequence selected from SEQ ID NOs: 3, 5-6, 8-11, and 13-14.

Embodiment 6 comprises the recombinant antibody or antigen binding fragment thereof of embodiment 1, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17.

Embodiment 7 comprises the recombinant antibody or antigen binding fragment thereof of embodiment 1, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; and CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17.

Embodiment 8 comprises the recombinant antibody or antigen binding fragment thereof of embodiment 1, wherein CDR3-H comprises an amino acid sequence selected from SEQ ID NOs: 17, 19-22, 24-30, and 32-33.

Embodiment 9 comprises the recombinant antibody or antigen binding fragment thereof of embodiment 1, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

Embodiment 10 comprises the recombinant antibody or antigen binding fragment thereof of embodiment 1, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

Embodiment 11 comprises a recombinant antibody or antigen binding fragment thereof that comprises a CD3 binding domain, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of: CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 7, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33; and CDR1-L: SEQ ID NO: 4, CDR2-L: SEQ ID NO: 2 (GTK), CDR3-L: SEQ ID NO: 5, and CDR1-H: SEQ ID NO: 18, CDR2-H: SEQ ID NO: 16, CDR3-H: SEQ ID NO: 178.

Embodiment 12 comprises the recombinant antibody or antigen binding fragment thereof of any one of the preceding embodiments, wherein the immunoglobulin light chain of the CD3 binding domain comprises a variable domain of an immunoglobulin kappa (IgK) or immunoglobulin lambda (IgL) light chain.

Embodiment 13 comprises the recombinant antibody or antigen binding fragment thereof of any one of the preceding embodiments, wherein the immunoglobulin heavy chain of the CD3 binding domain comprises a variable domain of an IgG1, IgG2, IgG3, or IgG4 heavy chain.

Embodiment 14 comprises the recombinant antibody or antigen binding fragment thereof of any one of the preceding embodiments, wherein the CD3 binding domain comprises a Fab, Fab′, (Fab′)₂, or a single chain variable fragment (scFv).

Embodiment 15 comprises the recombinant antibody or antigen binding fragment thereof of embodiment 14, wherein the CD3 binding domain is the scFv.

Embodiment 16 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 37.

Embodiment 17 comprises the recombinant antibody or antigen binding fragment thereof any one of embodiments 1 to 15, wherein the CD3 binding domain comprises an amino acid sequence with at least 90%, 95%, 96%, 97%, 98%, 99%, or 100% identity to SEQ ID NO: 38.

Embodiment 18 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 39.

Embodiment 19 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 40.

Embodiment 20 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 41.

Embodiment 21 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 42.

Embodiment 22 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 43.

Embodiment 23 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 44.

Embodiment 24 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 45.

Embodiment 25 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 46.

Embodiment 26 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 47.

Embodiment 27 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 48.

Embodiment 28 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 49.

Embodiment 29 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 50.

Embodiment 30 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 51.

Embodiment 31 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 52.

Embodiment 32 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 53.

Embodiment 33 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 54.

Embodiment 34 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 55.

Embodiment 35 comprises the recombinant antibody or antigen binding fragment of thereof of any one of embodiments 1 to 15, wherein the CD3 binding domain comprises an amino acid sequence with at least 90%, 95%, 96%, 97%, 98%, 99%, or 100% identity to SEQ ID NO: 56.

Embodiment 36 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 57.

Embodiment 37 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 58.

Embodiment 38 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 59.

Embodiment 39 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 60.

Embodiment 40 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 61.

Embodiment 41 comprises the recombinant antibody or antigen binding fragment thereof of any one of embodiments 1 to 15, wherein the CD3 binding domain comprises an amino acid sequence with at least 90%, 95%, 96%, 97%, 98%, 99%, or 100% identity to SEQ ID NO: 63.

Embodiment 42 comprises the recombinant antibody or antigen binding fragment thereof of any one of the preceding embodiments, wherein the recombinant antibody or antigen binding fragment thereof further comprises an antigen binding domain.

Embodiment 43 comprises the recombinant antibody or antigen binding fragment thereof of embodiment 42, wherein the antigen binding domain is selected from a tumor-associated calcium signal transducer 2 (TROP2) binding domain, an epidermal growth factor receptor (EGFR) binding domain, and a prostate-specific membrane antigen (PSMA) binding domain.

Embodiment 44 comprises the recombinant antibody or antigen binding fragment thereof of embodiments 42 or 43, wherein the antigen binding domain is a TROP2 binding domain.

Embodiment 45 comprises the recombinant antibody or antigen binding fragment thereof of embodiment 44, wherein the TROP2 binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein: CDR1-L comprises the amino acid sequence of SEQ ID NO: 64; CDR2-L comprises the amino acid sequence of SEQ ID NO: 65 (SAS); CDR3-L comprises the amino acid sequence of SEQ ID NO: 66; CDR1-H comprises the amino acid sequence of SEQ ID NO: 67; CDR2-H comprises the amino acid sequence of SEQ ID NO: 68; and CDR3-H comprises an amino acid sequence selected from any one of SEQ ID NOs: 69-71.

Embodiment 46 comprises the recombinant antibody or antigen binding fragment thereof of embodiments 44 or 45, wherein the TROP2 binding domain comprises a Fab, Fab′, (Fab′)₂, or a single chain variable fragment (scFv).

Embodiment 47 comprises the recombinant antibody or antigen binding fragment thereof of any one of embodiments 43 to 46, wherein the TROP2 binding domain is a Fab.

Embodiment 48 comprises the recombinant antibody or antigen binding fragment thereof of any one of embodiments 45 to 47, wherein the immunoglobulin light chain of the TROP2 binding domain comprises a variable domain of an immunoglobulin kappa (IgK) or immunoglobulin lambda (IgL) light chain.

Embodiment 49 comprises the recombinant antibody or antigen binding fragment thereof of any one of embodiments 45 to 48, wherein the immunoglobulin heavy chain of the TROP2 binding domain comprises a variable domain of an IgG1, IgG2, IgG3, or IgG4 heavy chain.

Embodiment 50 comprises the recombinant antibody or antigen binding fragment thereof of any one of embodiments 45 to 49, wherein the N-term of the immunoglobulin heavy chain of the TROP2 binding domain is bound to the C-term of the immunoglobulin light chain of the CD3 binding domain.

Embodiment 51 comprises the recombinant antibody or antigen binding fragment thereof of any one of embodiments 45 to 49, wherein the C-term of the immunoglobulin heavy chain of the TROP2 binding domain is bound to the N-term of the immunoglobulin light chain of the CD3 binding domain.

Embodiment 52 comprises the recombinant antibody or antigen binding fragment thereof of any one of embodiments 45 to 49, wherein the N-term of the immunoglobulin heavy chain of the TROP2 binding domain is bound to the C-term of the immunoglobulin heavy chain of the CD3 binding domain.

Embodiment 53 comprises the recombinant antibody or antigen binding fragment thereof of any one of embodiments 45 to 49, wherein the C-term of the immunoglobulin heavy chain of the TROP2 binding domain is bound to the N-term of the immunoglobulin heavy chain of the CD3 binding domain.

Embodiment 54 comprises the recombinant antibody or antigen binding fragment thereof of any one of embodiments 45 to 49, wherein the N-term of the immunoglobulin light chain of the TROP2 binding domain is bound to the C-term of the immunoglobulin light chain of the CD3 binding domain.

Embodiment 55 comprises the recombinant antibody or antigen binding fragment thereof of any one of embodiments 45 to 49, wherein the C-term of the immunoglobulin light chain of the TROP2 binding domain is bound to the N-term of the immunoglobulin light chain of the CD3 binding domain.

Embodiment 56 comprises the recombinant antibody or antigen binding fragment thereof of any one of embodiments 45 to 49, wherein the N-term of the immunoglobulin light chain of the TROP2 binding domain is bound to the C-term of the immunoglobulin heavy chain of the CD3 binding domain.

Embodiment 57 comprises the recombinant antibody or antigen binding fragment thereof of any one of embodiments 45 to 49, wherein the C-term of the immunoglobulin light chain of the TROP2 binding domain is bound to the N-term of the immunoglobulin heavy chain of the CD3 binding domain.

Embodiment 58 comprises the recombinant antibody or antigen binding fragment thereof of any one of embodiments 43 to 57, wherein the CD3 binding domain is a scFv and the TROP2 binding domain is a Fab or Fab′.

Embodiment 59 comprises the recombinant antibody or antigen binding fragment thereof of embodiment 58, wherein the scFv is bound to the immunoglobulin heavy chain of the Fab or Fab′.

Embodiment 60 comprises the recombinant antibody or antigen binding fragment thereof of embodiment 58, wherein the scFv is bound to the immunoglobulin light chain of the Fab or Fab′.

Embodiment 61 comprises the recombinant antibody or antigen binding fragment thereof of embodiment 58, wherein the immunoglobulin light chain of the scFv is bound to the immunoglobulin heavy chain of the Fab or Fab′.

Embodiment 62 comprises the recombinant antibody or antigen binding fragment thereof of embodiment 58, wherein the immunoglobulin light chain of the scFv is bound to the immunoglobulin light chain of the Fab or Fab′.

Embodiment 63 comprises the recombinant antibody or antigen binding fragment thereof of embodiment 58, wherein the immunoglobulin heavy chain of the scFv is bound to the immunoglobulin heavy chain of the Fab or Fab′.

Embodiment 64 comprises the recombinant antibody or antigen binding fragment thereof of embodiment 58, wherein the immunoglobulin heavy chain of the scFv is bound to the immunoglobulin light chain of the Fab or Fab′.

Embodiment 65 comprises the recombinant antibody or antigen binding fragment thereof of any one of embodiments 45 to 64, wherein the immunoglobulin light chain of the TROP2 binding domain comprises an amino acid sequence with at least 90%, 95%, 96%, 97%, 98%, 99%, or 100% identity to SEQ ID NO: 72.

Embodiment 66 comprises the recombinant antibody or antigen binding fragment thereof of any one of embodiments 45 to 65, wherein the immunoglobulin heavy chain of the TROP2 binding domain comprises an amino acid sequence with at least 90%, 95%, 96%, 97%, 98%, 99%, or 100% identity to any one of SEQ ID NOs: 73-75.

Embodiment 67 comprises the recombinant antibody or antigen binding fragment thereof of any one of embodiments 45 to 66, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 73.

Embodiment 68 comprises the recombinant antibody or antigen binding fragment thereof of any one of embodiments 45 to 66, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 74.

Embodiment 69 comprises the recombinant antibody or antigen binding fragment thereof of any one of embodiments 45 to 66, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 75.

Embodiment 70 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 77.

Embodiment 71 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 78.

Embodiment 72 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 79.

Embodiment 73 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 80.

Embodiment 74 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 81.

Embodiment 75 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 82.

Embodiment 76 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 83.

Embodiment 77 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 84.

Embodiments 78 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 85.

Embodiments 79 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 86.

Embodiments 80 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 87.

Embodiments 81 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 88.

Embodiments 82 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 89.

Embodiment 83 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 90.

Embodiment 84 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 91.

Embodiment 85 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 92.

Embodiments 86 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 93.

Embodiment 87 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 94.

Embodiment 88 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 95.

Embodiment 89 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 96.

Embodiment 90 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 97.

Embodiment 91 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 98.

Embodiment 92 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 99.

Embodiment 93 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 100.

Embodiment 94 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 101.

Embodiment 95 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 105.

Embodiment 96 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 106.

Embodiment 97 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 107.

Embodiment 98 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 111.

Embodiment 99 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 112.

Embodiment 100 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 113.

Embodiment 101 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 109.

Embodiment 102 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 176.

Embodiment 103 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 177.

Embodiment 104 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 77.

Embodiment 105 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 78.

Embodiment 106 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 79.

Embodiment 107 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 80.

Embodiment 108 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 83.

Embodiment 109 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 84.

Embodiment 110 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 85.

Embodiment 111 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 86.

Embodiment 112 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 87.

Embodiment 113 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 88.

Embodiment 114 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 91.

Embodiment 115 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 92.

Embodiment 116 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 93.

Embodiment 117 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 95.

Embodiment 118 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 96.

Embodiment 119 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 97.

Embodiment 120 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 98.

Embodiment 121 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 100.

Embodiment 122 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 101.

Embodiment 123 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 105.

Embodiment 124 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 109.

Embodiment 125 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 176.

Embodiment 126 comprises the recombinant antibody or antigen binding fragment thereof of embodiments 1 or 11, wherein the recombinant antibody or antigen binding fragment thereof comprises the amino acid sequences of SEQ ID NO: 72 and SEQ ID NO: 177.

Embodiment 127 comprises the recombinant antibody or antigen binding fragment thereof of embodiments 42 or 43, wherein the antigen binding domain is an EGFR binding domain.

Embodiment 128 comprises the recombinant antibody or antigen binding fragment thereof of embodiment 127, wherein the EGFR binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein: CDR1-L comprises the amino acid sequence of SEQ ID NO: 115; CDR2-L comprises the amino acid sequence of SEQ ID NO: 116 (YAS); CDR3-L comprises the amino acid sequence of SEQ ID NO: 117; CDR1-H comprises the amino acid sequence of SEQ ID NO: 118; CDR2-H comprises the amino acid sequence of SEQ ID NO: 119; and CDR3-H comprises the amino acid sequence of SEQ ID NO: 120.

Embodiment 129 comprises the recombinant antibody or antigen binding fragment thereof of embodiment 128, wherein the immunoglobulin light chain of the EGFR binding domain comprises an amino acid sequence with at least 90%, 95%, 96%, 97%, 98%, 99%, or 100% identity to SEQ ID NO: 121, and the immunoglobulin heavy chain of the EGFR binding domain comprises an amino acid sequence with at least 90%, 95%, 96%, 97%, 98%, 99%, or 100% identity to SEQ ID NO: 122.

Embodiment 130 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 125 and SEQ ID NO: 126.

Embodiment 131 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 127 and SEQ ID NO: 128.

Embodiment 132 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 129 and SEQ ID NO: 130.

Embodiment 133 comprises the recombinant antibody or antigen binding fragment thereof of embodiments 1 or 11, wherein the recombinant antibody or antigen binding fragment thereof comprises the amino acid sequences of SEQ ID NO: 125 and SEQ ID NO: 126.

Embodiment 134 comprises the recombinant antibody or antigen binding fragment thereof of embodiments 1 or 11, wherein the recombinant antibody or antigen binding fragment thereof comprises the amino acid sequences of SEQ ID NO: 127 and SEQ ID NO: 128.

Embodiment 135 comprises the recombinant antibody or antigen binding fragment thereof of embodiments 42 or 43, wherein the antigen binding domain is a PSMA binding domain.

Embodiment 136 comprises the recombinant antibody or antigen binding fragment thereof of embodiment 135, wherein the PSMA binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein: CDR1-L comprises the amino acid sequence of SEQ ID NO: 131; CDR2-L comprises the amino acid sequence of SEQ ID NO: 132 (EAS); CDR3-L comprises the amino acid sequence of SEQ ID NO: 133; CDR1-H comprises the amino acid sequence of SEQ ID NO: 134; CDR2-H comprises the amino acid sequence of SEQ ID NO: 135; and CDR3-H comprises the amino acid sequence of SEQ ID NO: 136.

Embodiment 137 comprises the recombinant antibody or antigen binding fragment thereof of embodiment 136, wherein the immunoglobulin light chain of the PSMA binding domain comprises an amino acid sequence with at least 90%, 95%, 96%, 97%, 98%, 99%, or 100% identity to SEQ ID NO: 137, and the immunoglobulin heavy chain of the PSMA binding domain comprises an amino acid sequence with at least 90%, 95%, 96%, 97%, 98%, 99%, or 100% identity to SEQ ID NO: 138.

Embodiment 138 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 141 and SEQ ID NO: 142.

Embodiment 139 comprises the recombinant antibody or antigen binding fragment thereof of any one of embodiments 1 to 15, wherein the recombinant antibody or antigen binding fragment thereof comprises amino acid sequences with at least 90%, 95%, 96%, 97%, 98%, 99%, or 100% identity to SEQ ID NO: 143 and SEQ ID NO: 144.

Embodiment 140 comprises the recombinant antibody or antigen binding fragment thereof of any one of embodiments 1 to 15, the recombinant antibody or antigen binding fragment thereof comprises amino acid sequences with at least 90%, 95%, 96%, 97%, 98%, 99%, or 100% identity to SEQ ID NO: 145 and SEQ ID NO: 146.

Embodiment 141 comprises the recombinant antibody or antigen binding fragment thereof of embodiments 1 or 11, wherein the recombinant antibody or antigen binding fragment thereof comprises the amino acid sequences of SEQ ID NO: 141 and SEQ ID NO: 142.

Embodiment 142 comprises the recombinant antibody or antigen binding fragment thereof of embodiments 1 or 11, wherein the recombinant antibody or antigen binding fragment thereof comprises the amino acid sequences of SEQ ID NO: 143 and SEQ ID NO: 144.

Embodiment 143 comprises the recombinant antibody or antigen binding fragment thereof of embodiments 42 or 43, wherein the CD3 binding domain and the antigen binding domain are connected by a linker.

Embodiment 144 comprises the recombinant antibody or antigen binding fragment thereof of embodiment 143, wherein the linker is at least 5 amino acids in length.

Embodiment 145 comprises the recombinant antibody or antigen binding fragment thereof of embodiment 143, wherein the linker is no more than 10 amino acids in length.

Embodiment 146 comprises the recombinant antibody or antigen binding fragment thereof of embodiment 143, wherein the linker is no more than 30 amino acids in length.

Embodiment 147 comprises the recombinant antibody or antigen binding fragment thereof of embodiment 143, wherein the linker is at least 5 amino acids and no more than 30 amino acids in length.

Embodiment 148 comprises the recombinant antibody or antigen binding fragment thereof of embodiment 143, wherein the linker is 5 amino acids in length.

Embodiment 149 comprises the recombinant antibody or antigen binding fragment thereof of embodiment 143, wherein the linker comprises the amino acid sequence of SEQ ID NO: 147.

Embodiment 150 comprises the recombinant antibody or antigen binding fragment thereof of embodiment 143, wherein the linker comprises the amino acid sequence of SEQ ID NO: 148.

Embodiment 151 comprises the recombinant antibody or antigen binding fragment thereof of embodiment 143, wherein the linker comprises the amino acid sequence of SEQ ID NO: 149.

Embodiment 152 comprises the recombinant antibody or antigen binding fragment thereof of embodiment 143, wherein the linker comprises an amino acid sequence selected from any one of SEQ ID NOs: 147-175.

Embodiment 153 comprises a pharmaceutical composition comprising: (i) the recombinant antibody or antigen binding fragment thereof of any one of embodiments 1 to 152; and (ii) a pharmaceutically acceptable excipient.

Embodiment 154 comprises an isolated recombinant nucleic acid molecule encoding a polypeptide of the recombinant antibody or antigen binding fragment thereof of any one of embodiments 1 to 152.

Embodiment 155 comprises a vector comprising the isolated recombinant nucleic acid molecule of embodiment 154.

Embodiment 156 comprises a host cell comprising the isolated recombinant nucleic acid molecule of embodiment 154.

Embodiment 157 comprises a method of treating cancer in a subject in need thereof comprising administering to the subject the recombinant antibody or antigen binding fragment thereof of any one of embodiments 1 to 152.

Embodiment 158 comprises a kit comprising the pharmaceutical composition of embodiment 153.

### EXAMPLES

### Example 1: Alanine Scanning in TROP2 TCE Constructs

Alanine scanning of the light and heavy chains of anti-CD3 scFv CDR3s of the TROP2 T cell engager (TCE) constructs were carried out in order to establish CDR3 related sequence activity relationships (SAR). Alanine scanning was accomplished by mutating each individual residue in the CDR3 light chain (LC) region and the CDR3 heavy chain (HC) region of the anti-CD3 scFv starting sequence to alanine. The amino acid sequences of the non-mutated (or “wild-type”) and mutated TCE constructs are provided in Table 7. TCE-1 has the “wild-type” or non-mutated anti-CD3 CDR3 sequences. TCE-2 to TCE-16 have individual alanine mutations in the anti-CD3 CDR3 heavy chain, and TCE-17 to TCE-26 have individual alanine mutations in the anti-CD3 CDR3 light chain. TCE-30 to TCE-32 and TCE-36 to TCE-38 include alanine mutations in both the anti-CD3 CDR3 and the anti-TROP2 CDR3 heavy chains.

### Example 2: Binding of TROP2 TCEs to CD3ε and TROP2

### Equilibrium Binding to CD3ε and TROP2 by ELISA

The wild-type and alanine mutated TCEs of Example 1 were evaluated for their ability to bind CD3ε and TROP2 using a standard enzyme-linked immunosorbent assay (ELISA) format. Briefly, biotinylated CD3ε or biotinylated TROP2 was captured on neutravidin coated plates. The TCE constructs diluted in buffer were then added to the antigen coated plates. Binding was detected using a standard horse radish peroxidase secondary antibody. The concentration of the TCE required to achieve 50% maximal signal (EC₅₀) was calculated.

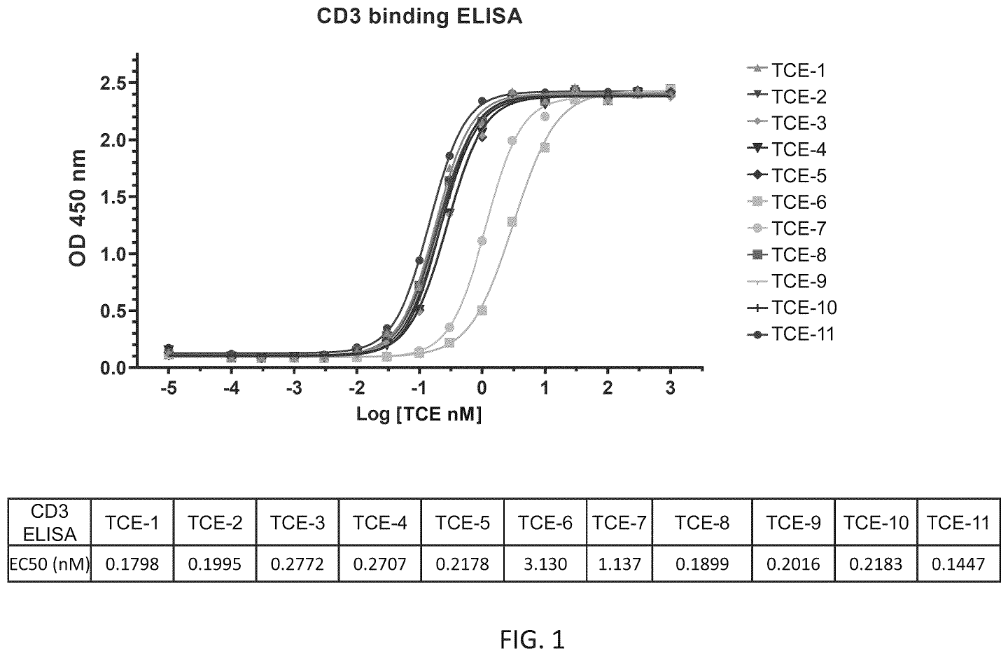

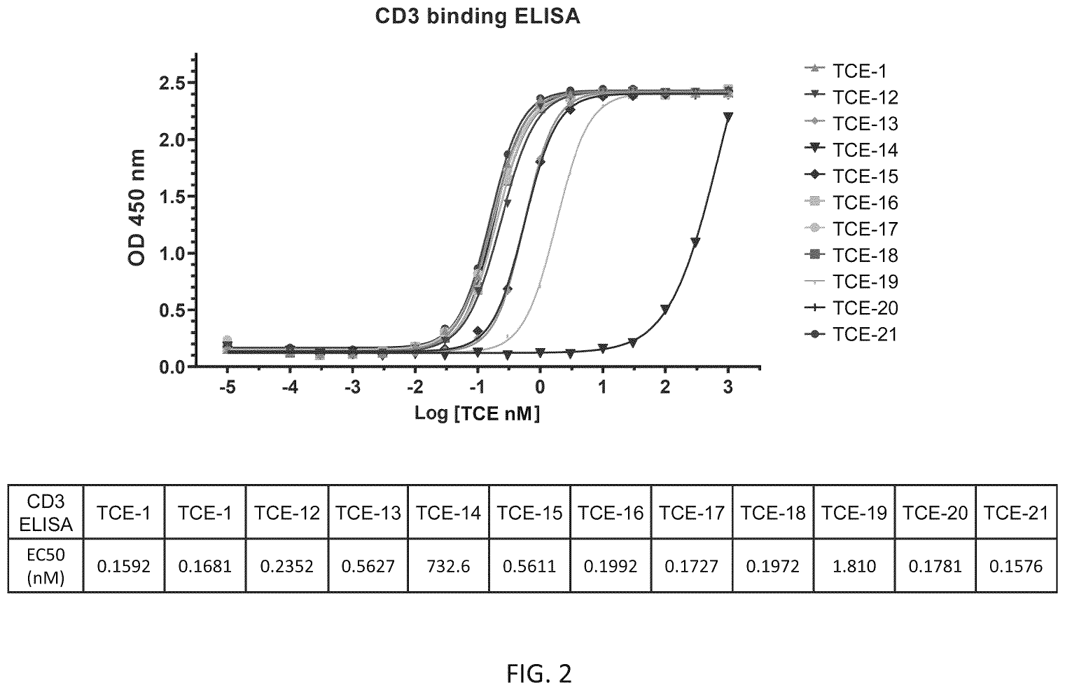

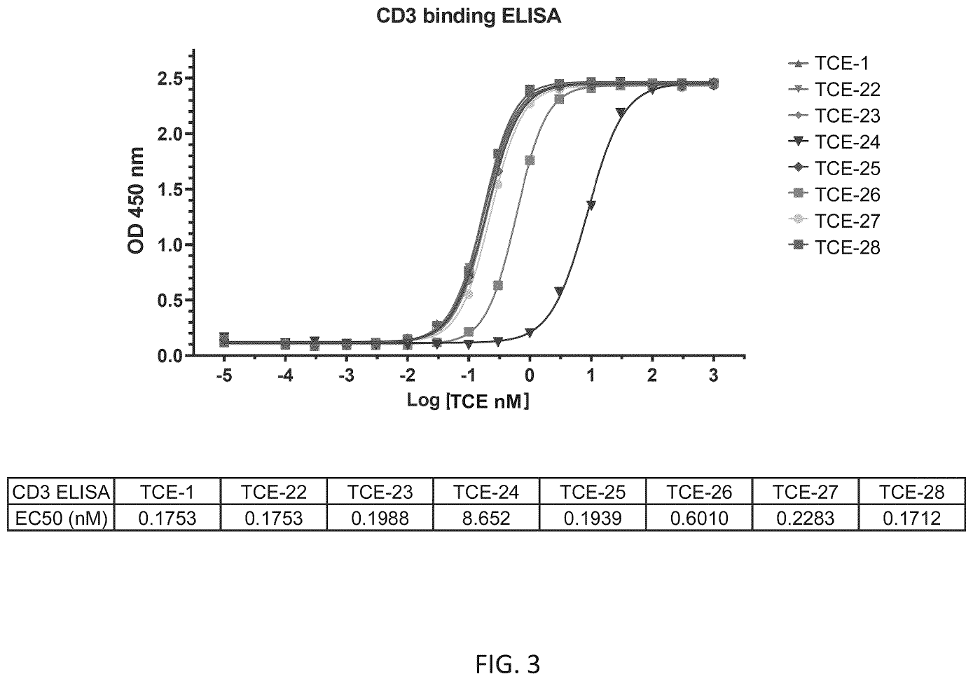

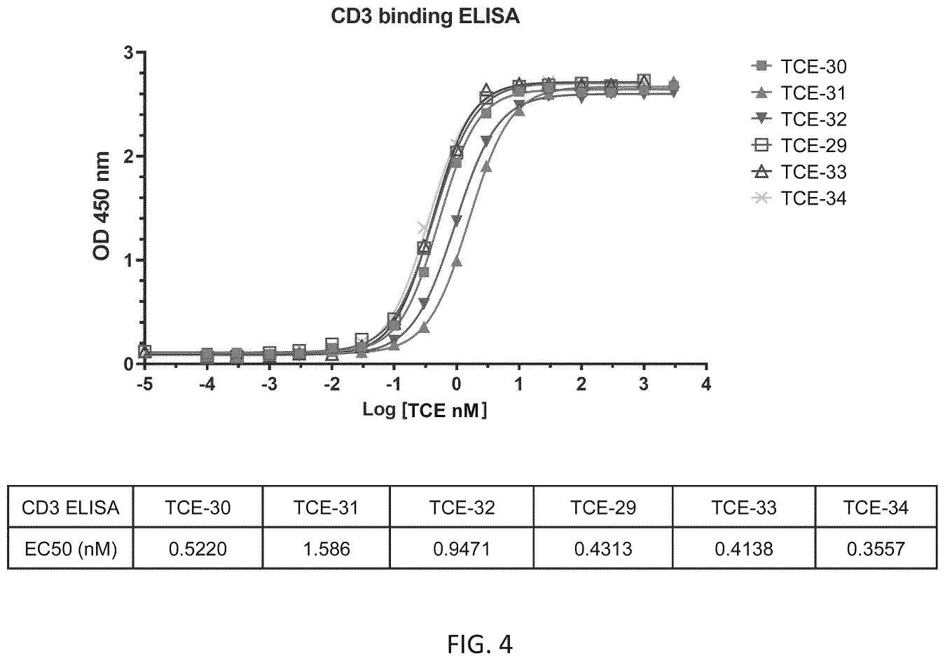

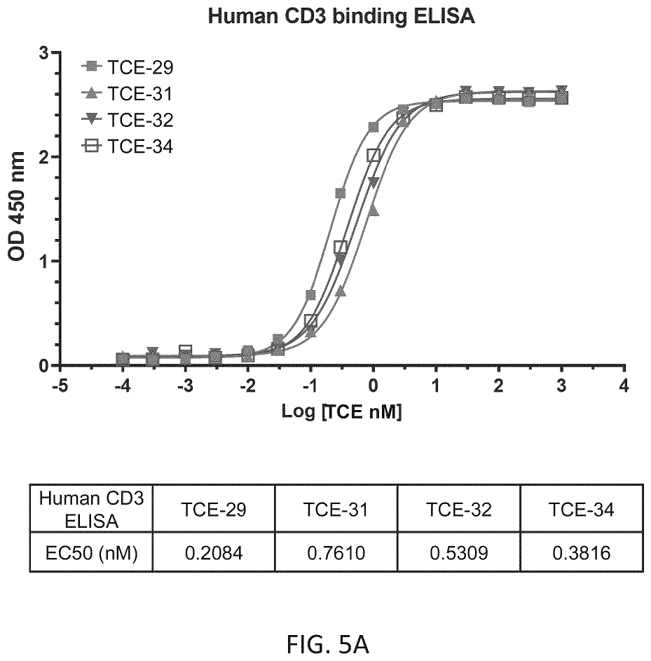

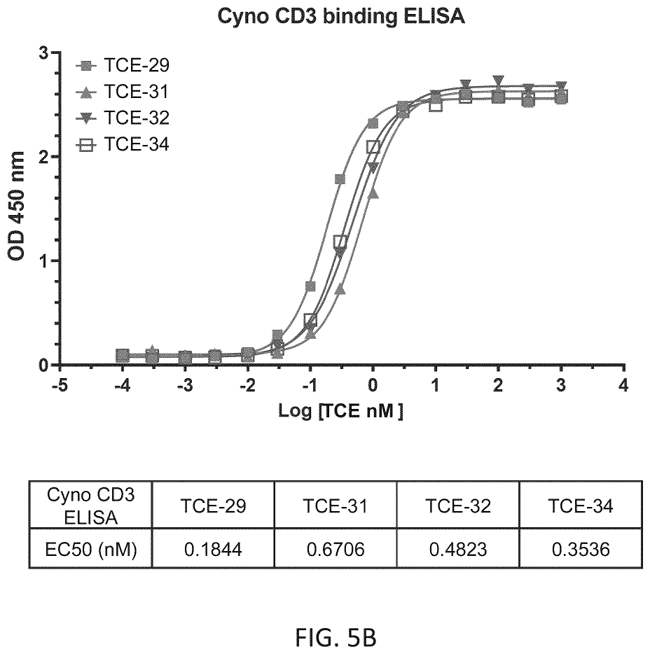

Binding curves for TCE-1 to TCE-11 binding to human CD3ε are shown in FIG. 1 along with calculated EC₅₀s. Binding curves for TCE-1 and TCE-12 to TCE-21 binding to human CD3ε are shown in FIG. 2 along with calculated EC₅₀s. Binding curves and EC₅₀s for TCE-1 and TCE-22 to TCE-28 binding to human CD3ε are shown in FIG. 3. FIG. 4 shows binding curves and EC₅₀s for binding of TCE-29 to TCE-34 binding to CD3E. FIGS. 5A-5B show binding curves and EC₅₀s for binding of TCE-29, TCE-31, TCE-32, and TCE-34 to human CD3 (FIG. 5A) and cyno CD3 (FIG. 5B) as measured by ELISA.

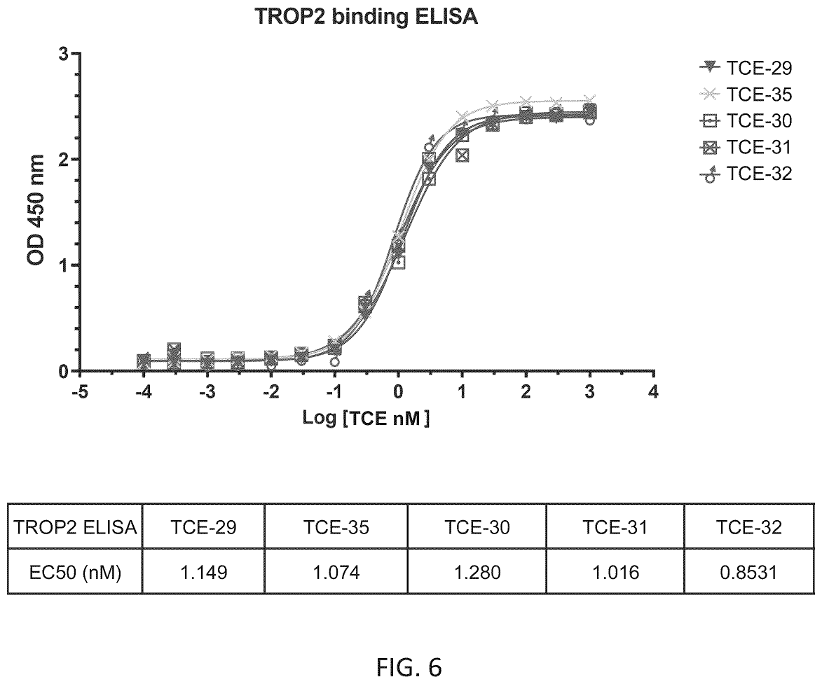

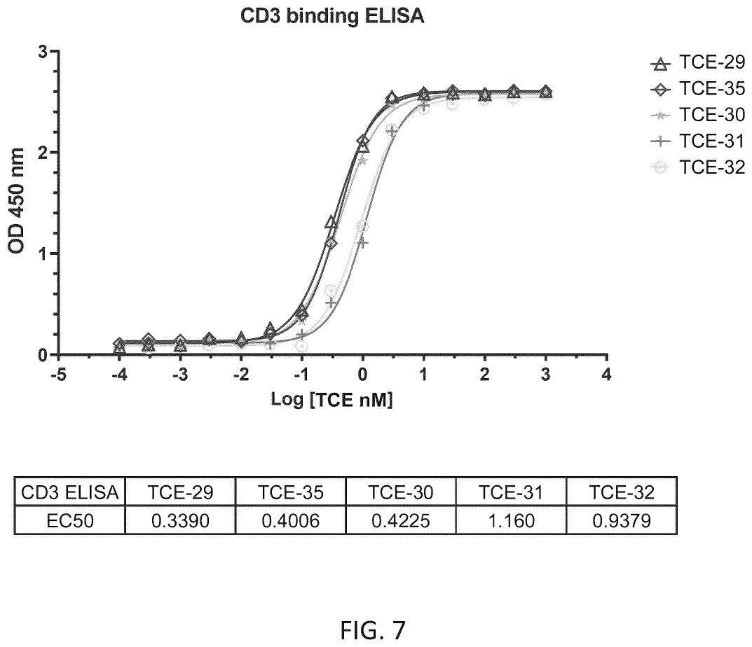

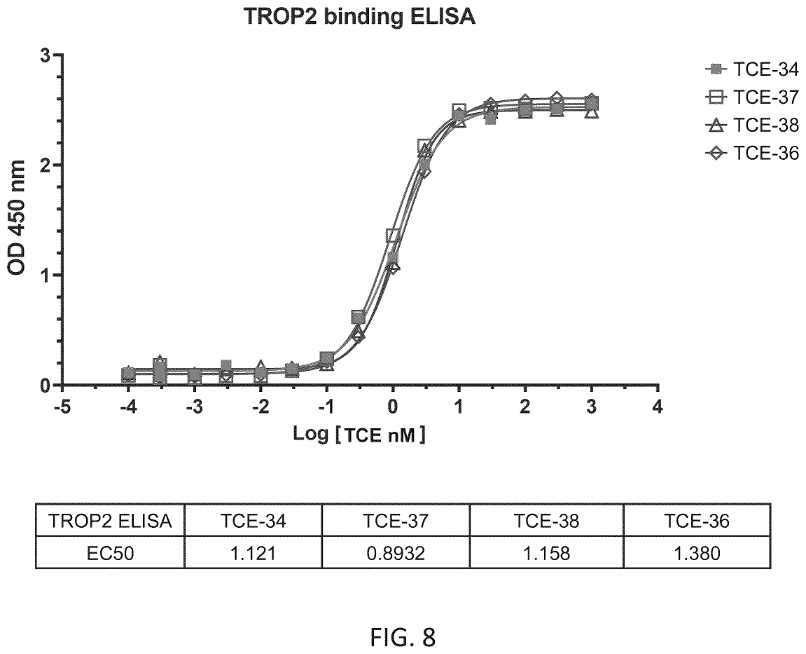

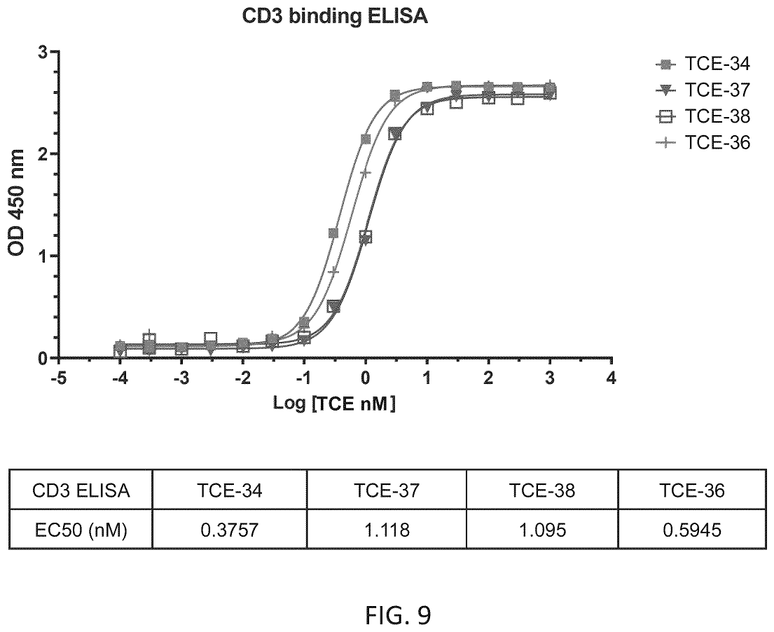

Binding curves and EC₅₀s for TROP2 binding by TCE-29, TCE-30, TCE-31, TCE-32, and TCE-35 as measured by ELISA are shown in FIG. 6. Binding curves and EC₅₀s for CD3 binding by TCE-29, TCE-30, TCE-31, TCE-32, and TCE-35 as measured by ELISA are shown in FIG. 7. Binding curves and EC₅₀s for TROP2 binding by TCE-34, TCE-37, TCE-38, and TCE-36 as measured by ELISA are shown in FIG. 8. Binding curves for CD3 binding by TCE-34, TCE-37, TCE-38, and TCE-36 as measured by ELISA are shown in FIG. 9.

### Kinetic Binding to CD3ε and TROP2 Via BLI

The wild-type (non-mutated) and alanine mutated TCEs were evaluated for their ability to bind CD3ε or TROP2 via kinetic binding measurements using biolayer interferometry (BLI). Briefly, biotinylated CD3ε or TROP2 was loaded onto a streptavidin coated Octet® SAX biosensor, quenched in biocytin, and baselined in buffer. The TCEs diluted in buffer were then associated onto the antigen loaded biosensors. Sensors were then transferred to buffer where the TCEs were then dissociated from the sensors. Association and dissociation rates were measured in real time using an Octet® instrument. Exemplary experimental conditions and steps used for the kinetic binding measurements with CD3ε are shown in Table 17.

TABLE 17

| Step | Time |
|---|---|
| Baseline: Octet buffer | 60 seconds |
| Loading: 10 nM human | 300 seconds |
| CD3ε-biotin | |
| Biocytin quench | 300 seconds |
| (100 μM) | |
| Baseline: Octet buffer | 300 seconds |
| Association: 50 nM TCE | 300 seconds |
| Dissociation: Octet buffer | 600 seconds |

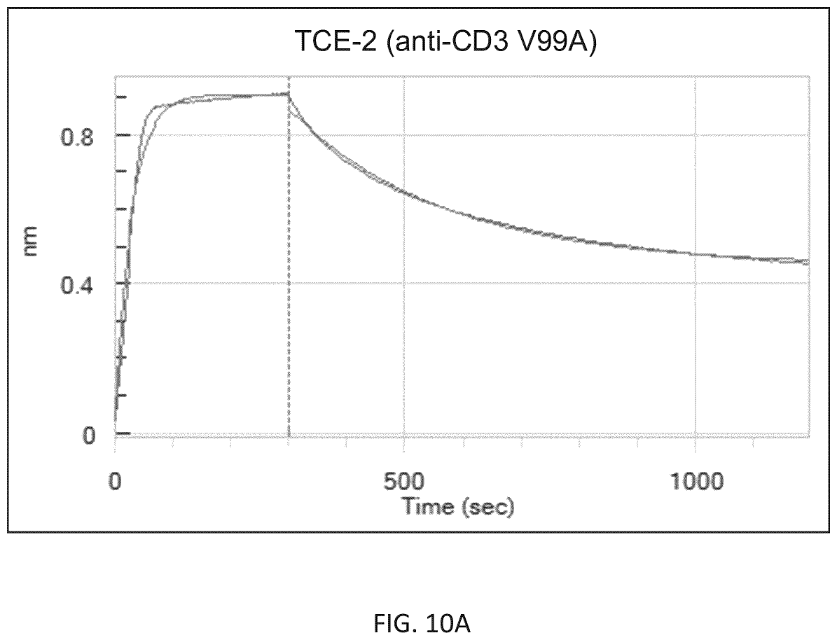

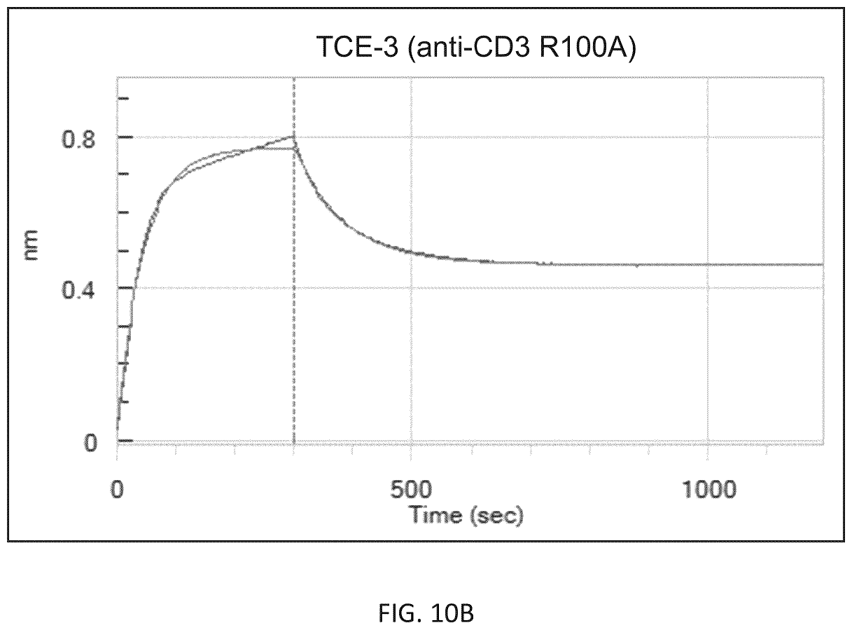

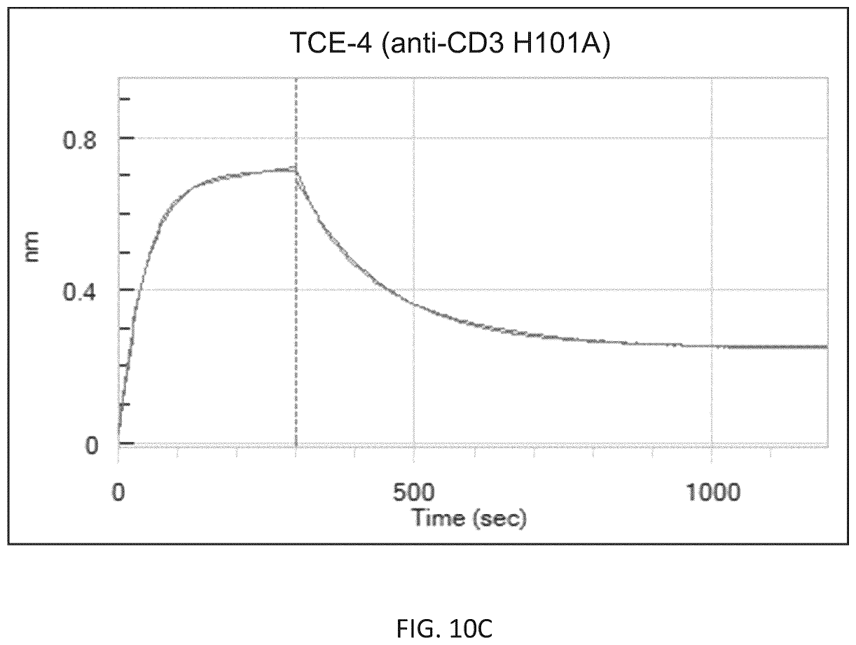

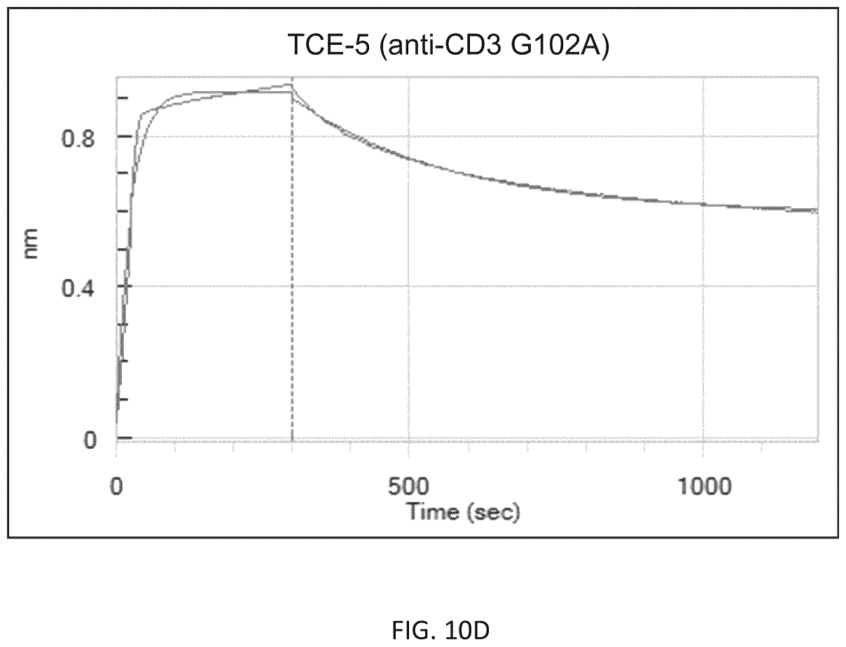

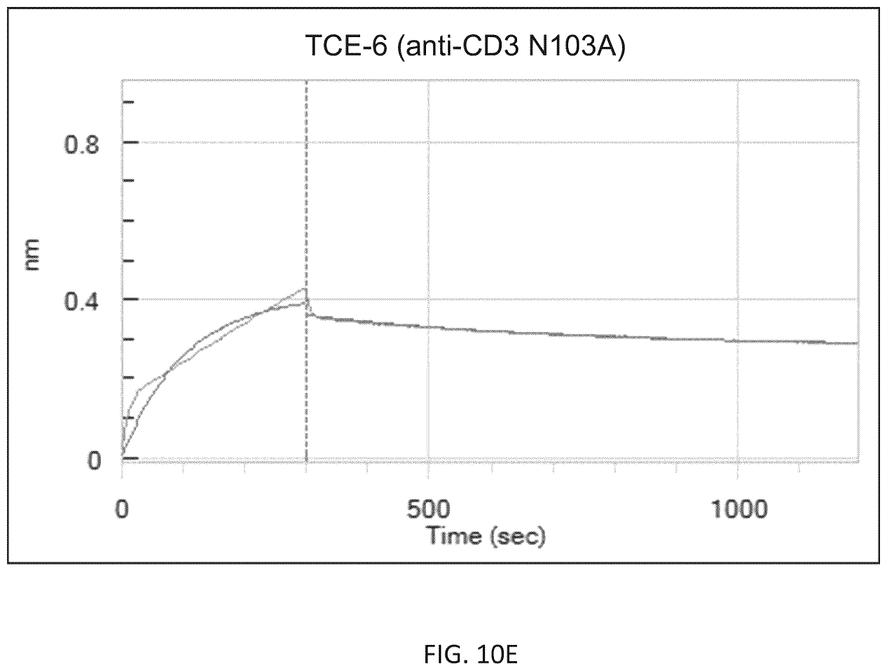

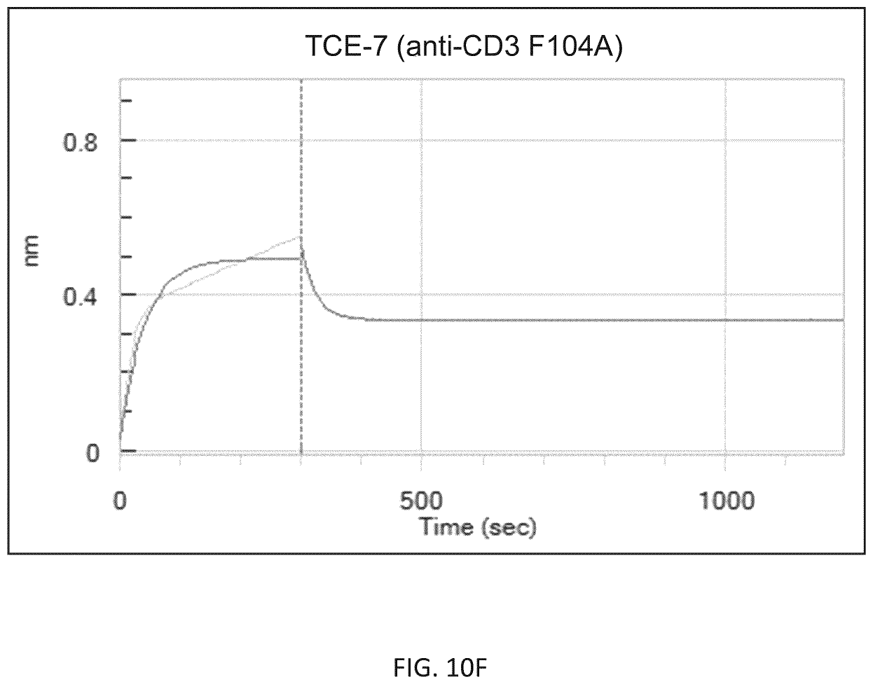

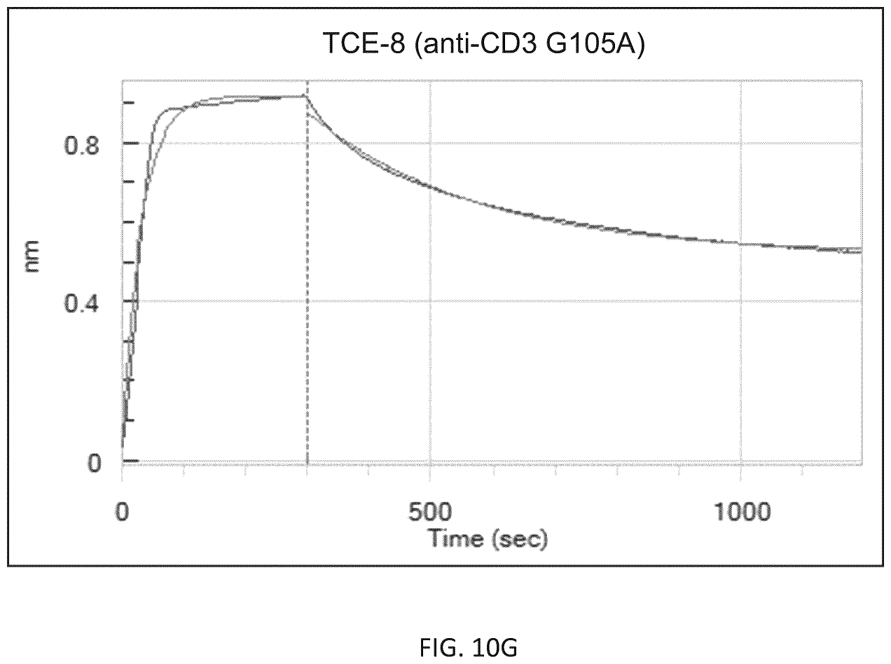

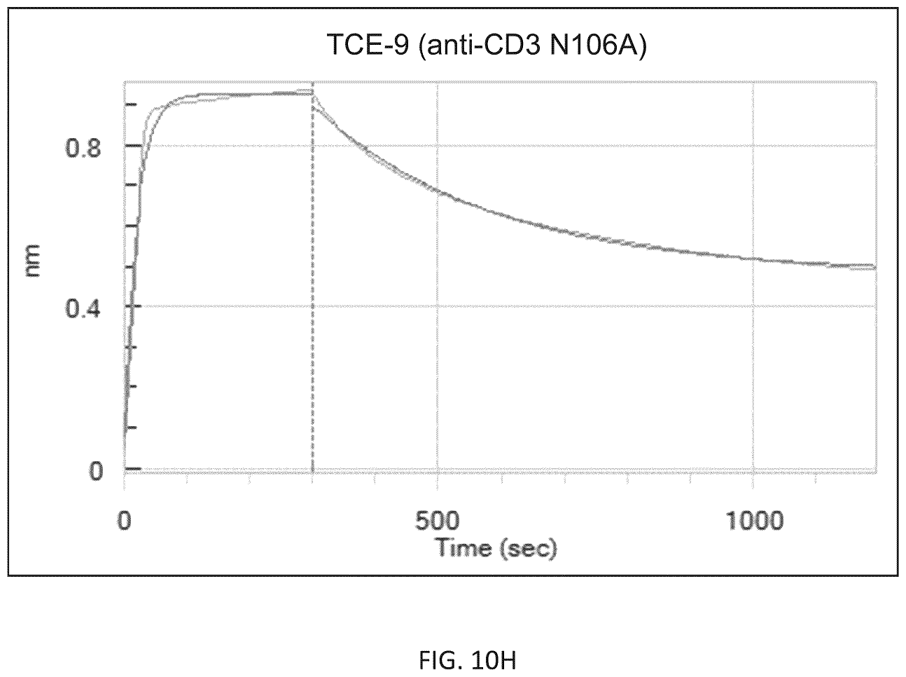

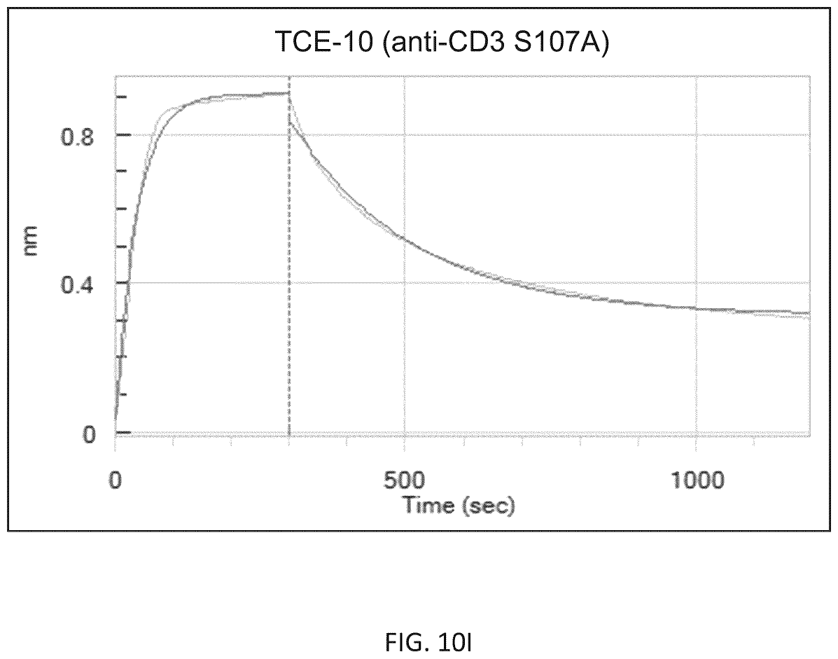

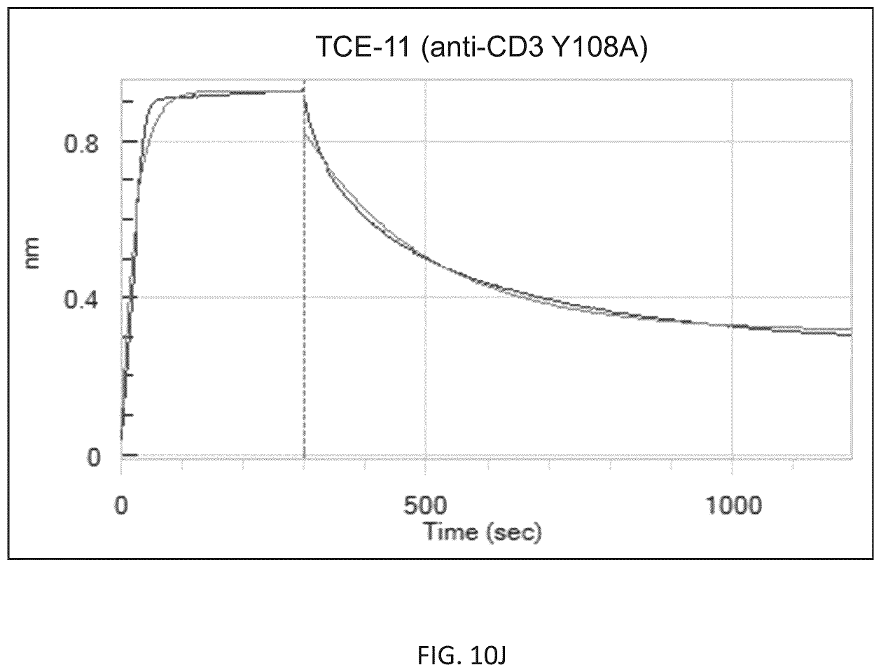

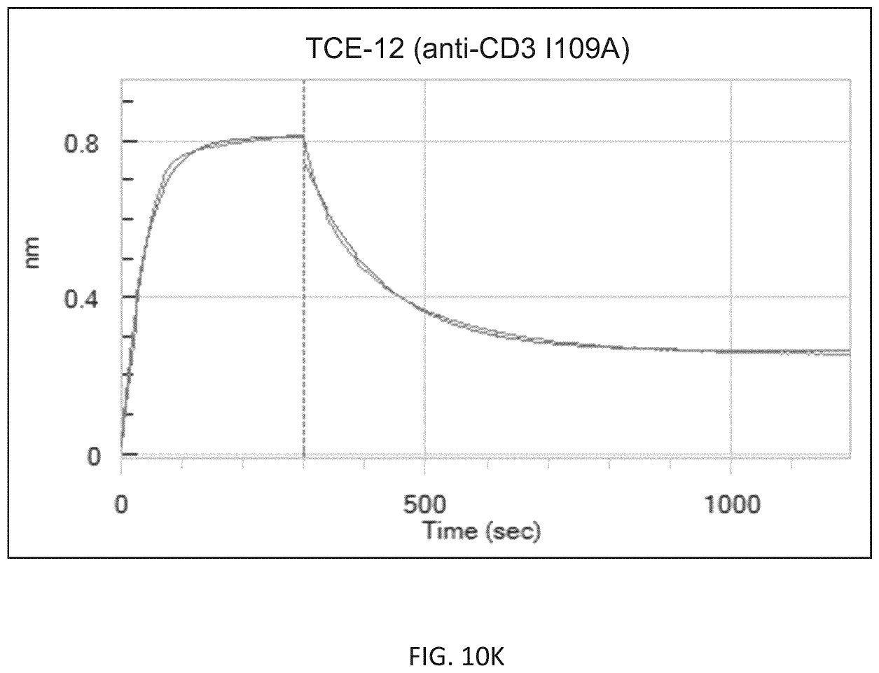

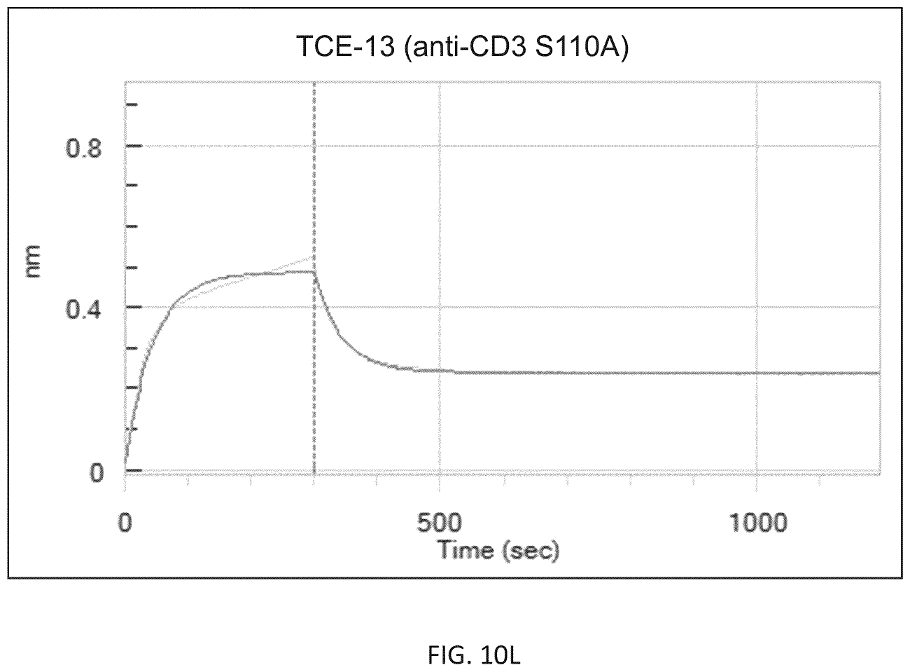

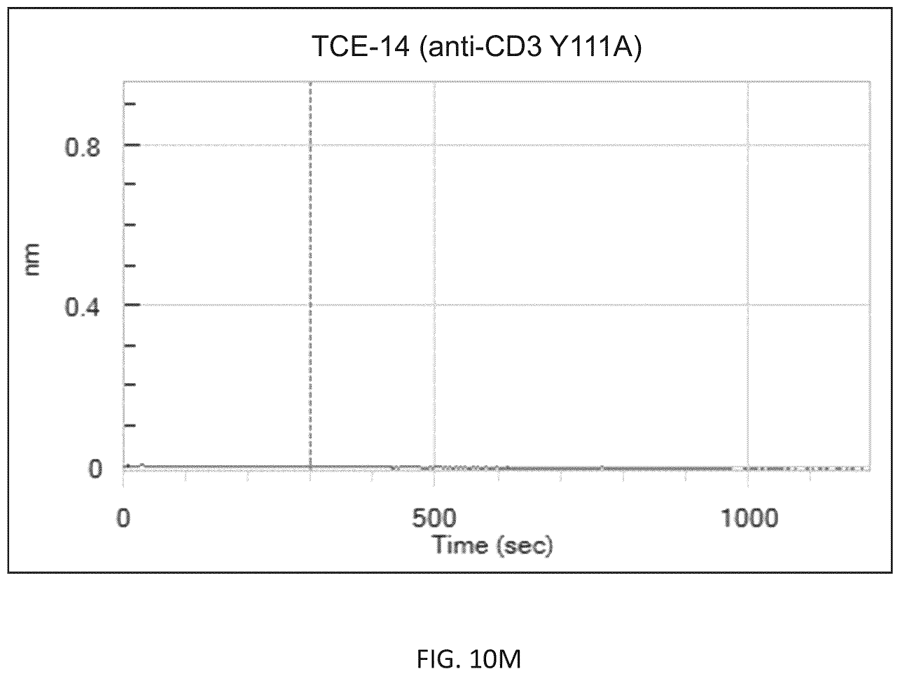

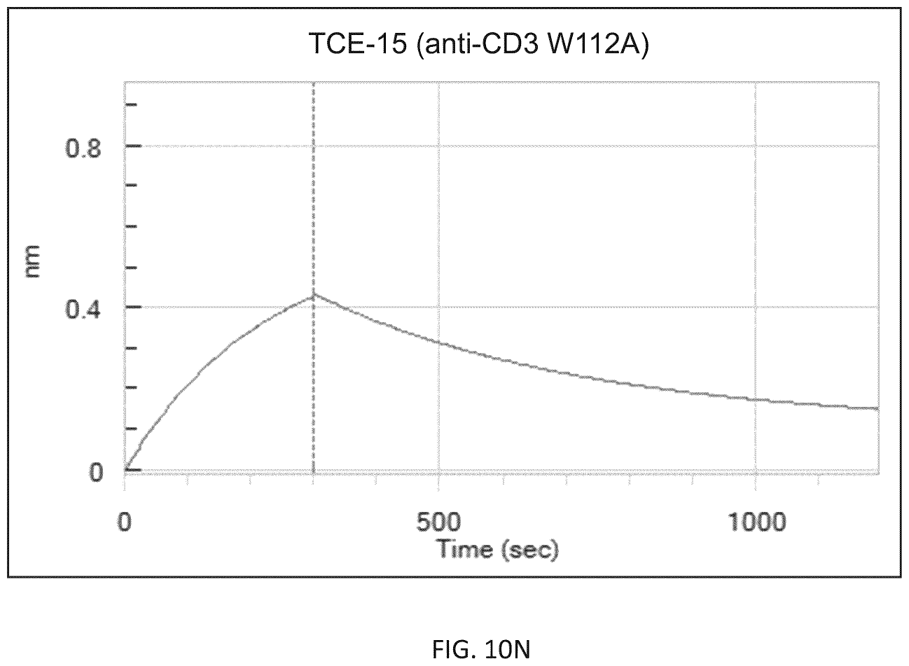

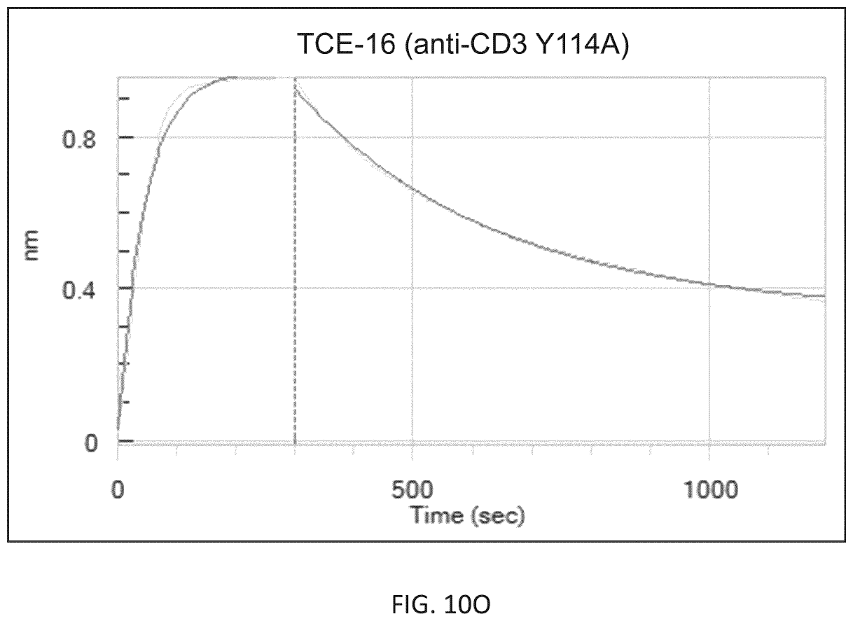

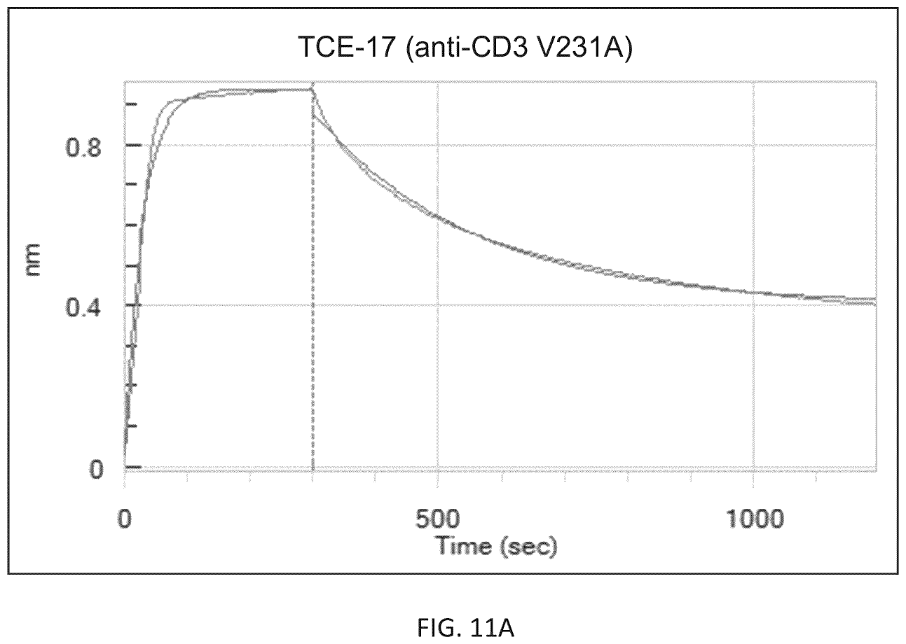

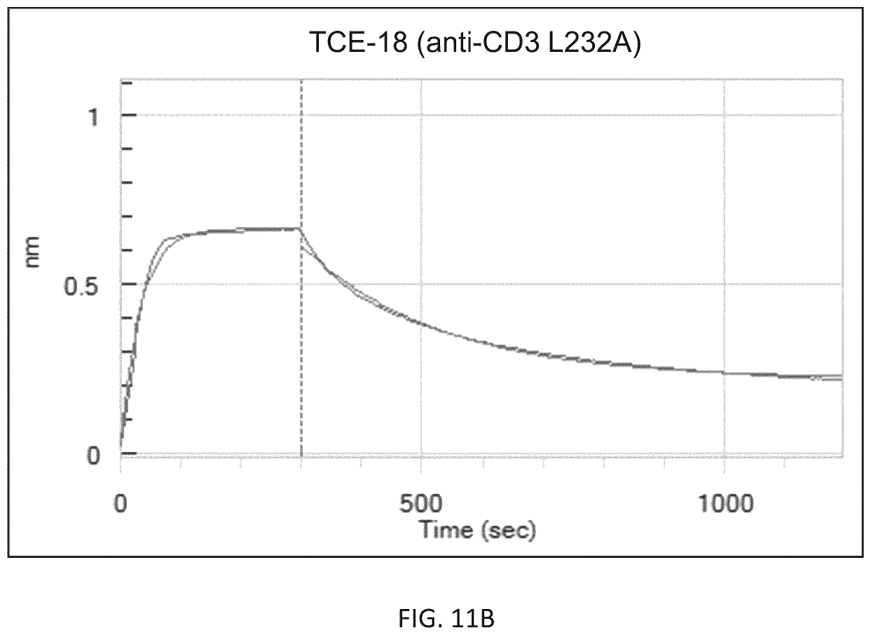

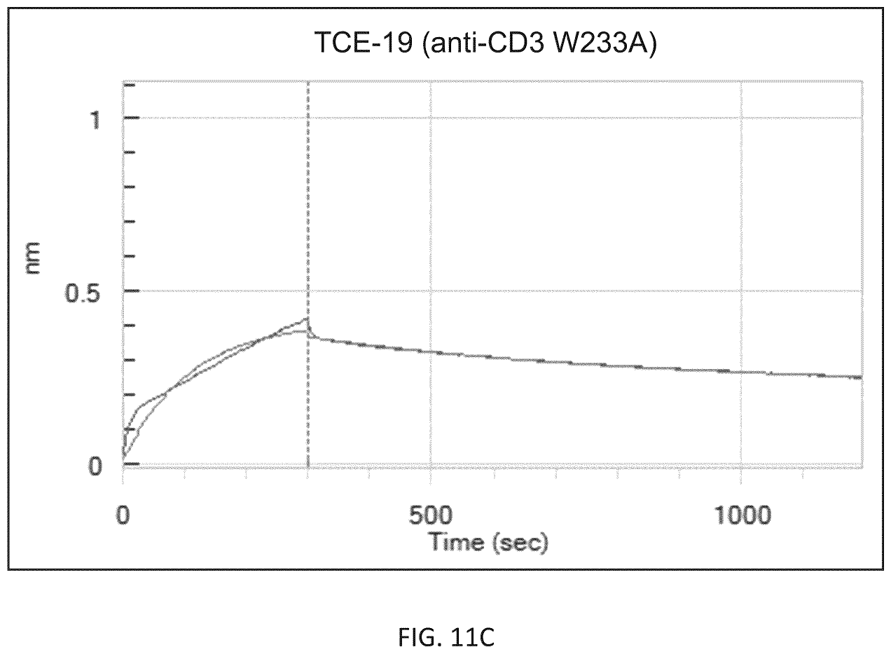

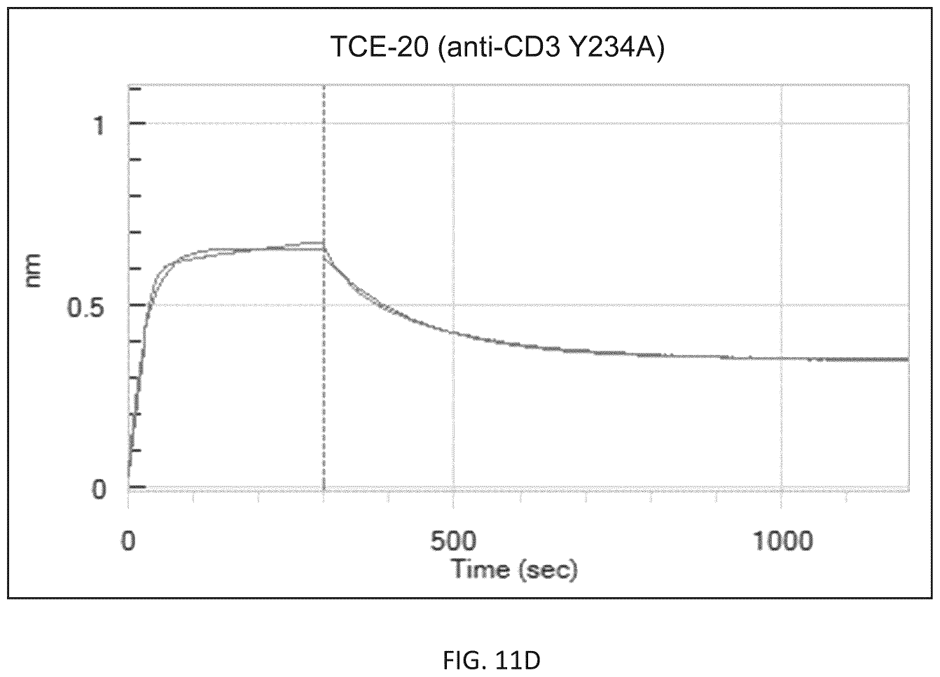

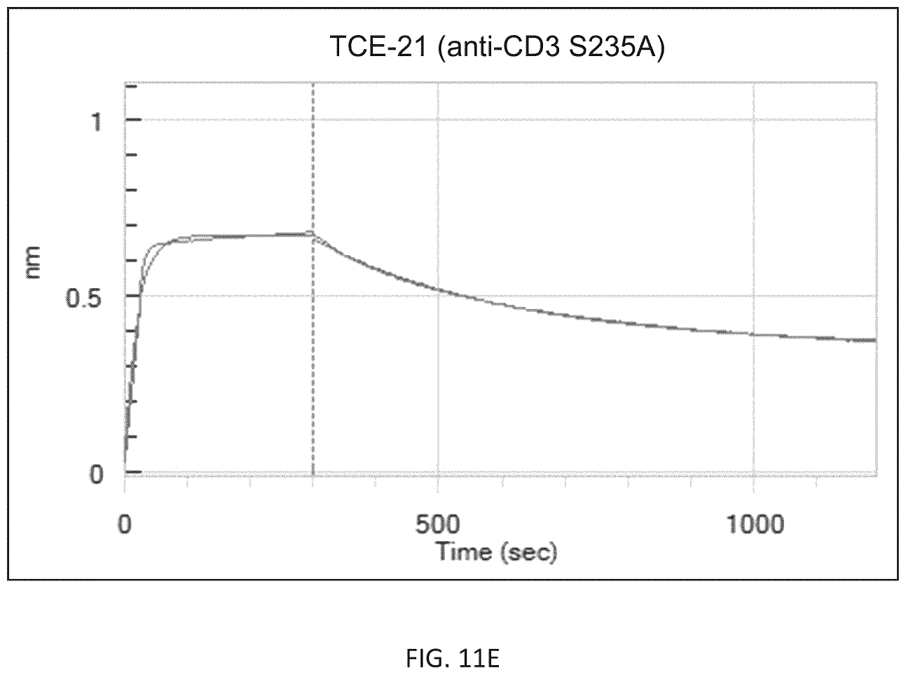

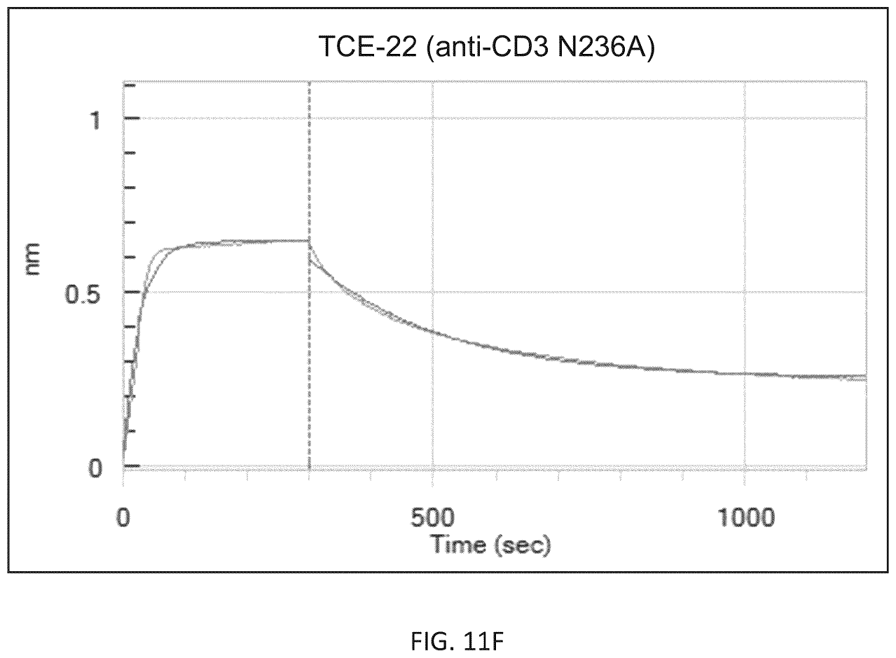

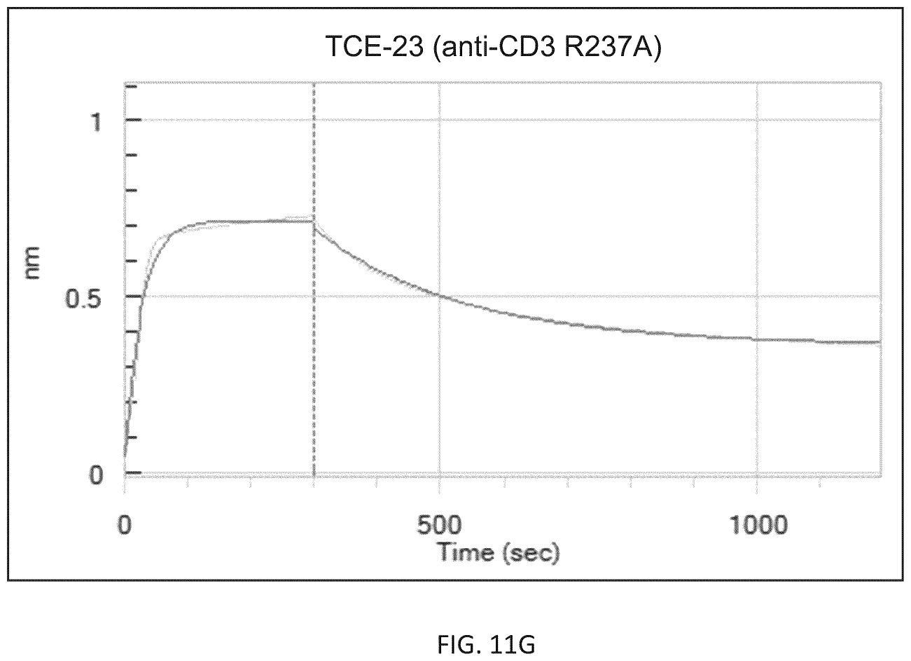

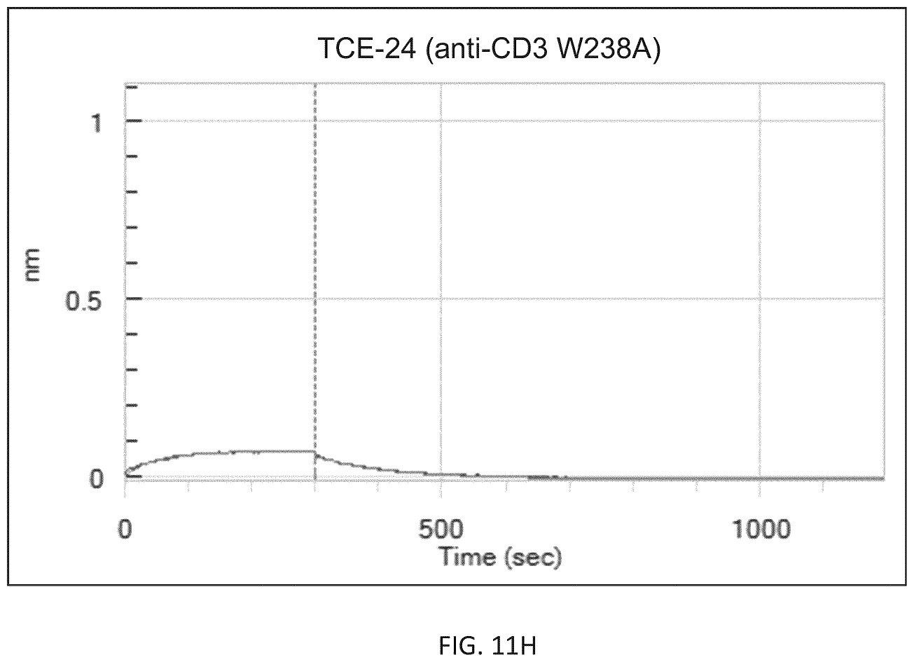

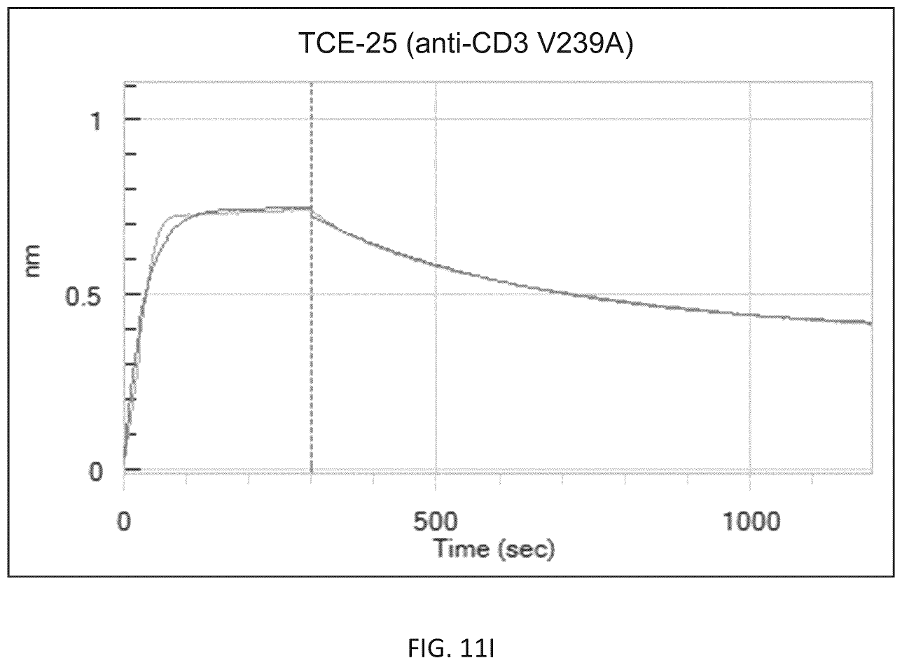

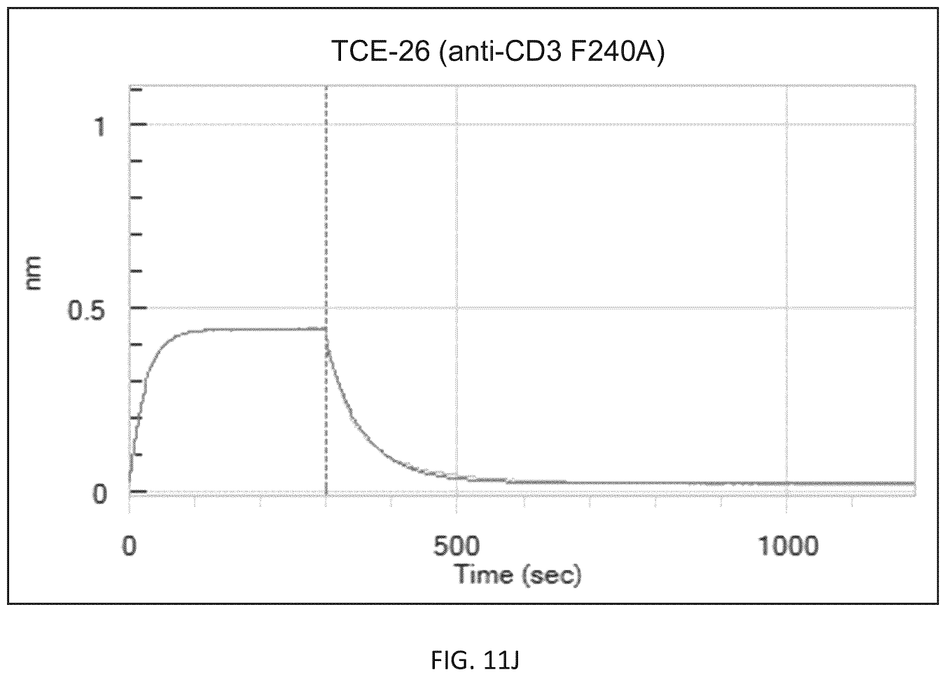

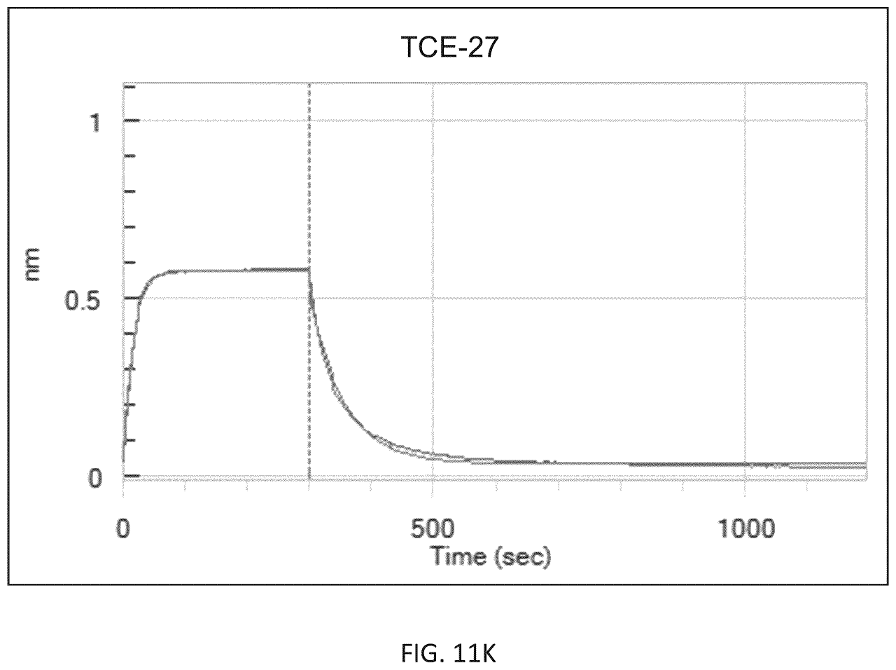

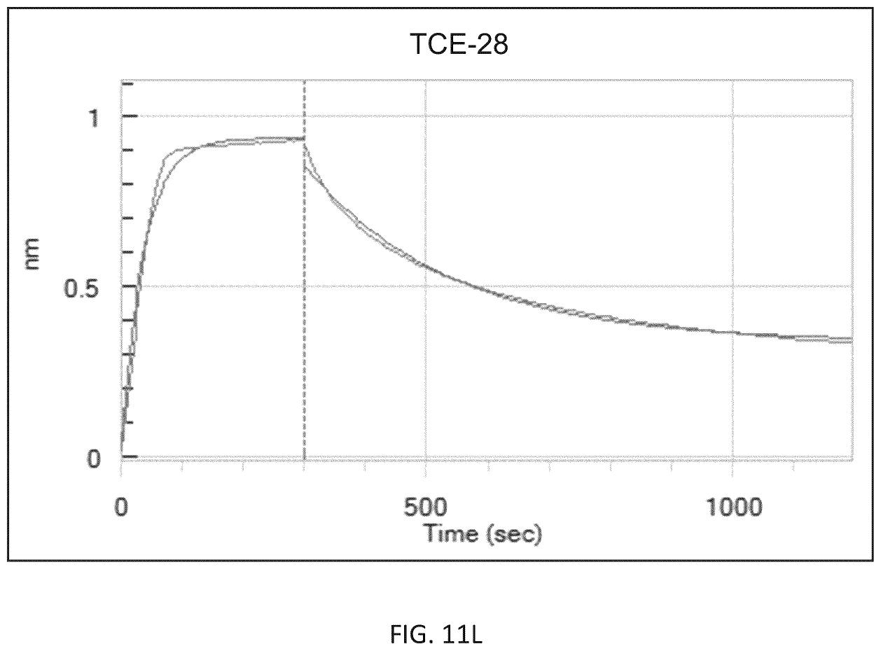

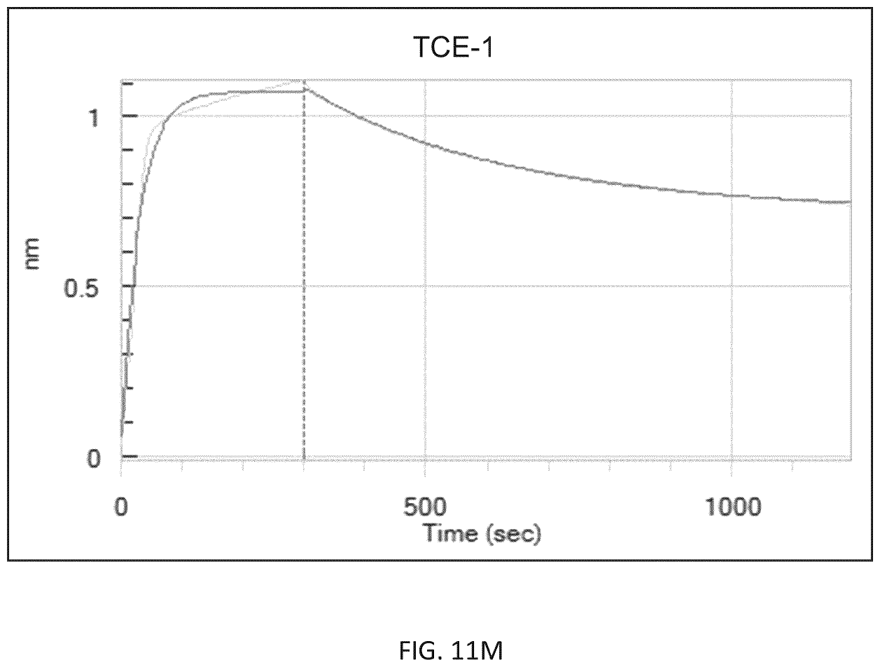

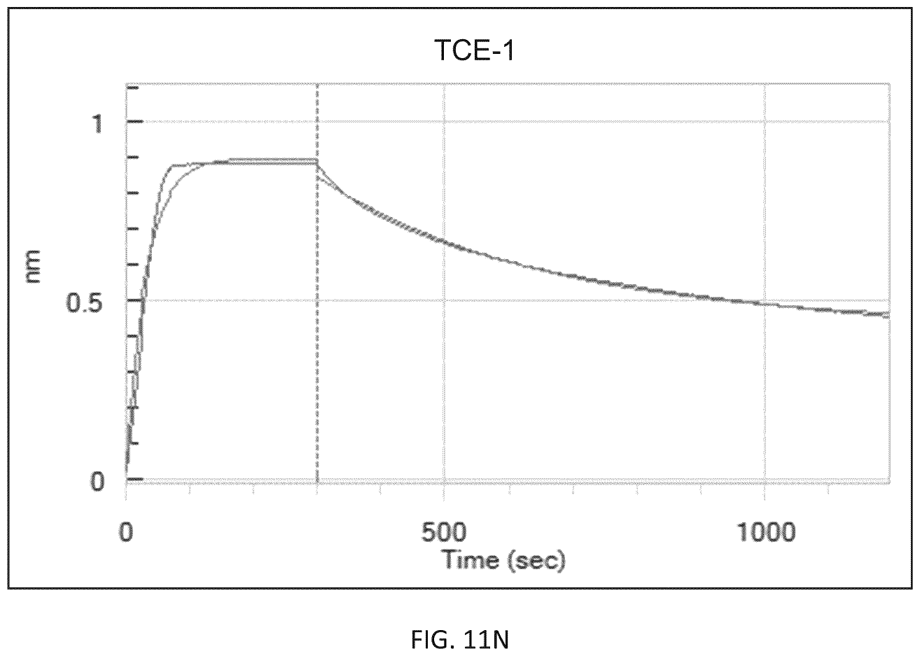

Example sensorgrams (partial fitting) for TCE-2 to TCE-16 (anti-CD3 CDR3 heavy chain mutants) are shown in FIGS. 10A-10O. Example sensorgrams (partial fitting) for TCE-17 to TCE-28 (including anti-CD3 CDR3 light chain mutants) and TCE-1 are shown in FIGS. 11A-11N. Calculated binding affinities (K_{D}s), association rates (kₒₙ), and dissociation rates (k_{dis}) for TCE-1 to TCE-28 from the BLI measurements are provided in Table 18.

TABLE 18

| Construct | | | | |
|---|---|---|---|---|
| description | K_{D} (M) | kₒₙ (1/M · s) | k_{dis} (1/s) | Response |
| TCE-2 | 6.06E−09 | 6.18E+05 | 3.75E−03 | 0.9106 |
| TCE-3 | 5.76E−08 | 2.08E+05 | 1.20E−02 | 0.7986 |
| TCE-4 | 2.37E−08 | 2.91E+05 | 6.90E−03 | 0.7208 |
| TCE-5 | 4.89E−09 | 7.52E+05 | 3.68E−03 | 0.9357 |
| TCE-6 | 1.68E−08 | 1.45E+05 | 2.43E−03 | 0.425 |
| TCE-7 | | | 4.42E−02 | 0.5478 |
| TCE-8 | 6.43E−09 | 5.72E+05 | 3.67E−03 | 0.9196 |
| TCE-9 | 3.83E−09 | 9.22E+05 | 3.53E−03 | 0.9385 |
| TCE-10 | 1.07E−08 | 4.47E+05 | 4.76E−03 | 0.909 |
| TCE-11 | 6.63E−09 | 7.48E+05 | 4.96E−03 | 0.9291 |
| TCE-12 | 2.25E−08 | 3.56E+05 | 8.00E−03 | 0.8159 |
| TCE-13 | | | 2.38E−02 | 0.5216 |
| TCE-14 | | | | −0.0009 |
| TCE-15 | 5.00E−08 | 4.65E+04 | 2.32E−03 | 0.4218 |
| TCE-16 | 7.86E−09 | 3.77E+05 | 2.97E−03 | 0.9557 |
| TCE-17 | 6.26E−09 | 6.22E+05 | 3.90E−03 | 0.939 |
| TCE-18 | 8.39E−09 | 5.23E+05 | 4.38E−03 | 0.6629 |
| TCE-19 | 1.23E−08 | 1.46E+05 | 1.79E−03 | 0.4142 |
| TCE-20 | 1.11E−08 | 6.26E+05 | 6.94E−03 | 0.6737 |
| TCE-21 | 3.41E−09 | 9.30E+05 | 3.17E−03 | 0.6816 |
| TCE-22 | 7.03E−09 | 6.72E+05 | 4.72E−03 | 0.6504 |
| TCE-23 | 6.51E−09 | 6.64E+05 | 4.32E−03 | 0.7284 |
| TCE-24 | 3.10E−08 | 2.50E+05 | 7.75E−03 | 0.0683 |
| TCE-25 | 4.76E−09 | 5.70E+05 | 2.71E−03 | 0.7422 |
| TCE-26 | 3.61E−08 | 4.90E+05 | 1.77E−02 | 0.444 |
| TCE-27 | 2.10E−08 | 8.62E+05 | 1.81E−02 | 0.5832 |
| TCE-28 | 9.11E−09 | 4.63E+05 | 4.22E−03 | 0.9306 |
| TCE- 1 | 4.91E−09 | 6.08E+05 | 2.98E−03 | 1.1051 |

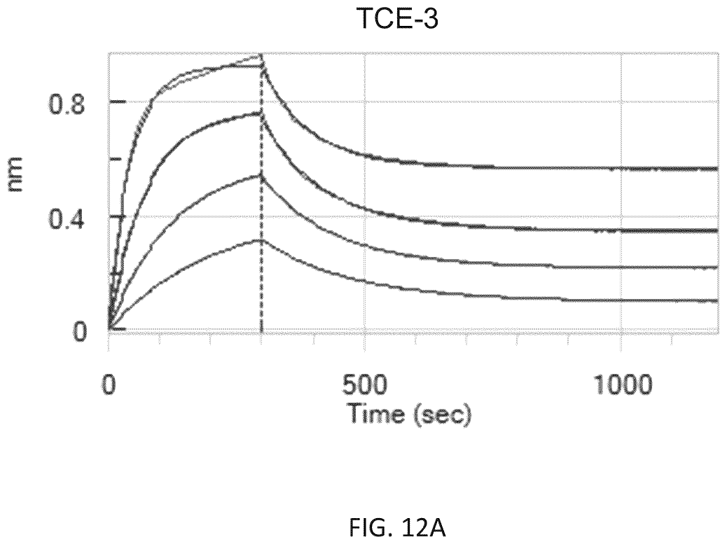

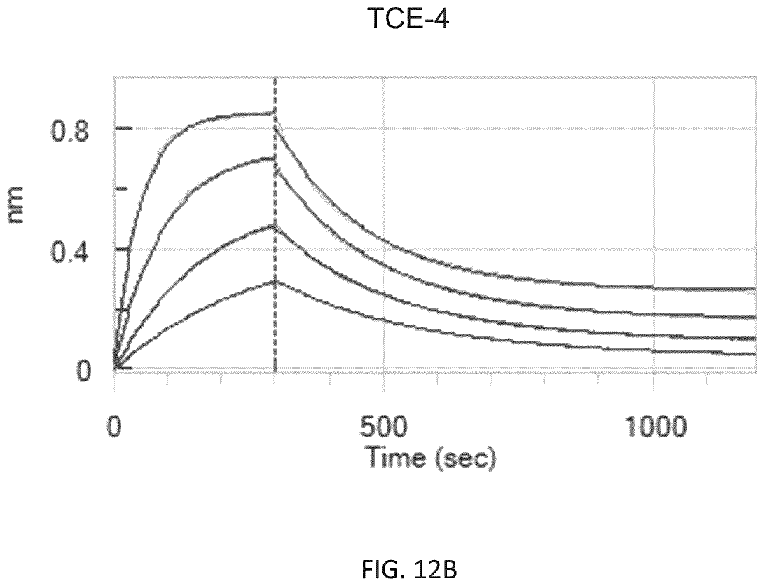

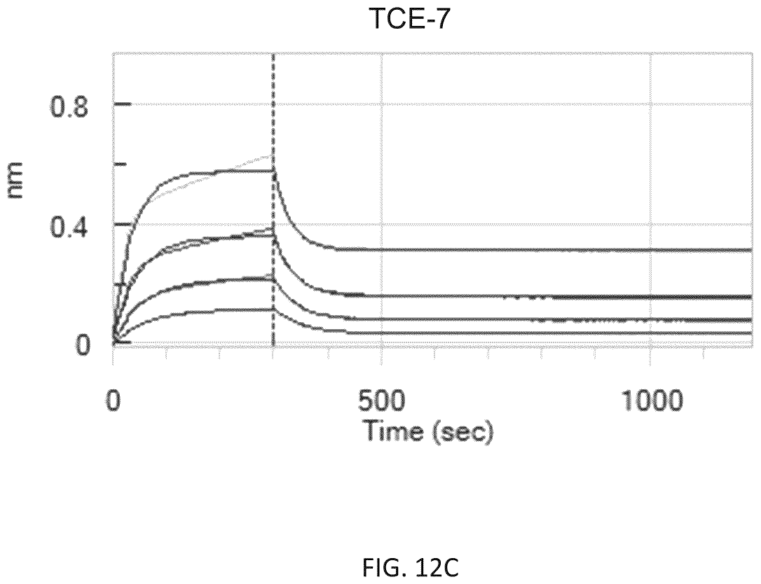

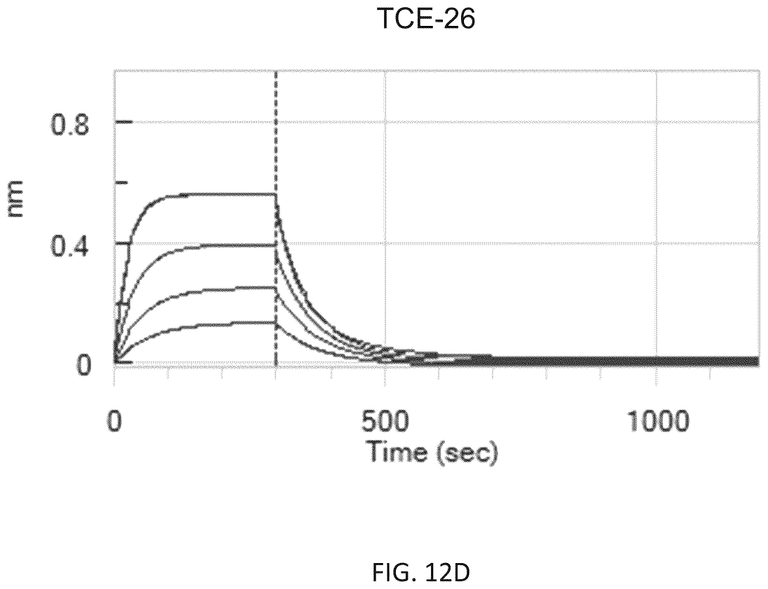

FIGS. 12A-12D show example BLI sensorgrams (local partial) for TCE-3 (FIG. 12A, anti-CD3 R100A mutant), TCE-4 (FIG. 12B, anti-CD3 H101A mutant), TCE-7 (FIG. 12C, anti-CD3 F104A mutant), and TCE-26 (FIG. 12D, anti-CD3 F240A mutant) binding to CD3ε using the experimental conditions and steps of Table 19. Calculated K_{D}s and kₒₙ and k_{dis} rates for TCE-3, TCE-4, TCE-7, and TCE-26 binding to CD3ε are provided in Table 20.

TABLE 19

| Step | Time |
|---|---|
| Sensor: SAX | — |
| Baseline: Octet buffer | 60 seconds |
| Loading: 10 nM human | 300 seconds |
| CD3ε-biotin | |
| Biocytin quench | 300 seconds |
| (100 μM) | |
| Baseline: Octet buffer | 300 seconds |
| Association: | 300 seconds |
| 50 nM TCE | |
| 25 nM TCE | |
| 12.5 nM TCE | |
| 6.25 nM TCE | |
| Dissociation: Octet buffer | 900 seconds |

TABLE 20

| Construct | Loading Sample | | | |
|---|---|---|---|---|
| description | ID | K_{D} (M) | kₒₙ (1/M · s) | k_{dis} (1/s) |
| TCE-3 | CD3e-bio [10 nM] | 4.42E−08 | 2.42E+05 | 1.07E−02 |
| TCE-4 | CD3e-bio [10 nM] | 2.09E−08 | 2.84E+05 | 5.93E−03 |
| TCE-7 | CD3e-bio [10 nM] | | | 3.36E−02 |
| TCE-26 | CD3e-bio [10 nM] | 3.46E−08 | 4.55E+05 | 1.57E−02 |

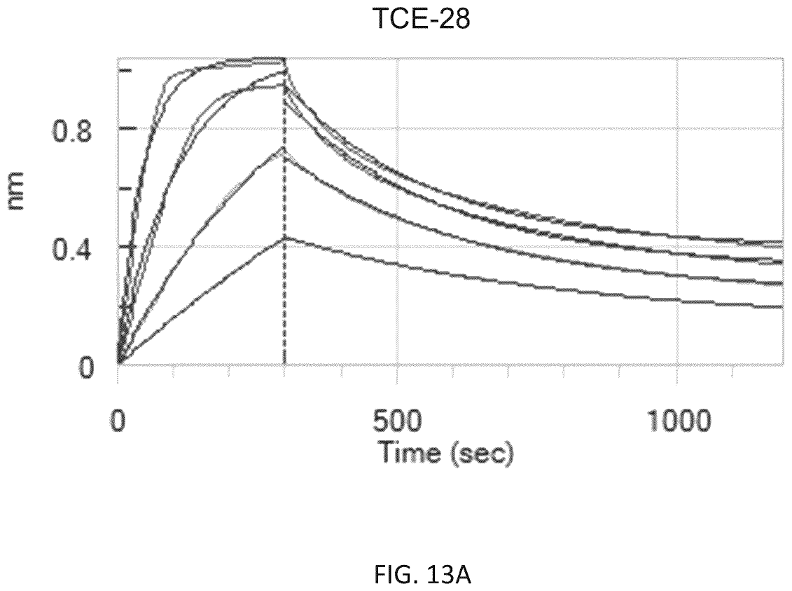

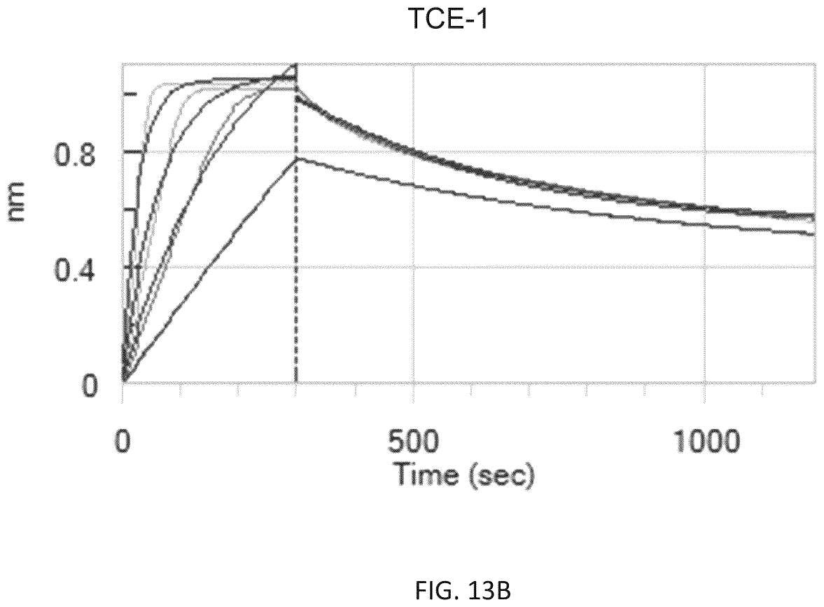

FIGS. 13A-13B show example BLI sensorgrams (local partial) for CD3ε binding by wild-type constructs TCE-28 (FIG. 13A) and TCE-1 (FIG. 13B) under the experimental conditions and steps of Table 19. Calculated K_{D}s and kₒₙ and k_{dis} rates for TCE-28 and TCE-1 binding to CD3ε are provided in Table 21.

TABLE 21

| Construct | Loading Sample | | | |
|---|---|---|---|---|
| description | ID | K_{D} (M) | kₒₙ (1/M · s) | k_{dis} (1/s) |
| TCE-28 | CD3e-bio [10 nM] | 1.07E−08 | 3.60E+05 | 3.85E−03 |
| TCE-1 | CD3e-bio [10 nM] | 5.30E−09 | 5.11E+05 | 2.71E−03 |

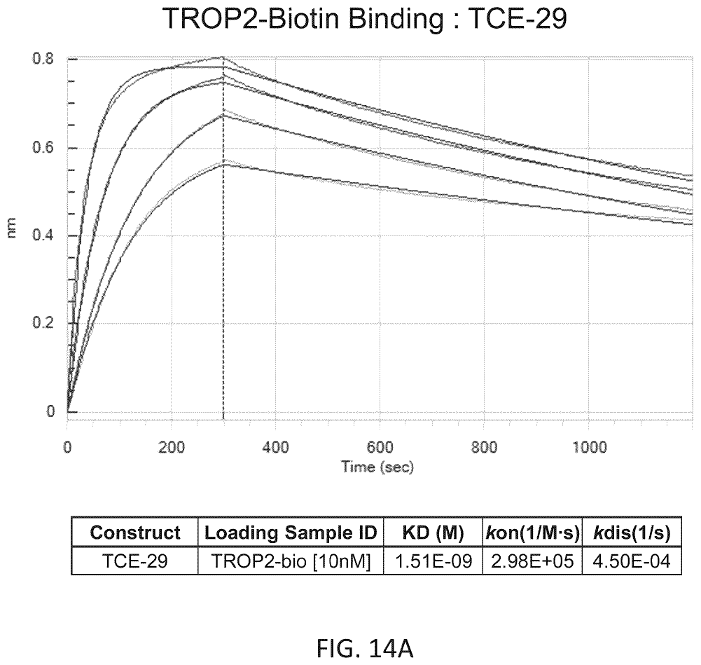

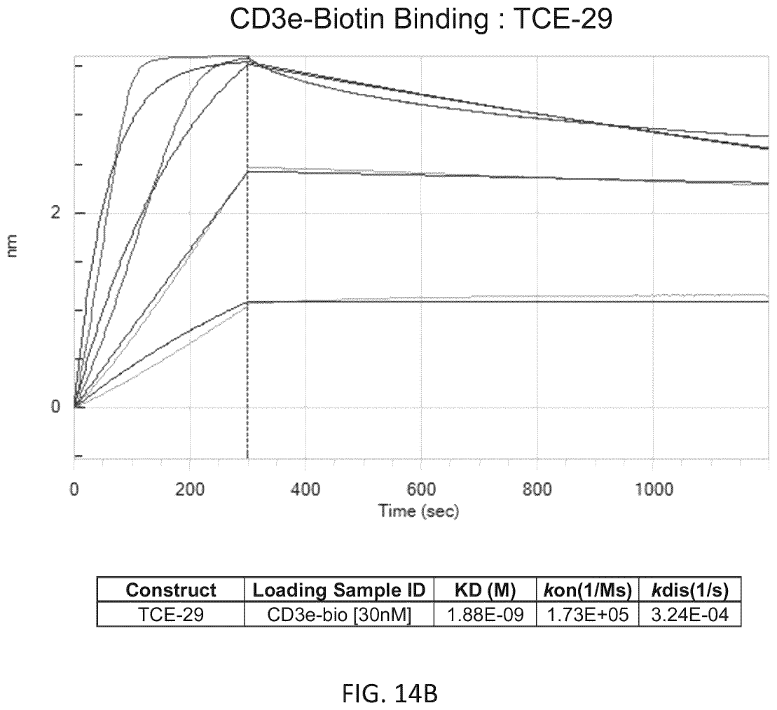

Example sensorgrams (local full) for TCE-29 (wild-type anti-CD3, anti-TROP2 F108A) binding to TROP2 (10 nM) and CD3ε (30 nM) as well as calculated K_{D}s, kₒₙ, and k_{dis} are shown in FIGS. 14A-14B. Exemplary experimental conditions and steps used for the measurements of FIGS. 14A-14B are provided in Table 22.

TABLE 22

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-29 | |
| 50 nM TCE-29 | |
| 25 nM TCE-29 | |
| 12.5 nM TCE-29 | |
| Dissociation: Octet buffer | 900 seconds |

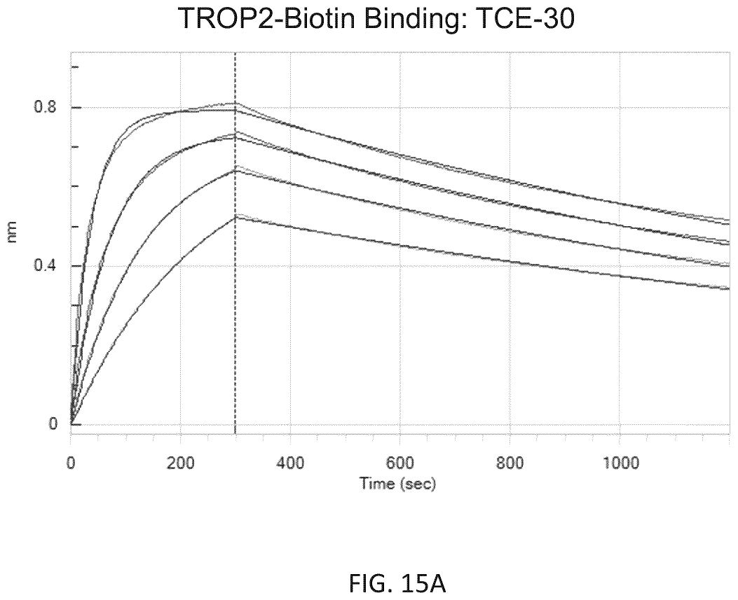

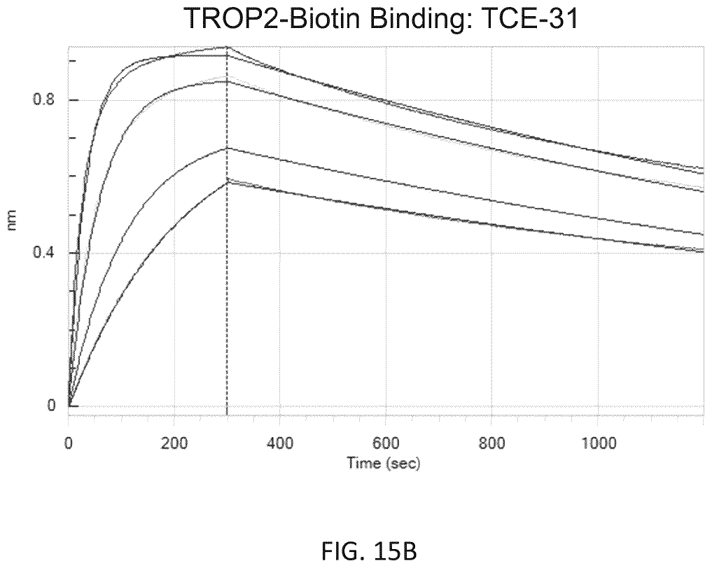

FIGS. 15A-15B show exemplary sensorgrams (Octet local full) for binding of double mutant TCE-30 (anti-CD3 H101A, anti-TROP2 F108A) and double mutant TCE-31 (anti-CD3 F104A, anti-TROP2 F108A) to TROP2 using the experimental conditions and steps of Table 23. Calculated K_{D}s and kₒₙ and k_{dis} rates for binding of TCE-30 and TCE-31 to TROP2 are provided in Table 24.

TABLE 23

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-30 | |
| 50 nM TCE-30 | |
| 25 nM TCE-30 | |
| 12.5 nM TCE-30 | |
| 100 nM TCE-31 | |
| 50 nM TCE-31 | |
| 25 nM TCE-31 | |
| 12.5 nM TCE-31 | |
| Dissociation: Octet buffer | 900 seconds |

TABLE 24

| Construct | | | | |
|---|---|---|---|---|
| description | Loading Sample ID | K_{D} (M) | kₒₙ (1/Mes) | k_{dis} (1/s) |
| TCE-30 | TROP2-bio [10 nM] | 1.56E−09 | 3.04E+05 | 4.75E−04 |
| TCE-31 | TROP2-bio [10 nM] | 1.21E−09 | 3.42E+05 | 4.14E−04 |

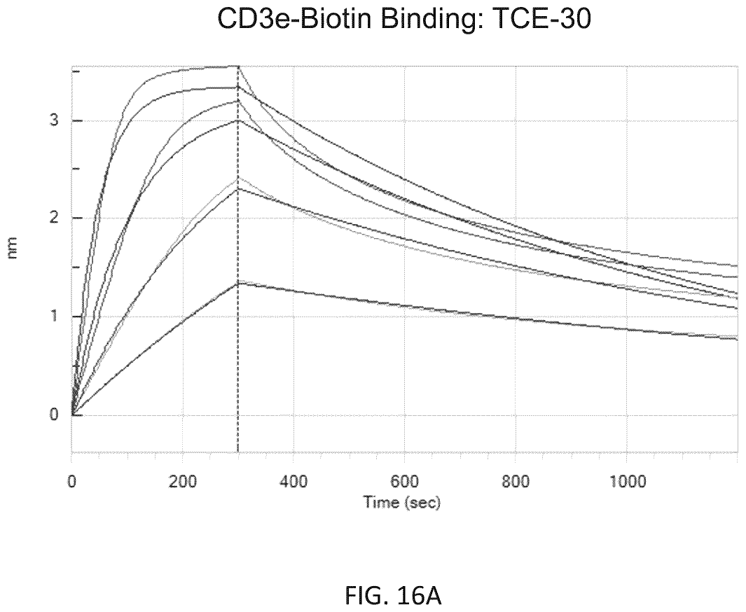

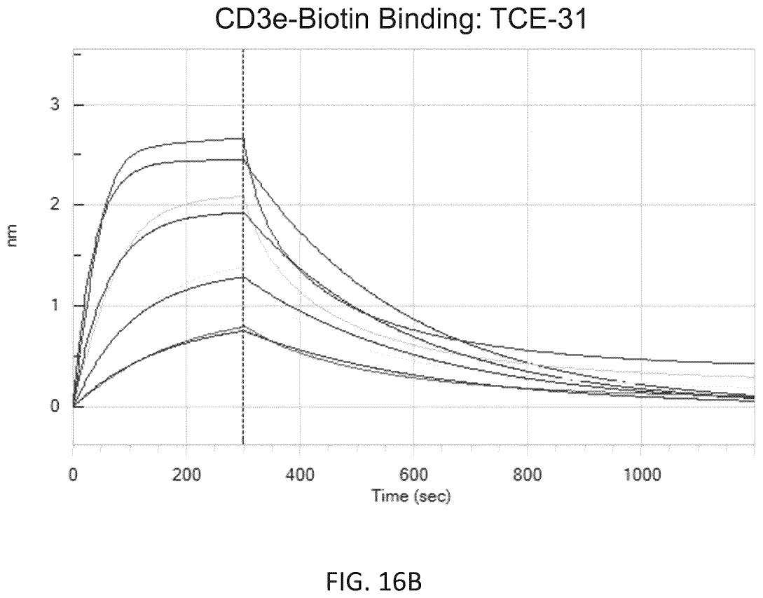

FIGS. 16A-16B show exemplary sensorgrams (Octet local full) for binding of double mutant TCE-30 (anti-CD3 H101A, anti-TROP2 F108A) and double mutant TCE-31 (anti-CD3 F104A, anti-TROP2 F108A) to CD3ε using the experimental conditions and steps of Table 23. Calculated K_{D}s and kₒₙ and k_{dis} rates for binding of TCE-30 and TCE-31 to CD3ε are provided in Table 25.

TABLE 25

| Construct | | | | |
|---|---|---|---|---|
| description | Loading Sample ID | K_{D} (M) | kₒₙ (1/Ms) | k_{dis} (1/s) |
| TCE-30 | CD3e-bio [30 nM] | 5.43E−09 | 2.04E+05 | 1.11E−03 |
| TCE-31 | CD3e-bio [30 nM] | 1.40E−08 | 2.47E+05 | 3.46E−03 |

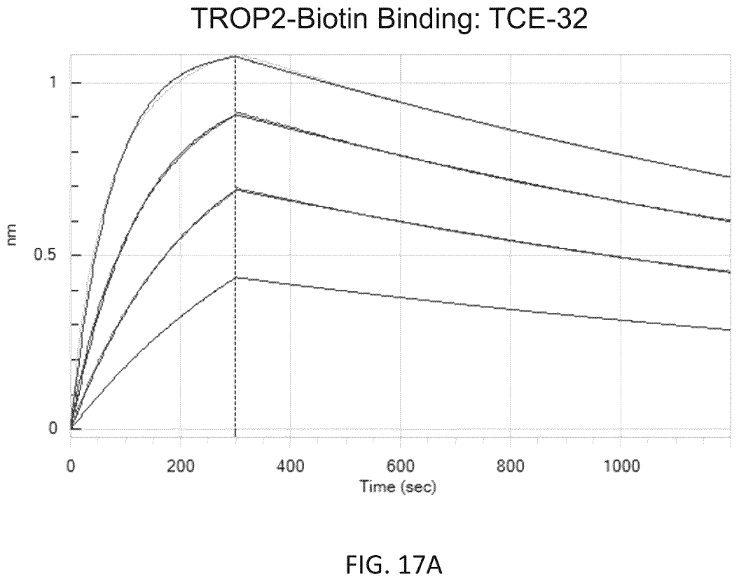

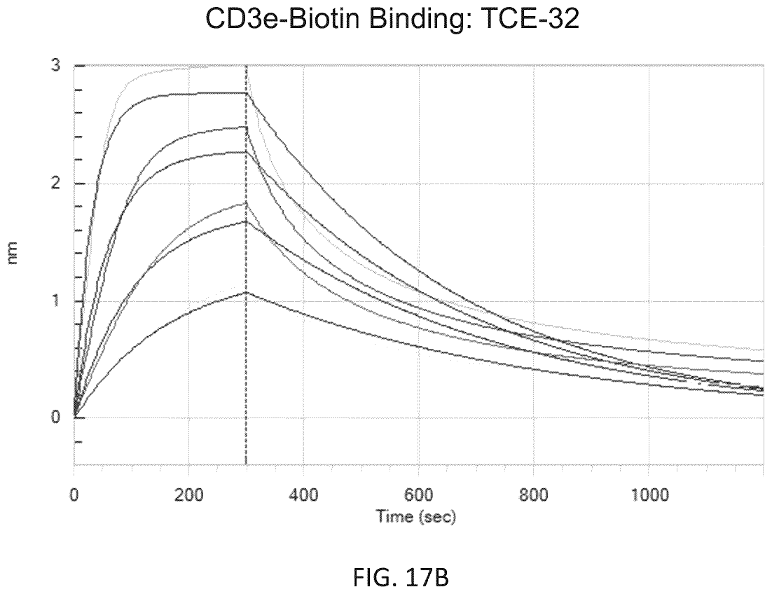

Example sensorgrams for double mutant TCE-32 (anti-CD3 F240A, anti-TROP2 F108A) binding to CD3ε are shown in FIGS. 17A-17B. Experimental conditions and steps used for the measurements are provided in Table 26. Calculated K_{D}s and kₒₙ and k_{dis} rates for binding of TCE-32 to TROP2 and CD3ε are provided in Table 27.

TABLE 26

| Step | Time |
|---|---|
| Sensor: SAX | — |
| Baseline: Octet buffer | 60 seconds |
| Loading: | 300 seconds |
| 10 nM TROP2-biotin | |
| 30 nM CD3e-biotin | |
| Biocytin quench | 300 seconds |
| (100 μM) | |
| Baseline: Octet buffer | 300 seconds |
| Association: | 300 seconds |
| 100 nM TCE-32 | |
| 50 nM TCE-32 | |
| 25 nM TCE-32 | |
| 12.5 nM TCE-32 | |
| Dissociation: Octet buffer | 900 seconds |

TABLE 27

| Construct | | | | |
|---|---|---|---|---|
| description | Loading Sample ID | K_{D} (M) | kₒₙ (1/M.s) | k_{dis} (1/s) |
| TCE-32 | TROP2-bio [10 nM] | 2.92E−09 | 1.62E+05 | 4.73E−04 |
| TCE-32 | CD3e-bio [30 nM] | 9.12E−09 | 2.90E+05 | 2.64E−03 |

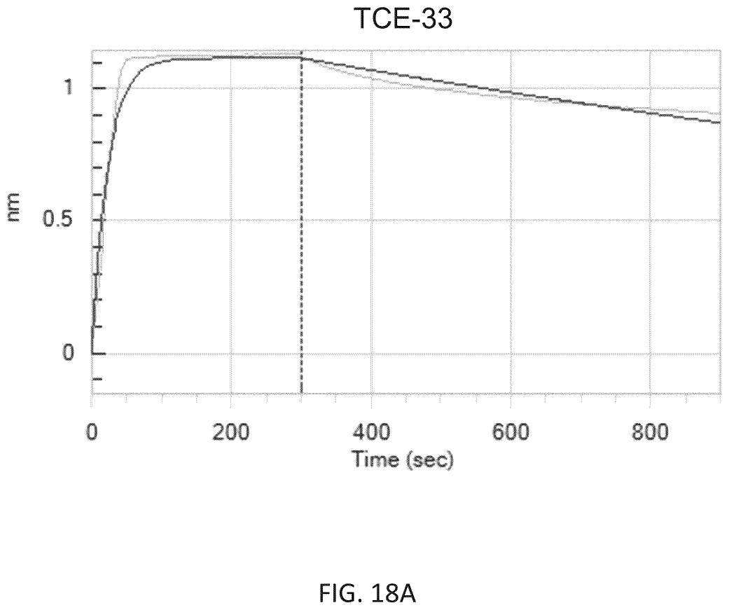

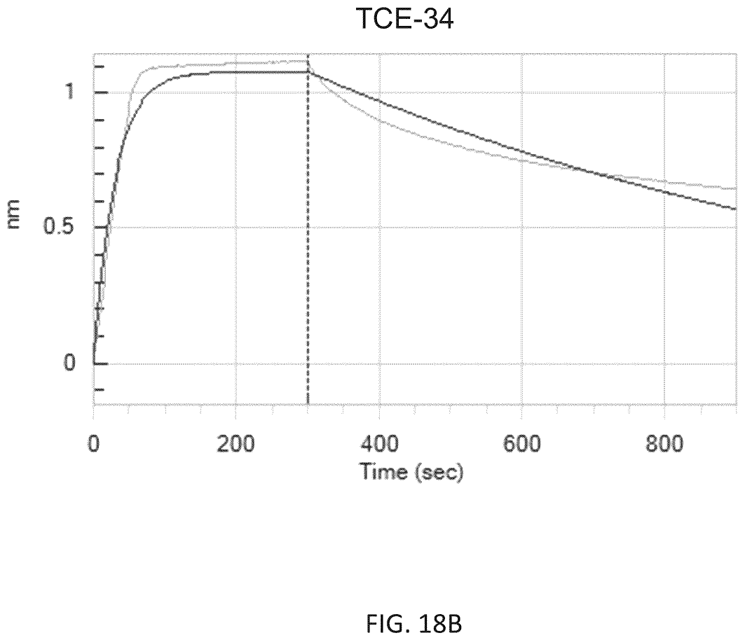

Exemplary sensorgrams for TCE-33 (anti-TROP2 F108A mutant) and TCE-34 (anti-TROP2 F108A mutant) binding to CD3ε are shown in FIGS. 18A-18B. Exemplary experimental conditions and steps used for the measurements of FIGS. 18A-18B are provided in Table 28, and calculated K_{D}s and kₒₙ and k_{dis} rates for TCE-33 and TCE-34 binding to CD3ε are provided in Table 29.

TABLE 28

| Step | Time |
|---|---|
| Baseline: Octet buffer | 60 seconds |
| Loading: | 300 seconds |
| 10 nM human CD3- | |
| biotin | |
| Biocytin quench | 300 seconds |
| (100 μM) | |
| Baseline: Octet buffer | 300 seconds |
| Association: | 300 seconds |
| 50 nM TCE | |
| Dissociation: Octet buffer | 900 seconds |

TABLE 29

| Construct | | K_{D} | kₒₙ | k_{dis} |
|---|---|---|---|---|
| description | Loading Sample ID | (M) | (1/M · s) | (1/s) |
| TCE-33 | Biotinylated CD3e | 4.67E−10 | 8.85E+05 | 4.14E−04 |
| TCE-34 | Biotinylated CD3e | 1.62E−09 | 6.59E+05 | 1.07E−03 |

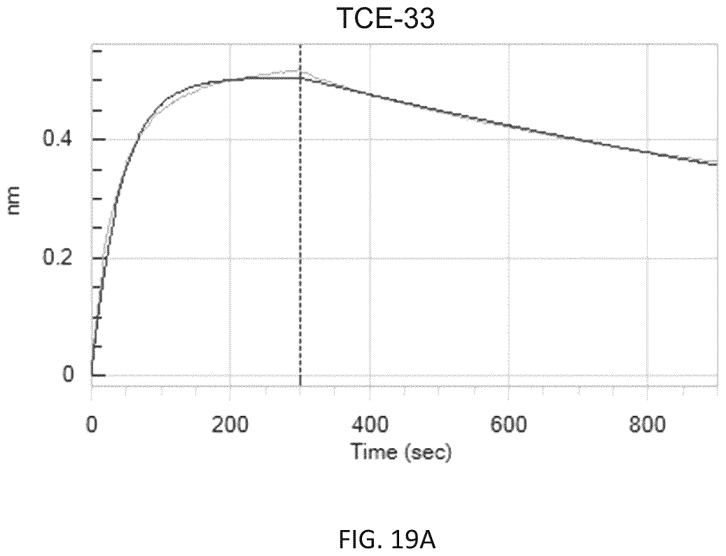

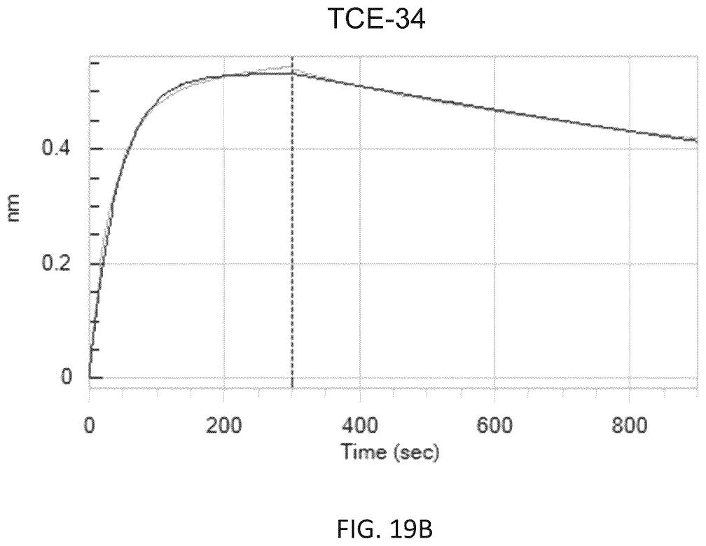

Exemplary sensorgrams for TCE-33 (anti-TROP2 F108A mutant) and TCE-34 (anti-TROP2 F108A mutant) binding to TROP2 are shown in FIGS. 19A-19B. Exemplary experimental conditions and steps used for the measurements of FIGS. 19A-19B are provided in Table 30, and calculated K_{D}s and kₒₙ and k_{dis} rates for TCE-33 and TCE-34 binding to TROP2 are provided in Table 31.

TABLE 30

| Step | Time |
|---|---|
| Baseline: Octet buffer | 60 seconds |
| Loading: | 300 seconds |
| 10 nM human TROP2- | |
| biotin | |
| Biocytin quench (100 | 300 seconds |
| μM) | |
| Baseline: Octet buffer | 300 seconds |
| Association: | 300 seconds |
| 50 nM TCE | |
| Dissociation: Octet buffer | 900 seconds |

TABLE 31

| Construct | | K_{D} | kₒₙ | k_{dis} |
|---|---|---|---|---|
| description | Loading Sample ID | (M) | (1/M · s) | (1/s) |
| TCE-33 | Biotinylated TROP2 | 1.22E−09 | 4.74E+05 | 5.80E−04 |
| TCE-34 | Biotinylated TROP2 | 8.81E−10 | 4.76E+05 | 4.19E−04 |

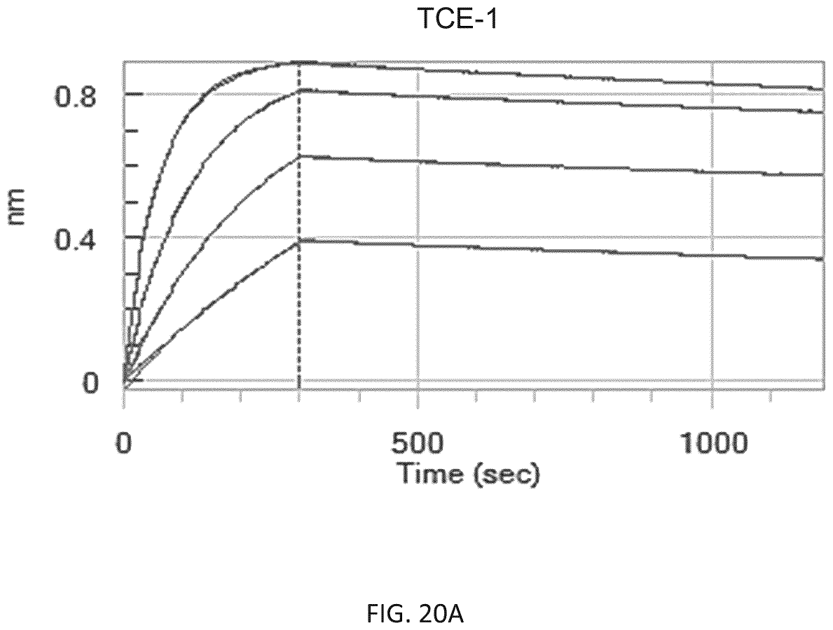

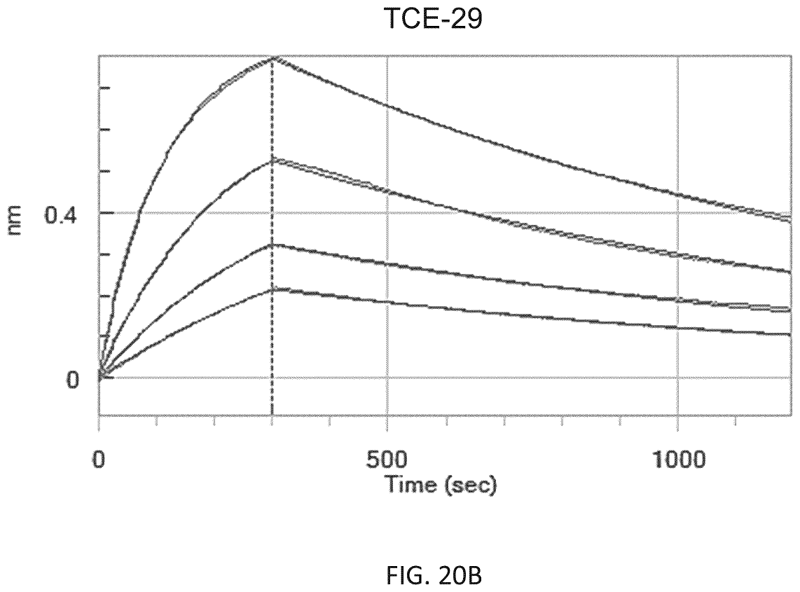

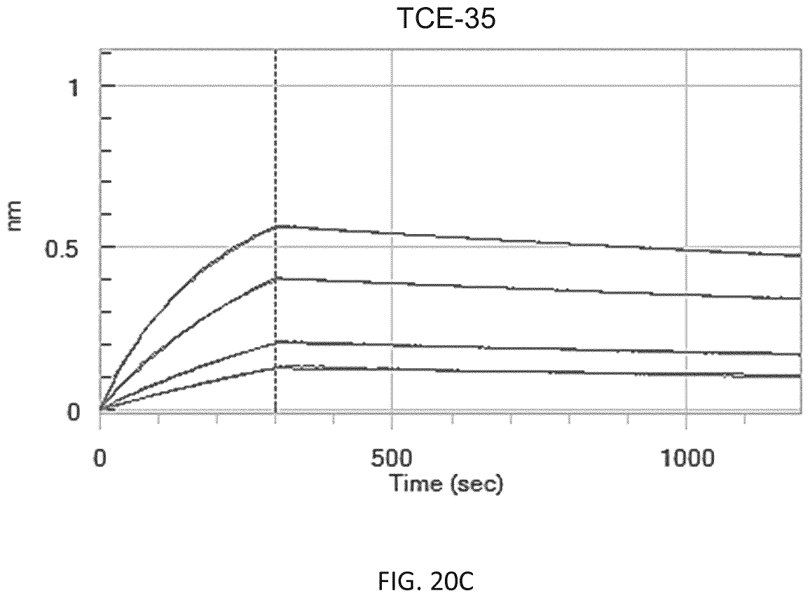

Exemplary sensorgrams for TCE-1 (wild-type anti-CD3, wild-type anti-TROP2), TCE-29 (anti-TROP2 F108A mutant), and TCE-35 (anti-TROP2 D109A mutant) binding to TROP2 are shown in FIGS. 20A-20C. Exemplary experimental conditions and steps used for the measurements of FIGS. 20A-20C are provided in Table 32, and calculated K_{D}s and kₒₙ and k_{dis} rates are provided in Table 33.

TABLE 32

| Step | Time |
|---|---|
| Sensor: SAX | — |
| Baseline: Octet buffer | 60 seconds |
| Loading: Human TROP2- | 300 seconds |
| biotin [10 nM] | |
| Biocytin quench (100 | 300 seconds |
| μM) | |
| Baseline: Octet buffer | 300 seconds |
| Association: | 300 seconds |
| 50 nM, 25 nM, | |
| 12.5 nM, 6.25 nM | |
| TCE | |
| Dissociation: Octet buffer | 600 seconds |

TABLE 33

| Construct | Loading | | K_{D} | | | | |
|---|---|---|---|---|---|---|---|
| description | Sample ID | K_{D} (M) | Error | kₒₙ (1/M · s) | k_{dis} (1/s) | Full R² | Full X² |
| TCE-29 | hTROP2- | 4.94E−09 | 1.28E−11 | 1.62E+05 | 7.97E−04 | 0.9993 | 0.1112 |
| | Biotin | | | | | | |
| TCE-35 | hTROP2- | 1.91E−09 | 8.06E−12 | 1.04E+05 | 1.99E−04 | 0.9996 | 0.0558 |
| | Biotin | | | | | | |
| TCE-1 | hTROP2- | 2.60E−10 | 1.72E−12 | 3.79E+05 | 9.85E−05 | 0.999 | 0.1847 |
| | Biotin | | | | | | |

### Example 3: Sequence Activity Relationships

Data summarizing CD3ε binding parameters obtained from the above equilibrium and kinetic binding studies are provided in Table 34 (anti-CD3 CDR3 LC alanine mutated constructs) and Tables 35-36 (anti-CD3 CDR3 HC alanine mutated constructs). The tables also classify each alanine mutation in the CDR3 LC and CDR3 HC regions as tolerated (T), moderately tolerated (MT), or not tolerated (NT). Alanine mutations at residues which resulted in significantly weakened binding relative to the non-mutated TCE construct TCE-1 were characterized as NT, and those residues are considered key residues for CD3ε binding. Alanine mutations at residues which showed comparable binding parameters to TCE-1 were characterized as T and are considered non-critical for CD3ε binding. Alanine mutations which at residues resulted in somewhat weakened binding relative to TCE-1 were characterized as MT.

As can be seen from Table 34, alanine mutations at residues W233 and W238 of the anti-CD3 CDR3 LC were not tolerated indicating that these residues are important for binding to CD3. Additionally, Tables 35-36 demonstrate that alanine mutations at N103, F104, Y111, and W112 of the anti-CD3 CDR3 HC were not tolerated indicating that these residues are also important for binding to CD3.

TABLE 34
CD3ε Binding and SAR Data for anti-CD3 CDR3 LC Alanine Scanning Mutants

| | TCE1, | TCE | TCE | TCE | TCE | TCE | TCE | TCE | TCE | TCE | TCE |
|---|---|---|---|---|---|---|---|---|---|---|---|
| Construct | wt | 17 | 18 | 19 | 20 | 21 | 22 | 23 | 24 | 25 | 26 |
| CD3 K_{D} | 4.91 | 6.26 | 8.39 | 12.30 | 11.10 | 3.41 | 7.03 | 6.51 | 31.00 | 4.76 | 36.10 |
| (nM) | | | | | | | | | | | |
| CD3 t_{1/2} | 3.88 | 2.96 | 2.64 | 6.45 | 1.66 | 3.64 | 2.45 | 2.67 | 1.49 | 4.26 | 0.65 |
| (min) | | | | | | | | | | | |
| CD3 Octet | 1.11 | 0.94 | 0.66 | 0.41 | 0.67 | 0.68 | 0.65 | 0.73 | 0.07 | 0.74 | 0.44 |
| response | | | | | | | | | | | |
| CD3 EC₅₀ | 0.17 | 0.17 | 0.20 | 1.81 | 0.18 | 0.16 | 0.18 | 0.20 | 8.65 | 0.19 | 0.60 |
| ELISA | | | | | | | | | | | |
| (nM) | | | | | | | | | | | |
| Fold shift | — | 1 | 1.18 | 10.6 | 1.05 | 0.94 | 1.05 | 1.18 | 50.9 | 1.12 | 3.53 |
| in EC₅₀ | | | | | | | | | | | |
| relative to | | | | | | | | | | | |
| wt | | | | | | | | | | | |
| CDR3 LC | — | V | L | W | Y | S | N | R | W | V | F |
| Ala scan | | | | | | | | | | | |
| Amino | — | 231 | 232 | 233 | 234 | 235 | 236 | 237 | 238 | 239 | 240 |
| acid | | | | | | | | | | | |
| position | | | | | | | | | | | |
| Mutation | — | T | T | NT | T | T | T | T | NT | T | MT |
| tolerated? | | | | | | | | | | | |

TABLE 35
CD3ε Binding and SAR Data for anti-CD3 CDR3 HC Alanine Scanning Mutants

| | TCE1, | not | | | | | | | |
|---|---|---|---|---|---|---|---|---|---|
| Construct | wt | mutated | TCE2 | TCE3 | TCE4 | TCE5 | TCE6 | TCE7 | TCE8 |
| CD3 K_{D} | 4.91 | — | 6.1 | 57.6 | 23.7 | 4.9 | 16.8 | | 6.4 |
| (nM) | | | | | | | | | |
| CD3 t_{1/2} | 3.88 | — | 3.1 | 1.0 | 1.7 | 3.1 | 4.8 | 0.3 | 3.1 |
| (min) | | | | | | | | | |
| CD3 Octet | 1.11 | — | 0.91 | 0.80 | 0.72 | 0.94 | 0.43 | 0.55 | 0.92 |
| response | | | | | | | | | |
| CD3 EC₅₀ | 0.17 | — | 0.20 | 0.28 | 0.27 | 0.22 | 3.13 | 1.14 | 0.19 |
| ELISA | | | | | | | | | |
| (nM) | | | | | | | | | |
| Fold shift | — | — | 1.18 | 1.65 | 1.59 | 1.29 | 18.4 | 6.71 | 1.12 |
| in EC₅₀ | | | | | | | | | |
| relative to | | | | | | | | | |
| wt | | | | | | | | | |
| CDR3 HC | — | A | V | R | H | G | N | F | G |
| Ala scan | | | | | | | | | |
| Amino | — | — | 99 | 100 | 101 | 102 | 103 | 104 | 105 |
| acid | | | | | | | | | |
| position | | | | | | | | | |
| Mutation | — | — | T | MT | MT | T | NT | NT | T |
| tolerated? | | | | | | | | | |

TABLE 36
CD3ε Binding and SAR Data for anti-CD3 CDR3 HC Alanine Scanning Mutants

| | | | | | | | | not | |
|---|---|---|---|---|---|---|---|---|---|
| Construct | TCE9 | TCE10 | TCE11 | TCE12 | TCE13 | TCE14 | TCE15 | mutated | TCE16 |
| CD3 K_{D} | 3.8 | 10.7 | 6.6 | 22.5 | | | 50.0 | — | 7.9 |
| (nM) | | | | | | | | | |
| CD3 t_{1/2} | 3.3 | 2.4 | 2.3 | 1.4 | 0.5 | | 5.0 | — | 3.9 |
| (min) | | | | | | | | | |
| CD3 Octet | 0.94 | 0.91 | 0.93 | 0.82 | 0.52 | 0.00 | 0.42 | — | 0.96 |
| response | | | | | | | | | |
| CD3 EC₅₀ | 0.20 | 0.22 | 0.14 | 0.24 | 0.56 | 732.60 | 0.56 | — | 0.20 |
| ELISA | | | | | | | | | |
| (nM) | | | | | | | | | |
| Fold shift | 1.18 | 1.29 | 0.82 | 1.41 | 3.29 | >1000 | 3.29 | — | 1.18 |
| in EC₅₀ | | | | | | | | | |
| relative to | | | | | | | | | |
| wt | | | | | | | | | |
| CDR3 HC | N | S | Y | I | S | Y | W | A | Y |
| Ala scan | | | | | | | | | |
| Amino | 106 | 107 | 108 | 109 | 110 | 111 | 112 | — | 114 |
| acid | | | | | | | | | |
| position | | | | | | | | | |
| Mutation | T | T | T | MT | MT | NT | NT | — | T |
| tolerated? | | | | | | | | | |

### Example 4: Tumor Cell Killing with TROP2 TCEs

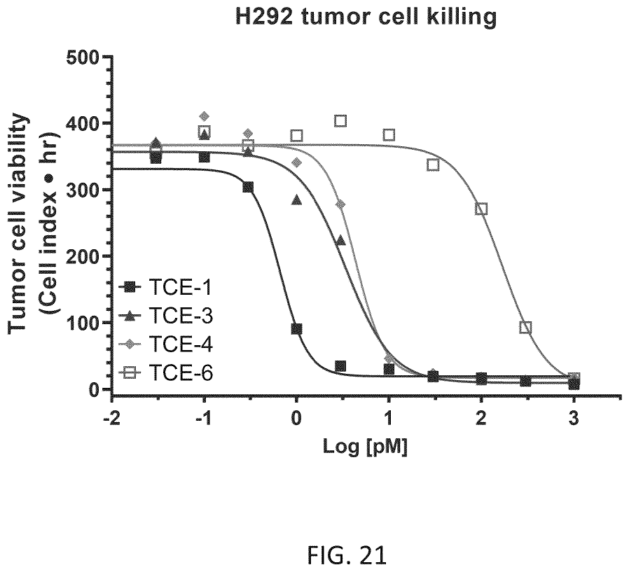

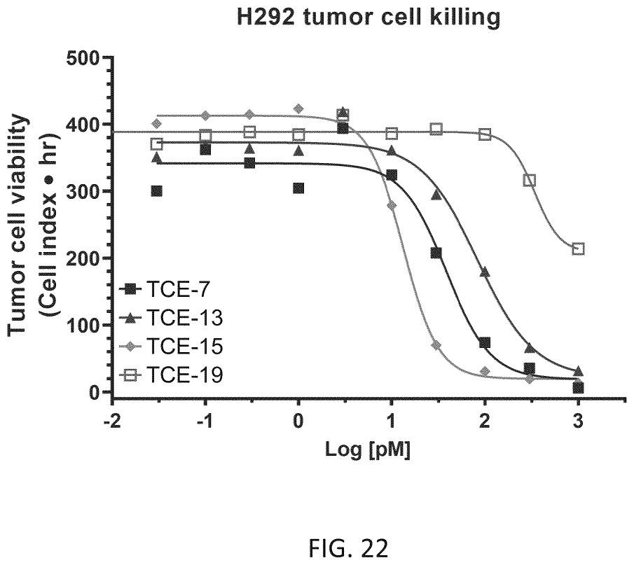

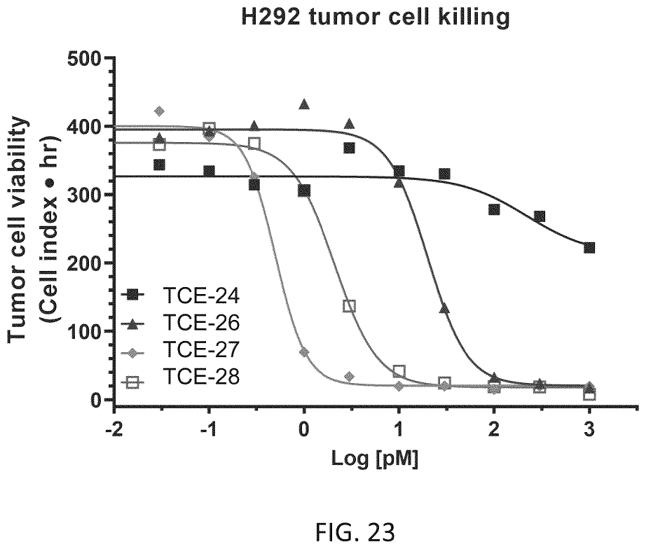

TCEs were evaluated in a functional in vitro tumor cell killing assay using H292, HCT116, or HEK293 cell lines. Recombinant HEK293 cell lines were engineered to express human or cynomolgus monkey TROP2 on their surface. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight. The following day, TCEs titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of TCE where the concentration required to reduce the tumor growth by 50% (EC₅₀) was calculated using Graphpad Prism software. Data plots of tumor cell viability vs. TCE concentration are shown in FIG. 21 for TCE-1, TCE-3, TCE-4, and TCE-6 mediated killing of H292 tumor cells. FIG. 22 shows data plots of tumor cell viability vs. TCE concentration for TCE-7, TCE-13, TCE-15, and TCE-19 mediated killing of H292 tumor cells. FIG. 23 shows data plots for H292 tumor cell viability vs. TCE concentration for TCE-24, TCE-26, TCE-27, and TCE-28. The EC₅₀s for tumor cell killing of H292 cells by the different TCEs are provided in Tables 37-39.

TABLE 37
TCE Cytotoxicity EC₅₀s (H292 Cells) - Mutated CDRH3

| Anti-CD3 scFv | | |
|---|---|---|
| CDRH3 | | H292 Cytotox |
| mutation | TCE Construct | EC₅₀ (pM) |
| wt | TCE-1 | 0.7 |
| R100A | TCE-3 | 3.4 |
| H101A | TCE-4 | 4.4 |
| N103A | TCE-6 | 168.5 |
| F104A | TCE-7 | 40.68 |
| S110A | TCE-13 | 89.76 |
| W112A | TCE-15 | 13.76 |

TABLE 38
TCE Cytotoxicity EC₅₀s (H292 Cells) - Mutated CDRL3

| Anti-CD3 scFv | | |
|---|---|---|
| CDRL3 | | H292 Cytotox |
| mutation | TCE Construct | EC₅₀ (pM) |
| wt | TCE-1 | 0.7 |
| W233A | TCE-19 | >1000 |
| W238A | TCE-24 | >1000 |
| F240A | TCE-26 | 19.96 |

TABLE 39
TCE Cytotoxicity EC₅₀s (H292 Cells)

| Anti-CD3 scFv | | |
|---|---|---|
| other | | H292 Cytotox |
| mutations | TCE Construct | EC₅₀ (pM) |
| wt | TCE-1 | 0.7 |
| other | TCE-27 | 0.5 |
| mutations | | |
| other | TCE-28 | 2.1 |
| mutations | | |

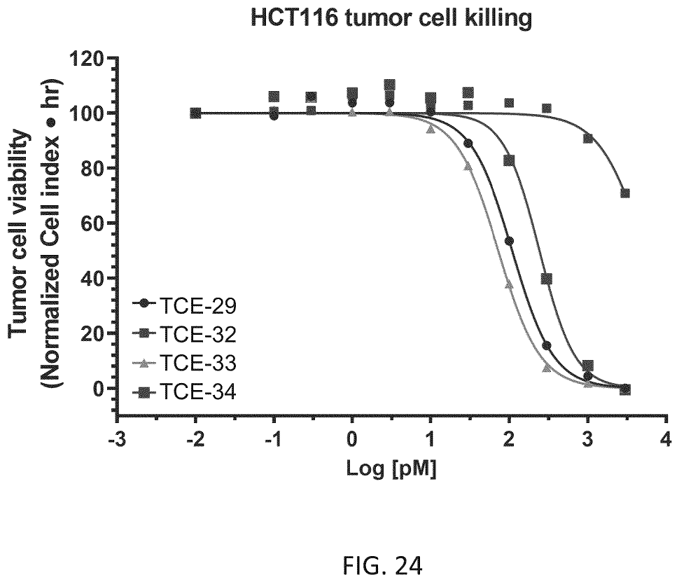

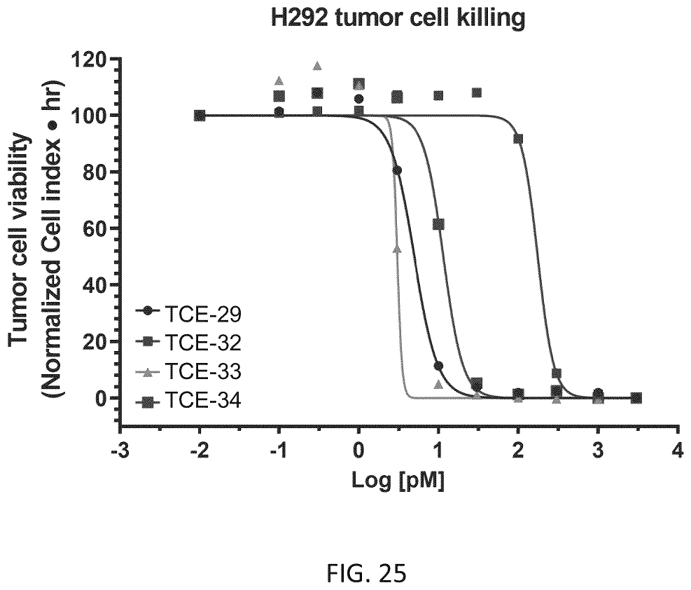

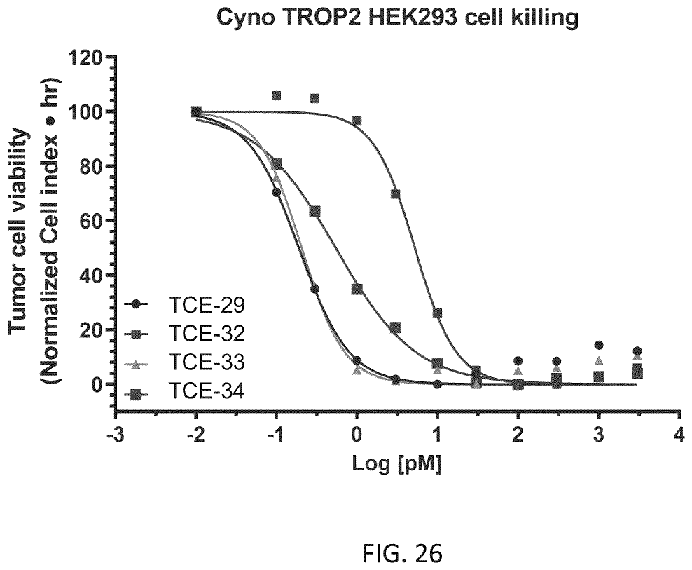

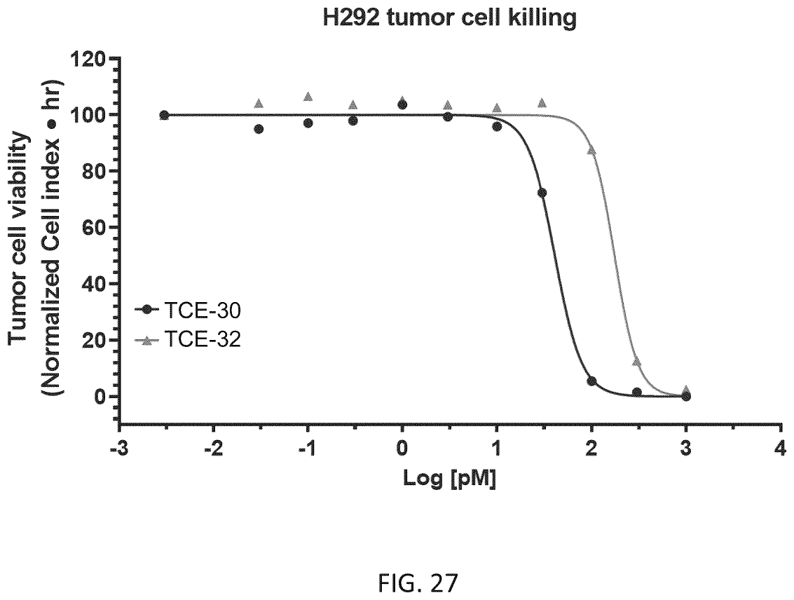

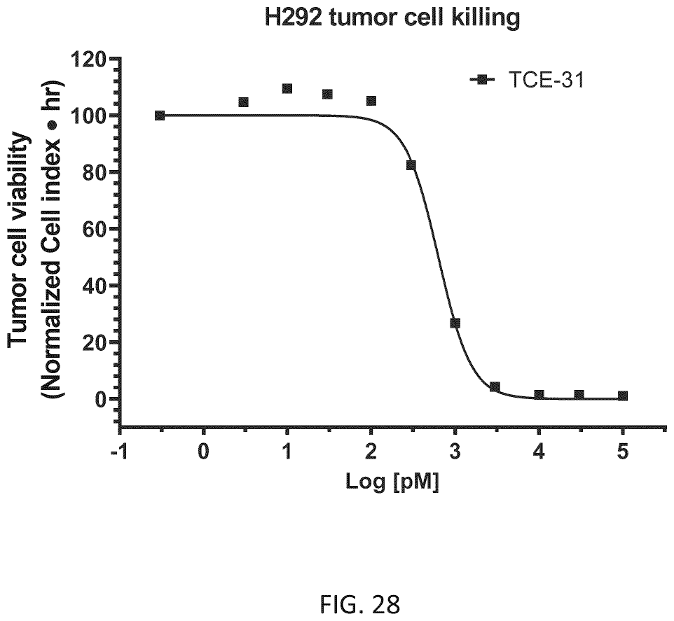

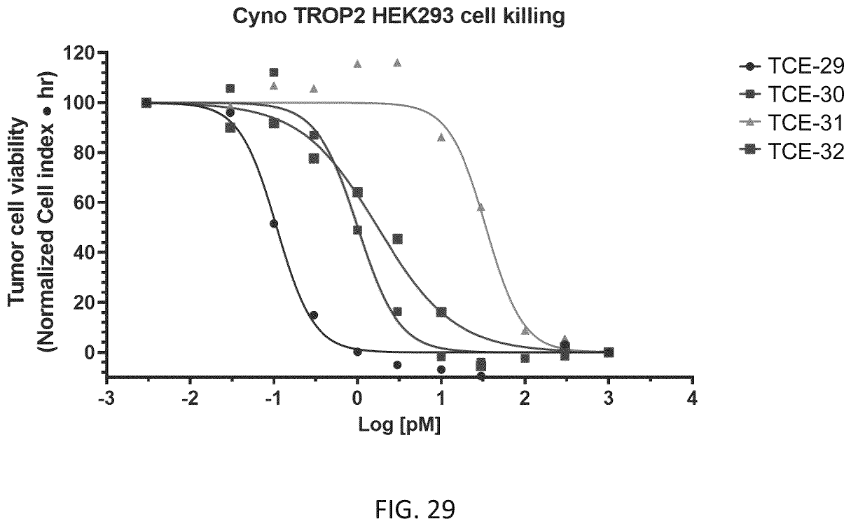

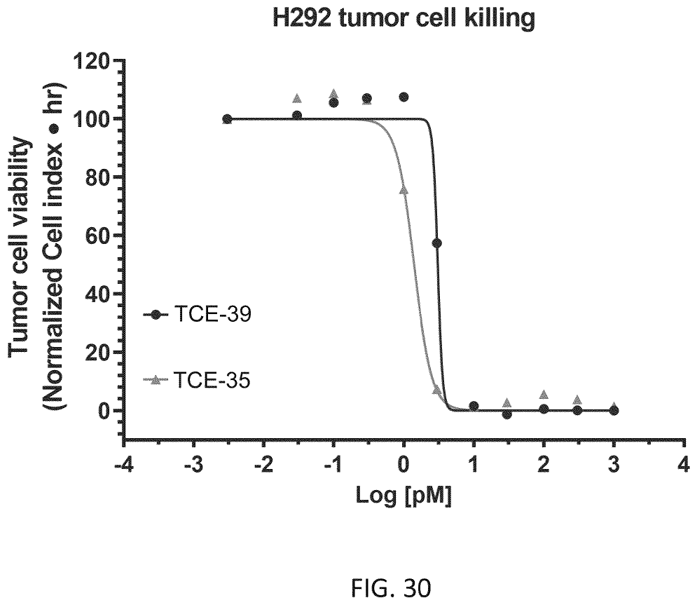

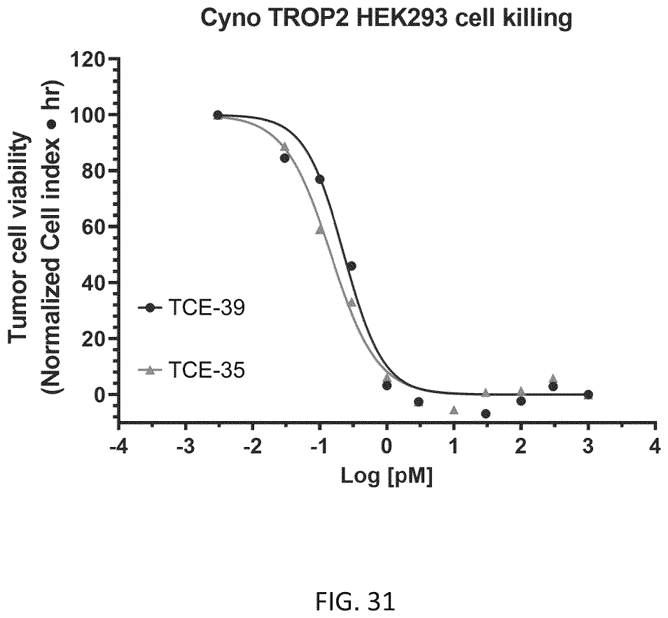

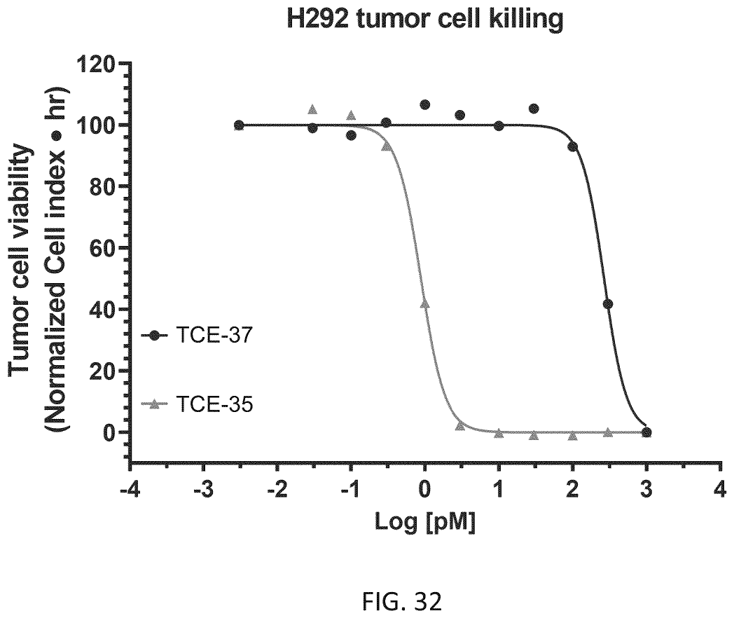

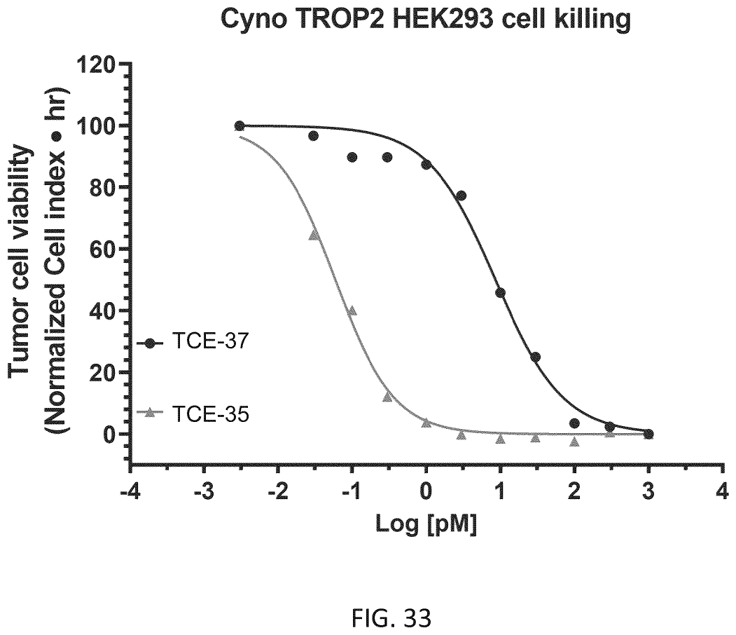

Data plots for tumor cell killing with TCE-29, TCE-32, TCE-33, and TCE-34 are shown in FIG. 24 (HCT116 cells), FIG. 25 (H292 cells), and FIG. 26 (cyno TROP2 HEK293 cells). Data plots for H292 tumor cell killing with TCE-30 and TCE-32 are shown in FIG. 27. A data plot for H292 tumor cell killing with TCE-31 is shown in FIG. 28. Data plots for cyno TROP2 HEK293 cell killing with TCE-29, TCE-30, TCE-31, and TCE-32 are shown in FIG. 29. Data plots for tumor cell killing with TCE-39 and TCE-35 are shown in FIG. 30 (H292 cells) and FIG. 31 (cyno TROP2 HEK293 cells). Data plots for tumor cell killing with TCE-37 and TCE-35 are shown in FIG. 32 (H292 cells) and FIG. 33 (cyno TROP2 HEK293 cells). The EC₅₀s for cytotoxicity of the different TCEs are provided in Tables 40-42.

TABLE 40
TCE Cytotoxicity EC₅₀s (HCT116 Cells)

| Anti-CD3 scFv | | HCT116 Cytotox |
|---|---|---|
| mutations | TCE Construct | EC₅₀ (pM) |
| wt | TCE-29 | 109 |
| F240A | TCE-32 | >3,000 |
| P41S, A49G, | TCE-33 | 73 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| N30Q, I109V, | TCE-34 | 243 |
| G172A, V231A | | |

TABLE 41
TCE Cytotoxicity EC₅₀s (H292 Cells)

| Anti-CD3 scFv | | H292 Cytotox |
|---|---|---|
| mutations | TCE Construct | EC₅₀ (pM) |
| wt | TCE-29 | 5 |
| F240A | TCE-32 | 175 |
| P41S, A49G, | TCE-33 | 3 |
| N87S, L150F, | | |
| T151S, G163R, | | |
| P175A, K181T, | | |
| T200V, A202D, | | |
| L208I, G211N, | | |
| L217I, S218T, | | |
| V220A, P222A, | | |
| E223D, A226S, | | |
| E227D | | |
| N30Q, I109V, | TCE-34 | 11 |
| G172A, V231A | | |
| H101A | TCE-30 | 41 |
| F104A | TCE-31 | 632 |
| N30Q, I109V, | TCE-39 | 3 |
| G172A, V231A | | |
| wt | TCE-35 | 1 |
| F104A | TCE-37 | 266 |

TABLE 42
TCE Cytotoxicity EC₅₀s (cynoTROP2+ HEK293 cells)

| Anti-CD3 scFv | | CynoTROP2+ HEK293 |
|---|---|---|
| mutations | TCE Construct | cells Cytotox EC₅₀ (pM) |
| wt | TCE-29 | 0.2 |
| F240A | TCE-32 | 5 |
| P41S, A49G, N87S, | TCE-33 | 0.2 |
| L150F, T151S, | | |
| G163R, P175A, | | |
| K181T, T200V, | | |
| A202D, L208I, | | |
| G211N, L217I, | | |
| S218T, V220A, | | |
| P222A, E223D, | | |
| A226S, E227D | | |
| N30Q, I109V, | TCE-34 | 0.5 |
| G172A, V231A | | |
| H101A | TCE-30 | 1 |
| F104A | TCE-31 | 34 |
| N30Q, I109V, | TCE-39 | 0.2 |
| G172A, V231A | | |
| wt | TCE-35 | 0.1 |
| F104A | TCE-37 | 9 |

### Example 5: Non-Human Primate (NHP) Studies of TROP2 TCEs

### NHP Toxicity Studies of TCE-29 and TCE-35

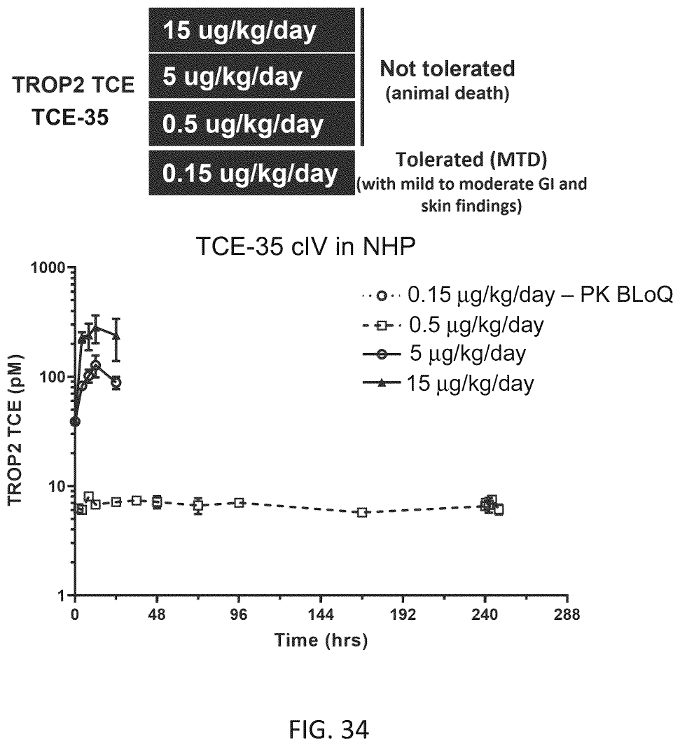

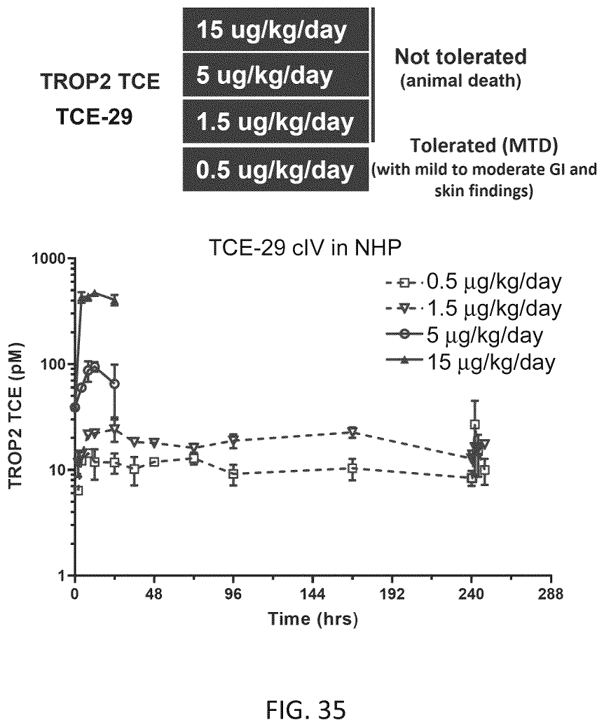

Pharmacokinetics and exploratory safety of TCE-29 (wt anti-CD3, anti-TROP2 F108A mutant) and TCE-35 (wt anti-CD3, anti-TROP2 D109A mutant) were evaluated in cynomolgus monkeys. Briefly, monkeys of approximately 3 kilograms (kg) bodyweight were administered TCEs as a continuous IV (cIV) infusion. Animals were observed for signs of adverse events. TCE-35 was dosed at 0.15 micrograms/kilogram/day (μg/kg/day), 0.5 μg/kg/day, 5 μg/kg/day, and 15 μg/kg/day. TCE-29 was dosed at 0.5 μg/kg/day, 1.5 μg/kg/day, 5 μg/kg/day, and 15 μg/kg/day. After dosing, blood was collected in K2 EDTA tubes at specific time points and processed to plasma. Plasma was frozen until analysis. Concentrations of TCEs in plasma were measured via a Meso Scale Discovery (MSD) based method relative to a reference standard diluted in control cynomolgus monkey plasma. The MSD based method significantly improved the lower limit of quantification of the TCEs in plasma. Pharmacokinetic profiles and toxicity results are shown in FIG. 34 (TCE-35) and FIG. 35 (TCE-29). Both TCE-35 and TCE-29 showed a dose proportional exposure increase in general. As shown in the figures, the maximum tolerated doses (MTD) for TCE-35 and TCE-29 were 0.15 μg/kg/day and 0.5 μg/kg/day, respectively. Mild to moderate gastrointestinal (GI) and skin findings were observed at these doses.

Cytokine Induction in NHPs with TCE-29 and TCE-35

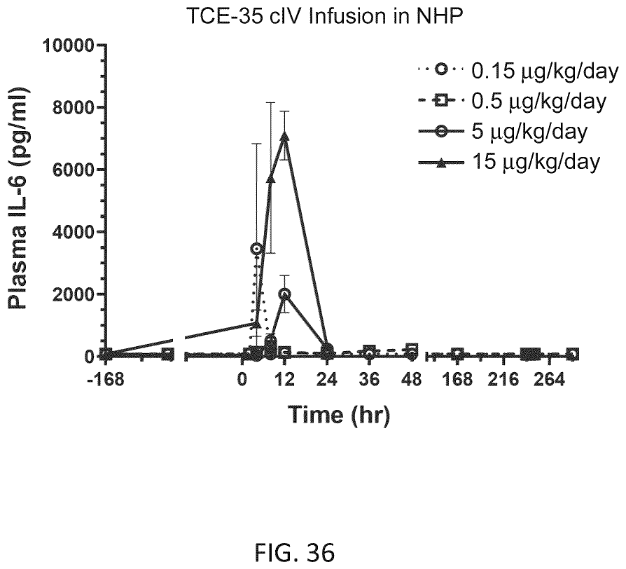

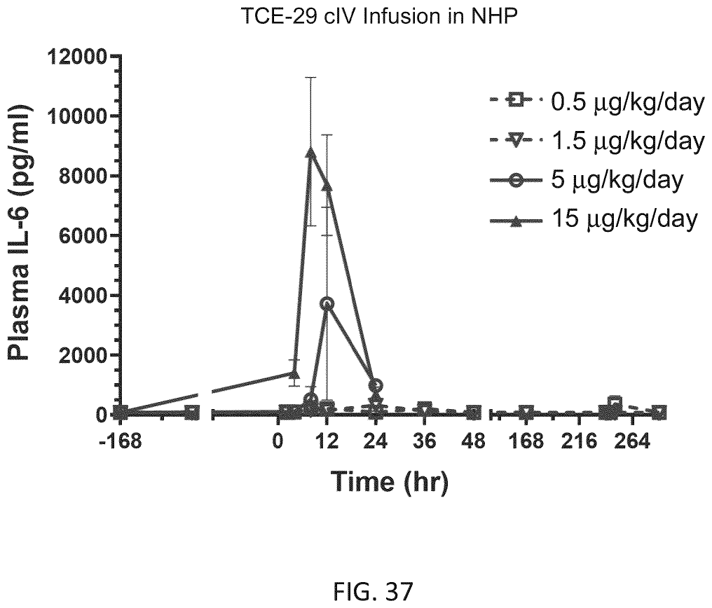

Cytokine release after TCE-29 and TCE-35 administration by cIV was evaluated in cynomolgus monkeys. Briefly, cynomolgus monkeys of approximately 3 kg bodyweight were implanted with an infusion pump subcutaneously. Two weeks later the pump was filled with TCE dosing solution and administered via constant infusion. After dosing started, blood was collected in K2 EDTA tubes at specific timepoints and processed to plasma. Plasma samples were analyzed for cytokines using a non-human primate cytometric Th1/Th2 bead array kid from BD biosciences following the manufacturer's instructions. Interleukin 6 (IL-6) levels in plasma were calculated relative to reference standards provided with the bead array kit. FIGS. 36-37 show IL-6 release in cynomolgus monkey after continuous infusion of TCE-35 (FIG. 36) and TCE-29 (FIG. 37) at different doses. IL-6 levels (in picograms/milliliter (pg/mL)) following cIV infusion for 10 days at different doses are provided in Table 43 for TCE-35 and Table 44 for TCE-29.

TABLE 43
NHP Continuous IV Infusion of TCE-35 for 10 Days

| 0.15 μg/kg/day | 0.5 μg/kg/day | 5 μg/kg/day | 15 μg/kg/day |
|---|---|---|---|
| 3458 pg/mL | 298 pg/mL | 2006 pg/mL | 7098 pg/mL |
| IL-6 | IL-6 | IL-6 | IL-6 |

TABLE 44
NHP Continuous IV Infusion of TCE-29 for 10 Days

| 0.5 μg/kg/day | 1.5 μg/kg/day | 5 μg/kg/day | 15 μg/kg/day |
|---|---|---|---|
| 356 pg/mL | 324 pg/mL | 3719 pg/mL | 8811 pg/mL |
| IL-6 | IL-6 | IL-6 | IL-6 |

### Clinical Chemistry of TCE-29 and TCE-35 in NHP

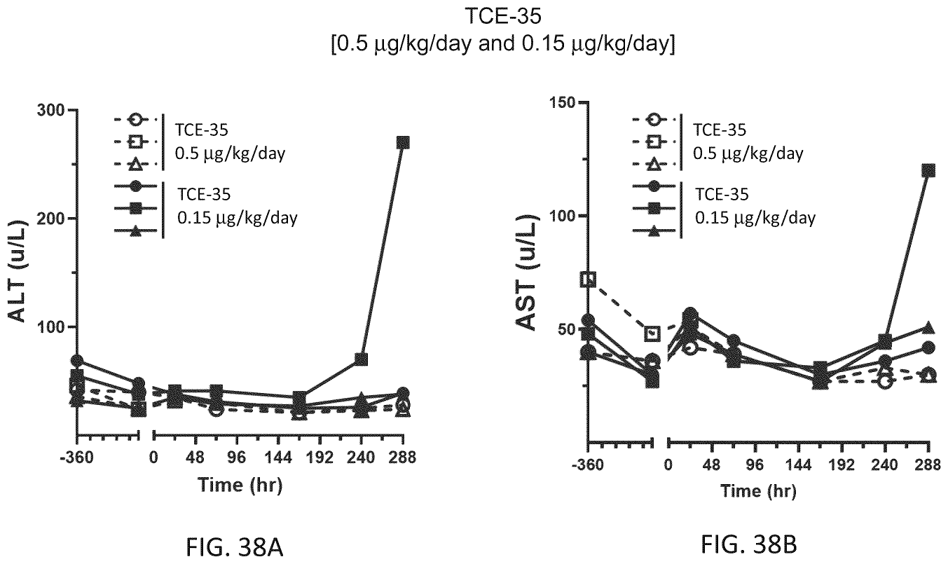

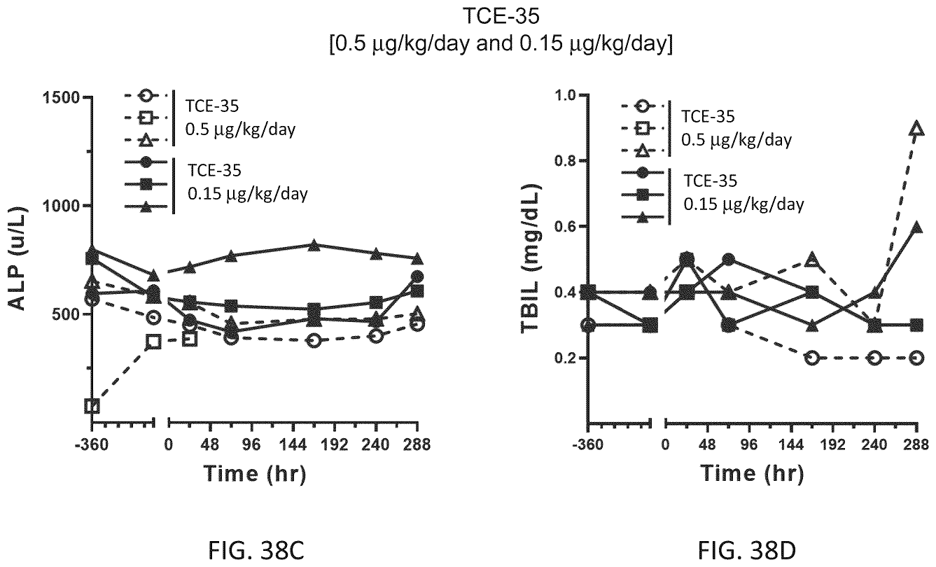

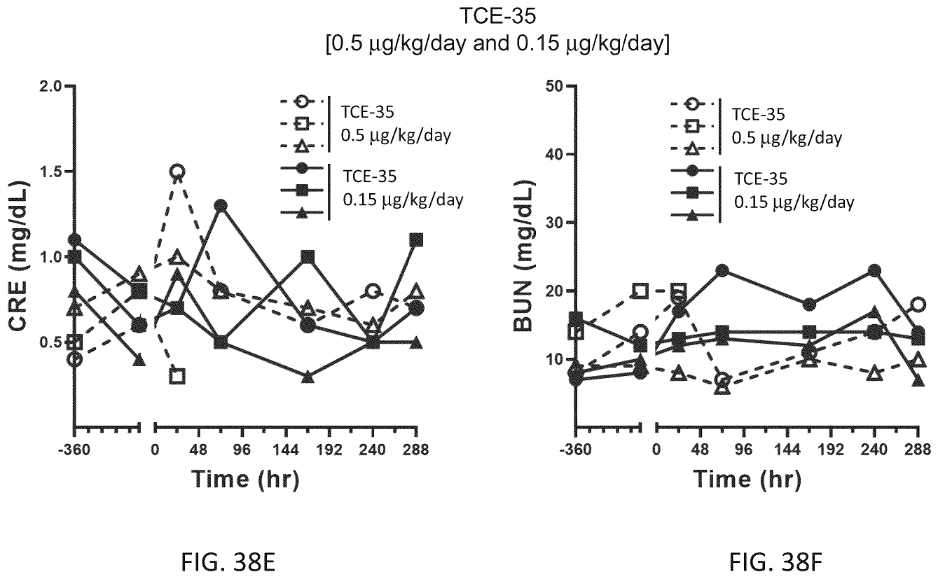

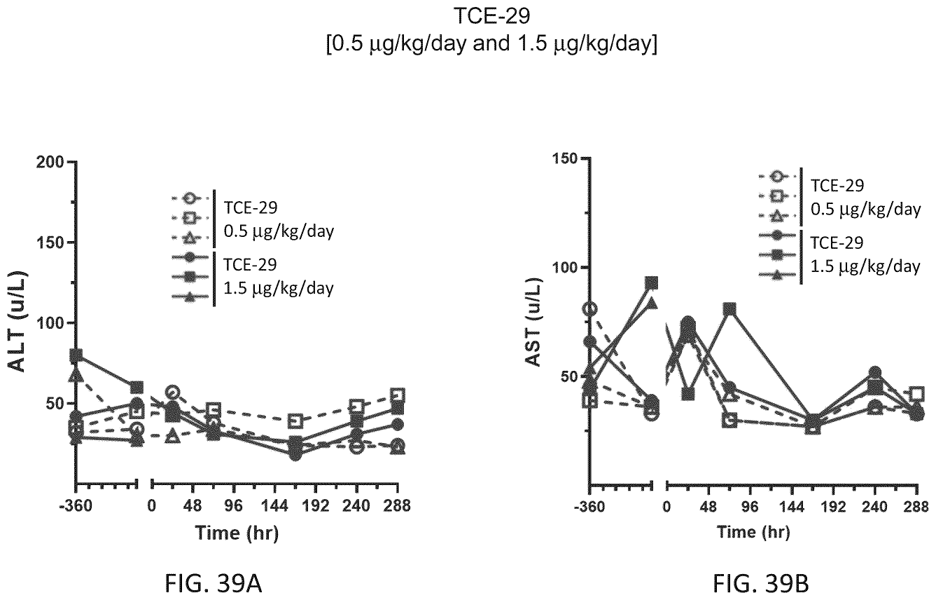

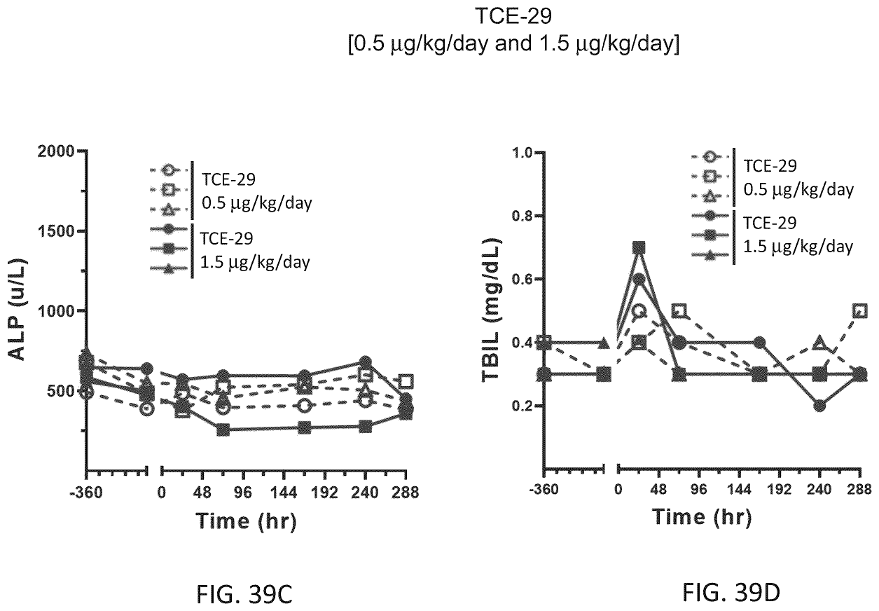

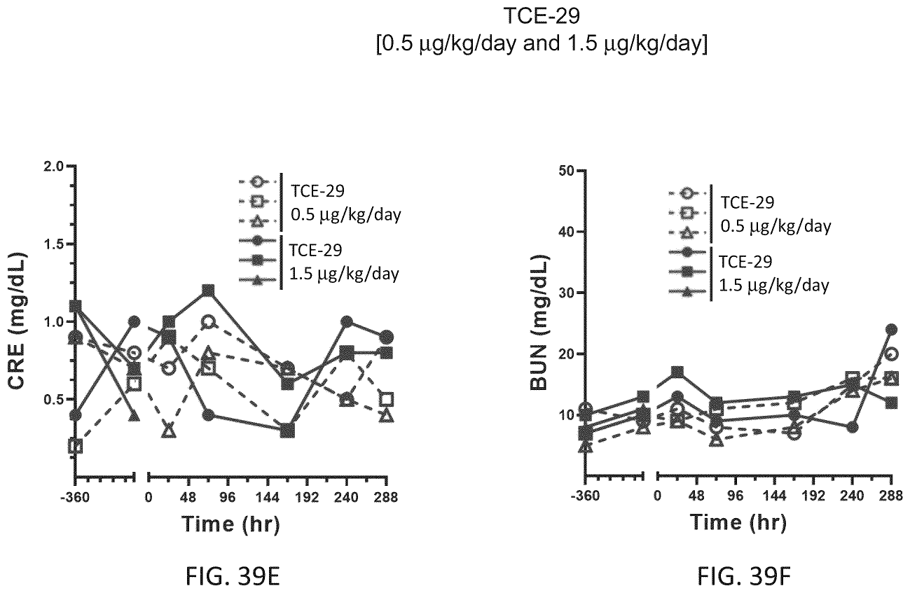

Clinical chemistry after TCE-29 and TCE-35 administration by cIV in cynomolgus monkeys was measured through standard panel analyses. Briefly, cynomolgus monkeys of approximately 3 kg bodyweight were administered TCEs by cIV and observed daily for signs of adverse events. After dosing, blood was collected in K2 EDTA tubes at specific timepoints and processed to plasma. Clinical chemistry parameters were run on freshly prepared plasma samples, including alanine aminotransferase (ALT), aspartate transaminase (AST), alkaline phosphatase (ALP), bilirubin (TBIL), creatinine (CRE), and blood urea nitrogen (BUN) as signs of liver and kidney related toxicity. FIGS. 38-39 show plots of ALT, AST, ALP, TBIL, CRE, and BUN levels in cynomolgus monkeys after administration of TCE-35 (FIGS. 38A-38F) and TCE-29 (FIG. 39A-39F).

### NHP Toxicity Studies of TCE-31, TCE-32, and TCE-34

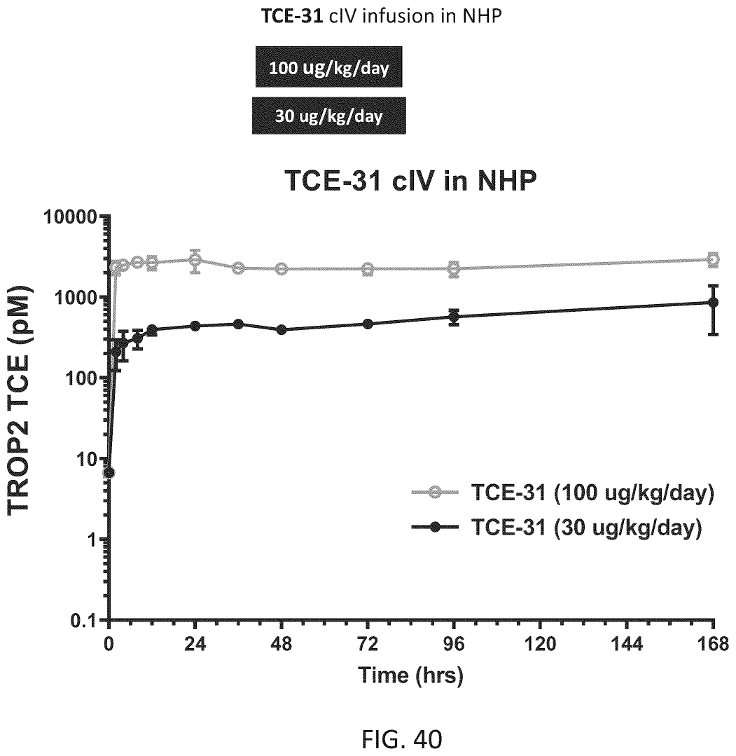

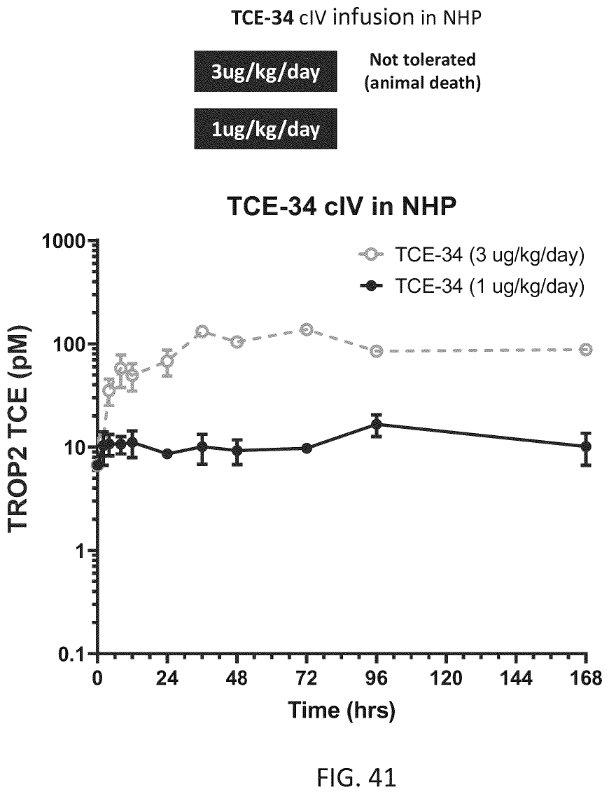

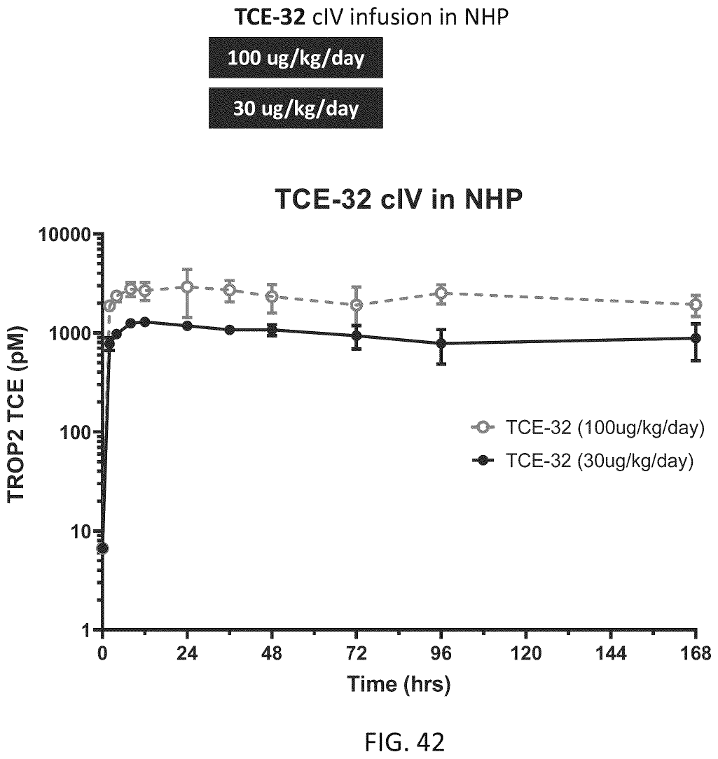

Pharmacokinetics and exploratory safety of TCE-31, TCE-32, and TCE-34 were evaluated as described above. Pharmacokinetic profiles and toxicity results are shown in FIG. 40 (TCE-31), FIG. 41 (TCE-34), and FIG. 42 (TCE-32). Maximum tolerated doses were 30 μg/kg/day for TCE-31, 1 μg/kg/day for TCE-34, and 30 μg/kg/day for TCE-32.

Cytokine Induction in NHPs with TCE-31, TCE-32, and TCE34

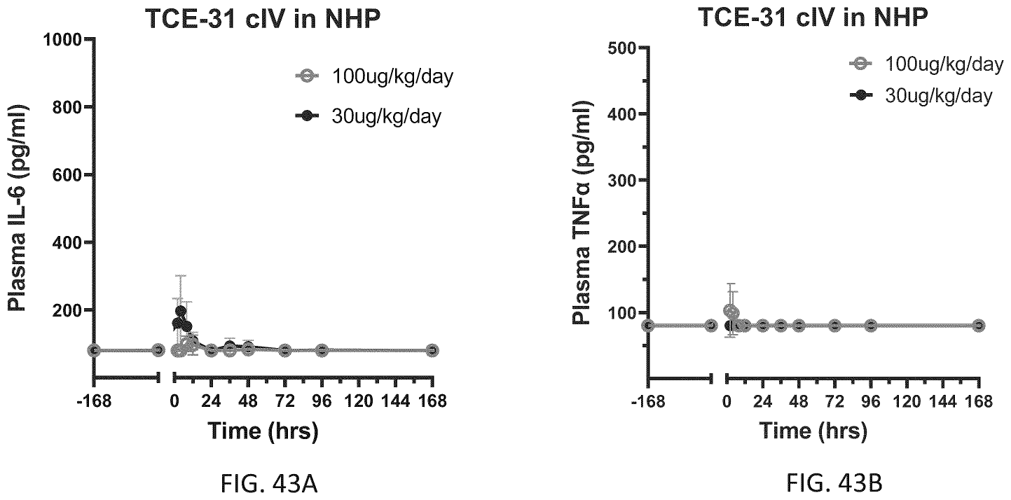

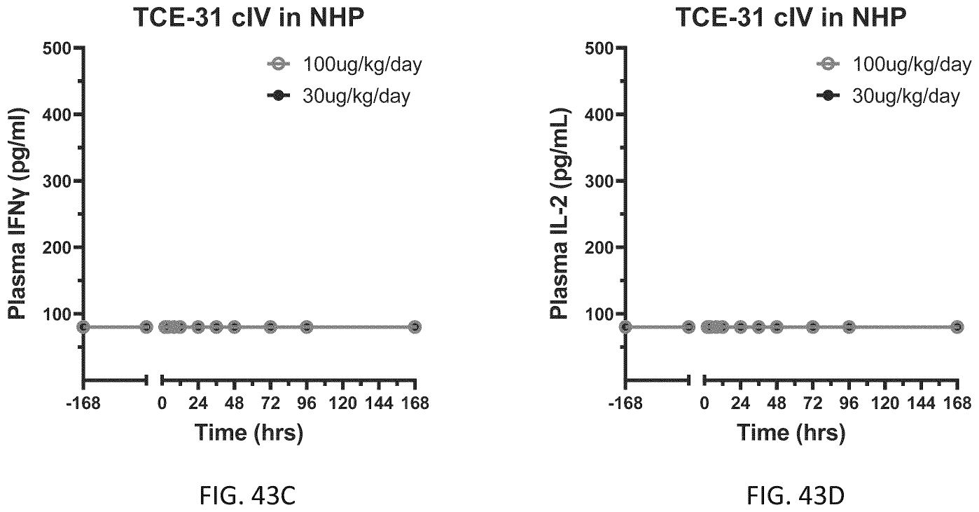

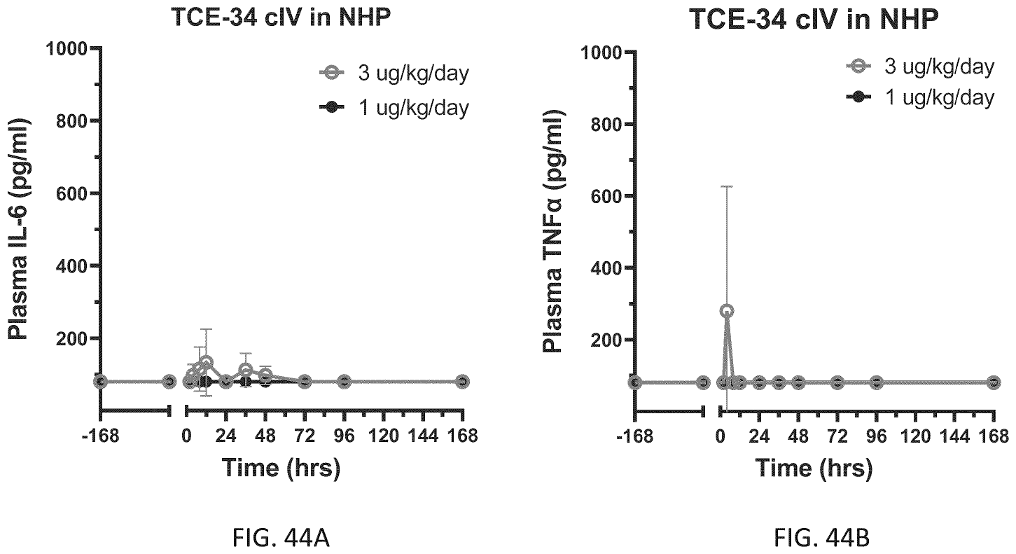

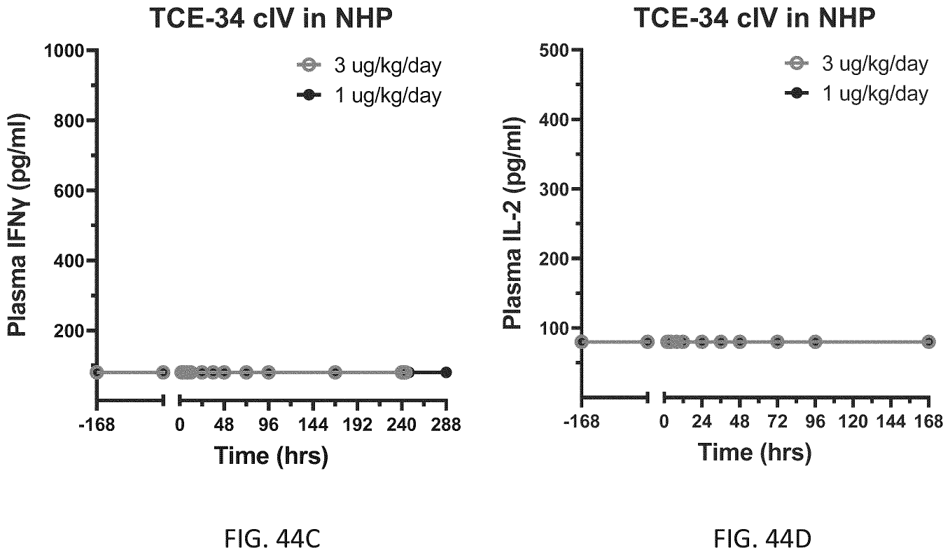

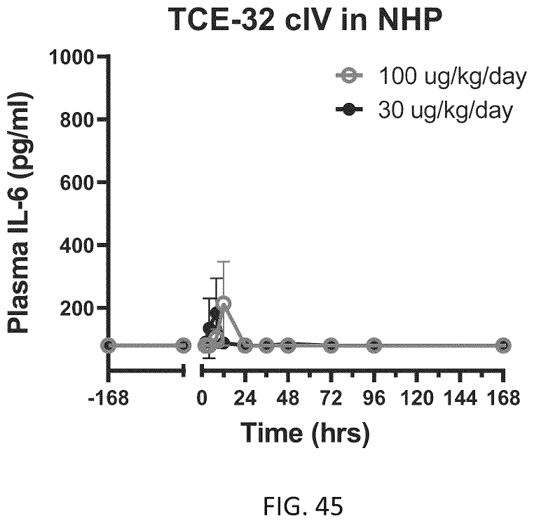

Cytokine release after TCE-31, TCE-32, and TCE-34 administration by cIV was evaluated in cynomolgus monkeys as described above. FIGS. 43A-43D show release of IL-6, tumor necrosis factor alpha (TNFα), interferon gamma (IFNγ), and interleukin-2 (IL-2) in cynomolgus monkey after continuous infusion of TCE-31. FIGS. 44A-44D show release of IL-6, TNFα, IFNγ, and IL-2 in cynomolgus monkey after continuous infusion of TCE-34. FIG. 45 shows release of IL-6 in cynomolgus monkey after continuous infusion of TCE-32. IL-6 levels in pg/mL following cIV infusion for 10 days at different doses are provided in Table 45 for TCE-31, Table 46 for TCE-32, and Table 47 for TCE-34. Notably, TCE-31 (F104A anti-CD3 mutant) and TCE-32 (F240A anti-CD3 mutant) resulted in reduced IL-6 levels at the maximum tolerated doses after 10 days compared to TCE-29 which has the wild-type anti-CD3 sequence (compare Tables 44-46), suggesting a potential advantage for the anti-CD3 alanine mutants related to cytokine release syndrome.

TABLE 45
NHP Continuous IV Infusion of TCE-31 for 10 Days

| 30 μg/kg/day | 100 μg/kg/day |
|---|---|
| 197 pg/mL IL-6 | 100 pg/mL IL-6 |

TABLE 46
NHP Continuous IV Infusion of TCE-32 for 10 Days

| 30 μg/kg/day | 100 μg/kg/day |
|---|---|
| 183 pg/mL IL-6 | 213 pg/mL IL-6 |

TABLE 47
NHP Continuous IV Infusion of TCE-34 for 10 Days

| 1 μg/kg/day | 3 μg/kg/day |
|---|---|
| IL-6, below limit | 133 pg/mL IL-6 |
| of quantitation | |

### Clinical Chemistry of TCE-31, TCE-32, and TCE-34

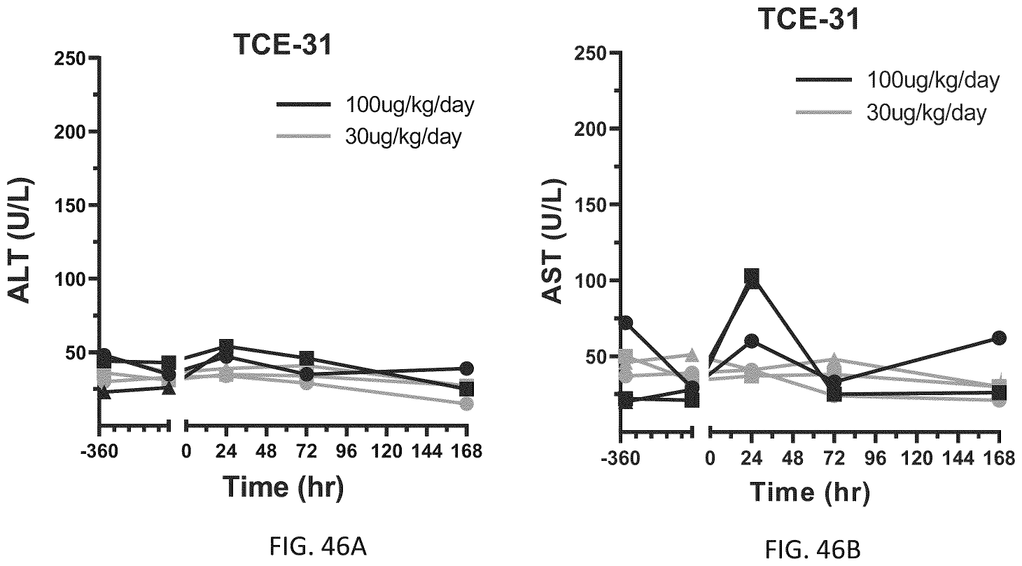

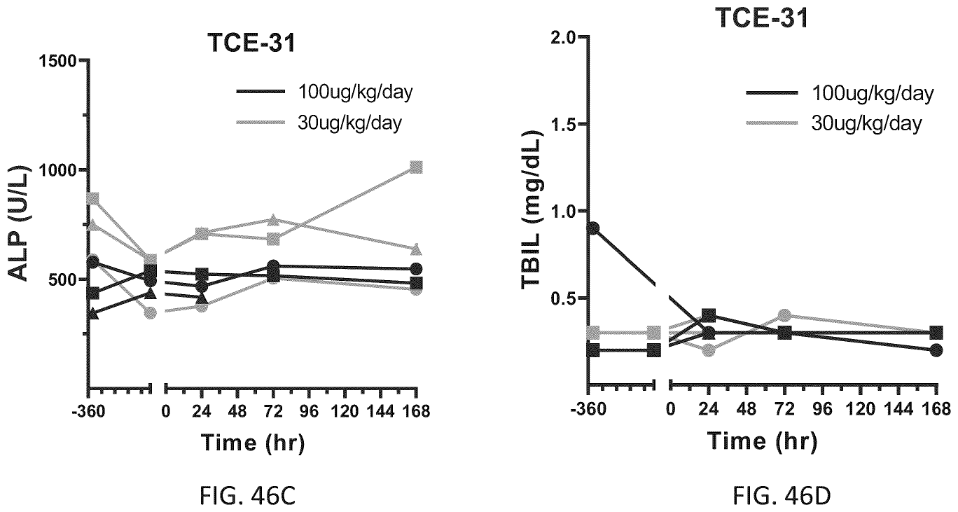

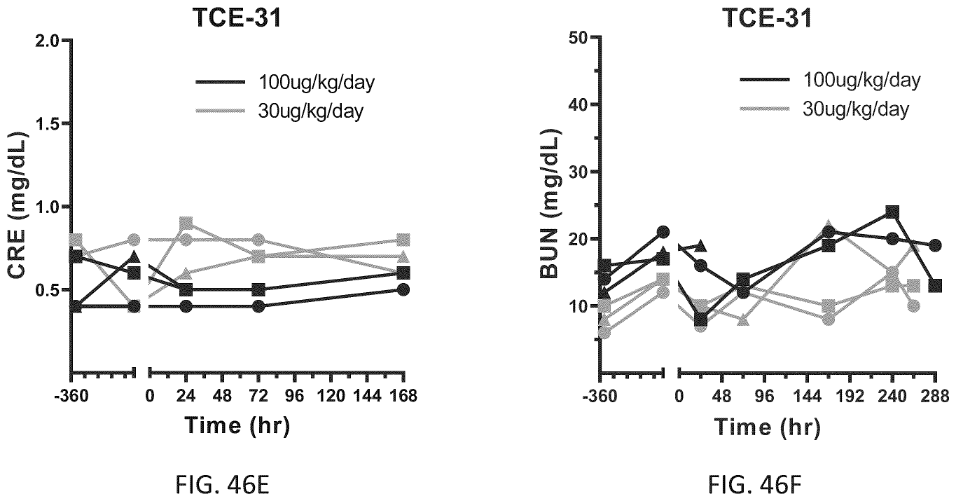

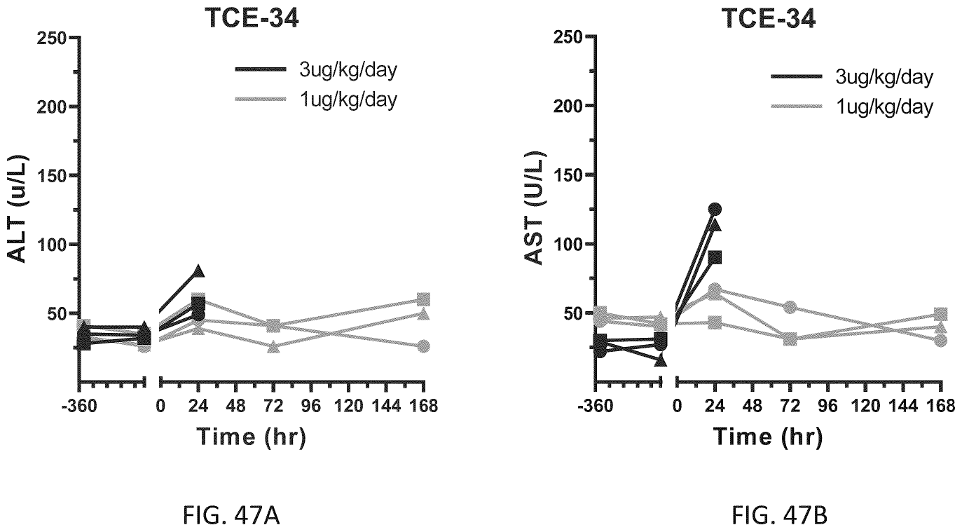

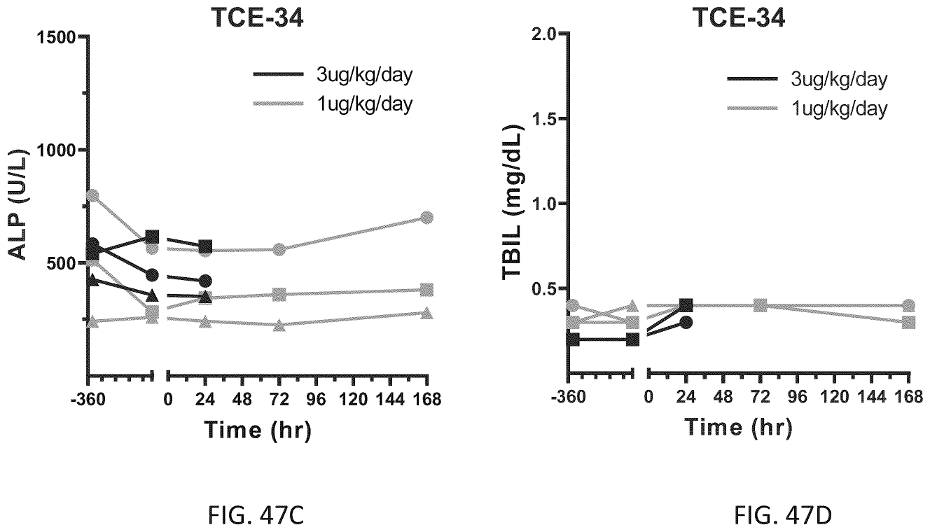

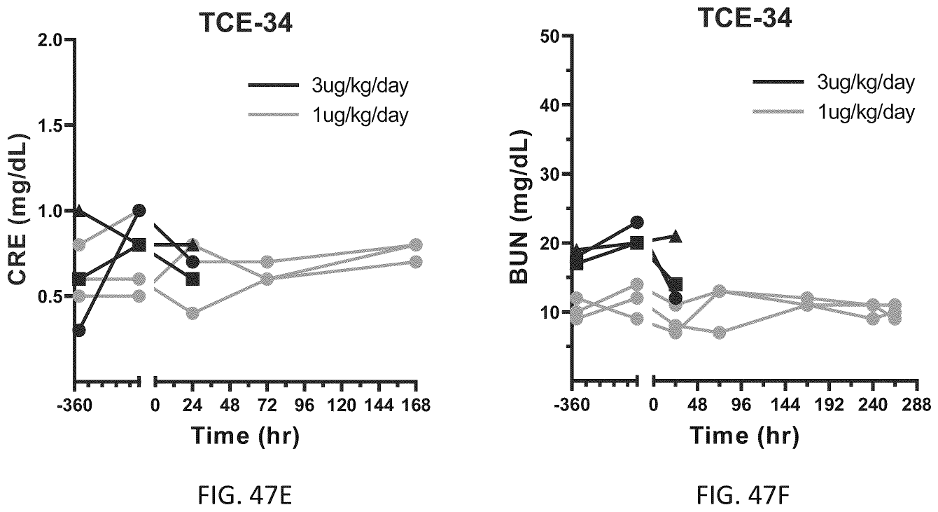

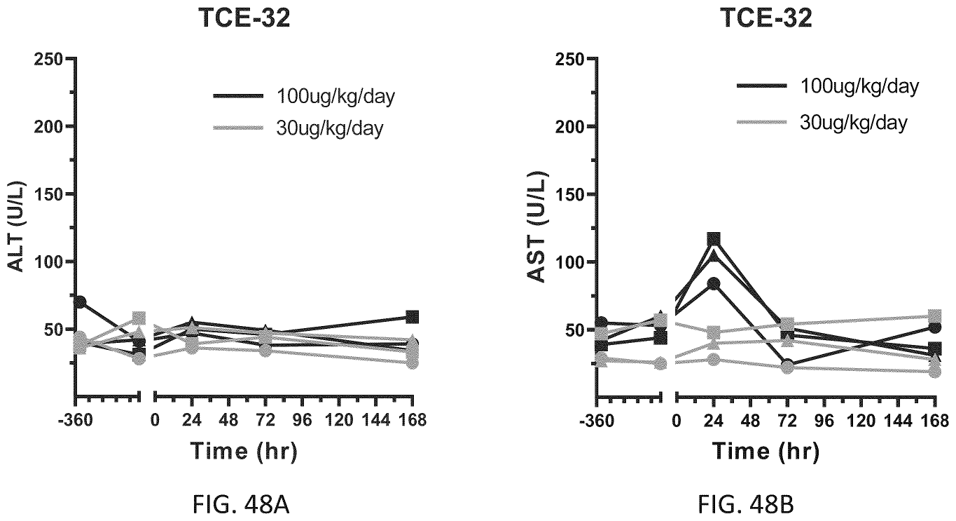

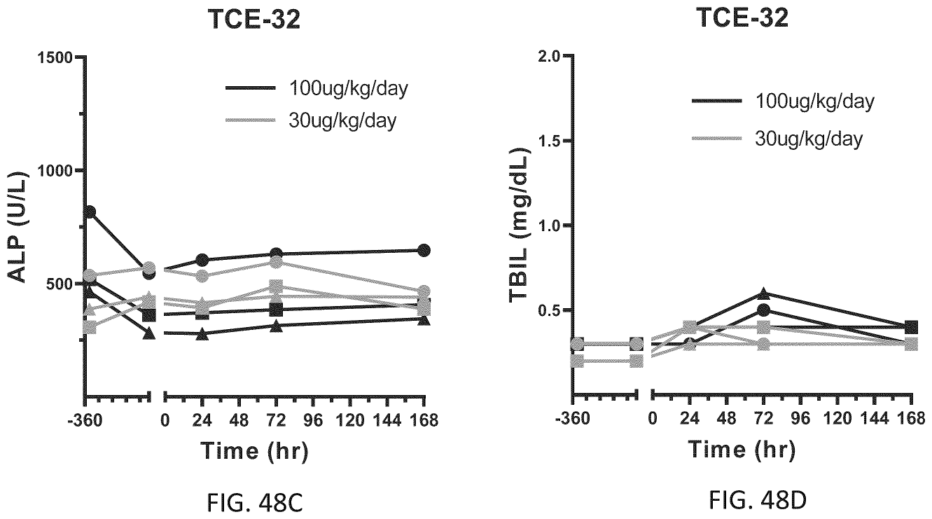

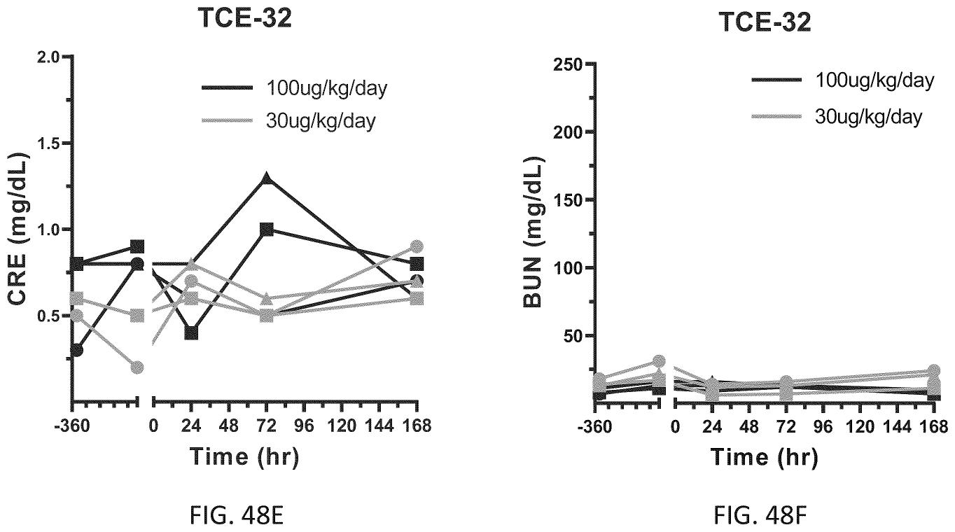

Clinical chemistry after TCE-31, TCE-32, and TCE-34 administration by cIV in cynomolgus monkeys was measured through standard panel analyses as described above. FIGS. 46-48 show plots of ALT, AST, ALP, TBIL, CRE, and BUN levels in cynomolgus monkeys after administration of TCE-31 (FIGS. 46A-46F), TCE-34 (FIGS. 47A-47F), and TCE-32 (FIGS. 48A-48F).

### Example 6: Binding of TROP2 TCEs to TROP2 and CD3

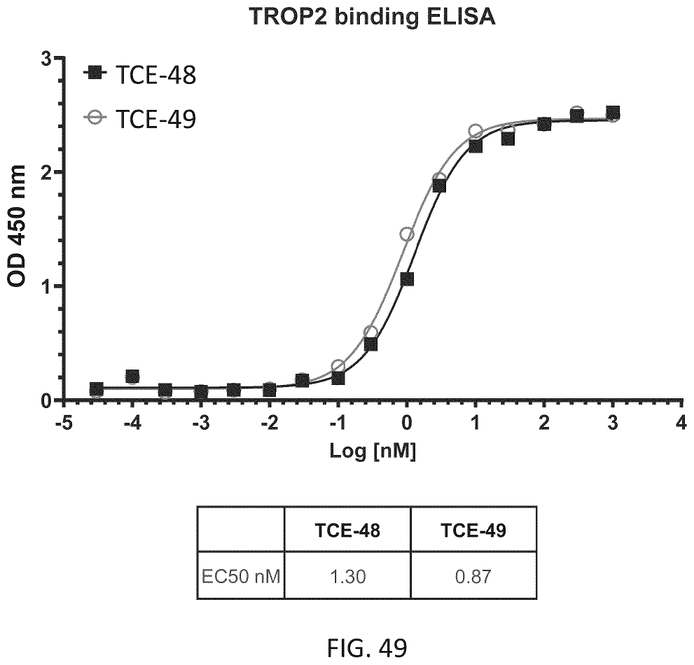

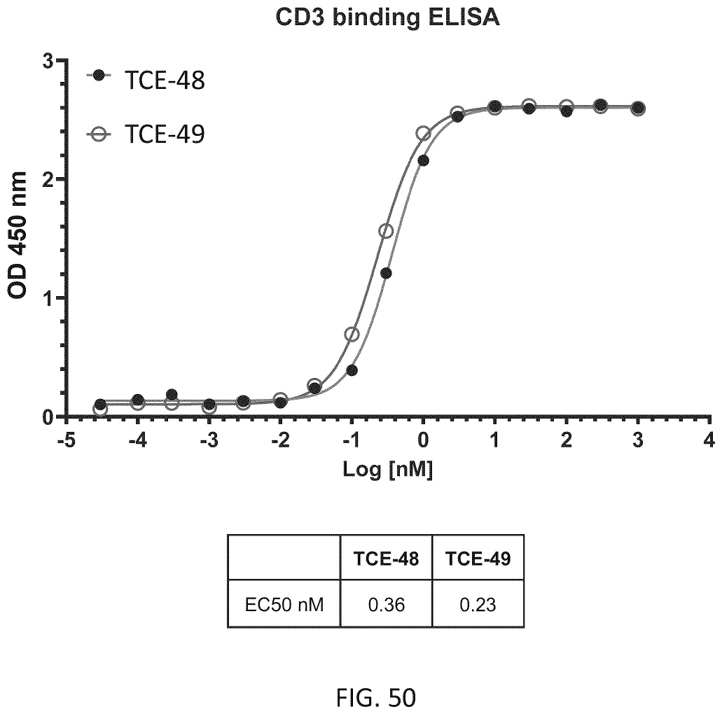

Equilibrium binding of TCE-48 and TCE-49 to TROP2 and CD3 was evaluated using an ELISA format. Briefly, biotinylated CD3 or biotinylated TROP2 was captured on neutravidin coated plates. The TCE constructs diluted in buffer were then added to the antigen coated plates. Binding was detected using a standard horse radish peroxidase secondary antibody. The concentration of the TCE required to achieve 50% maximal signal (EC₅₀) was calculated using Graphpad Prism software. Binding curves and EC₅₀s for TROP2 binding by TCE-48 and TCE-49 are shown in FIG. 49. Binding curves and EC₅₀s for CD3 binding by TCE-48 and TCE-49 are shown in FIG. 50.

### Example 7: Tumor Cell Killing with TROP2 TCEs

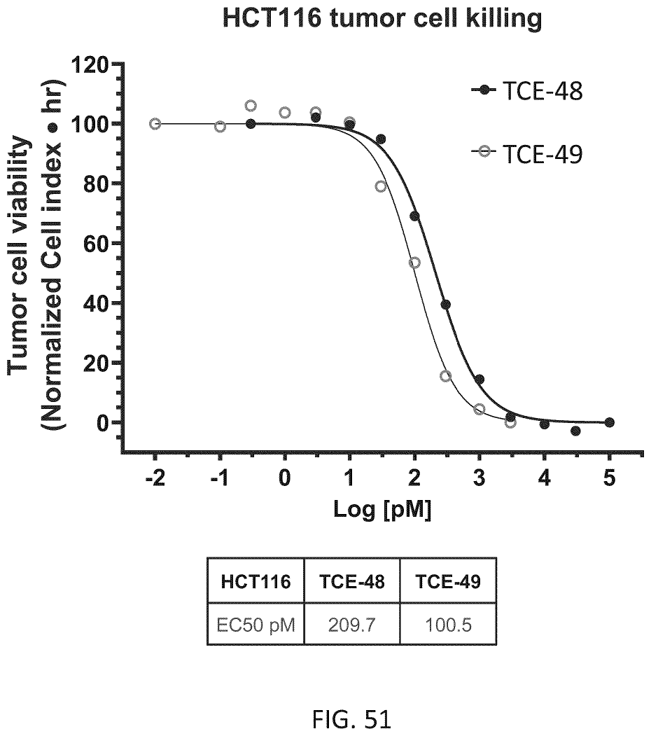

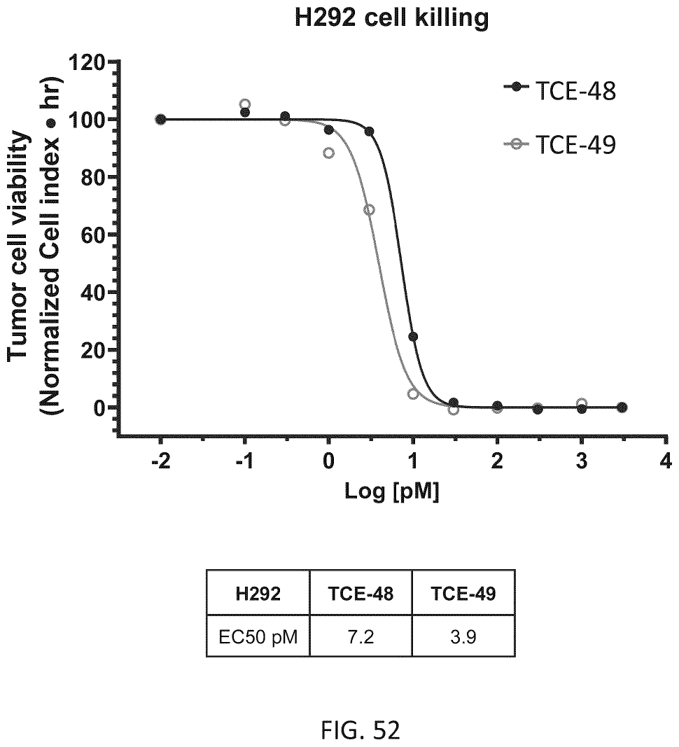

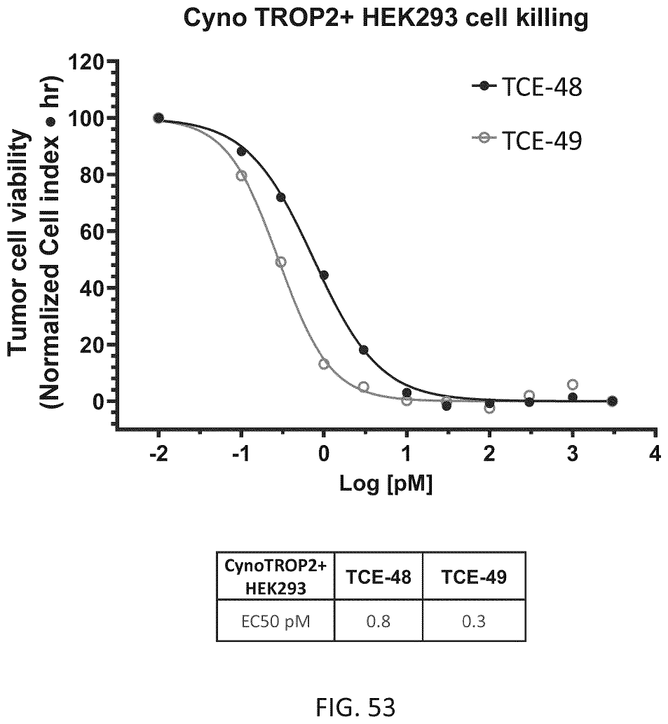

TROP2 TCEs were evaluated in a functional in vitro tumor cell killing assay using TROP2 positive HCT116, H292, or HEK293 tumor cell lines. Recombinant HEK293 cell lines were engineered to express cynomolgus monkey TROP2 on their surface (Cyno TROP2+HEK293). Tumor cell killing was measured and EC₅₀s were calculated as described above in Example 4. FIG. 51 shows data plots and EC₅₀s for HCT116 tumor cell killing with TCE-48 and TCE-49. FIG. 52 shows data plots and EC₅₀s for H292 tumor cell killing with TCE-48 and TCE-49. FIG. 53 shows data plots and EC₅₀s for Cyno TROP2+HEK293 tumor cell killing with TCE-48 and TCE-49.

### Example 8: Binding of EGFR TCEs to EGFR and CD3

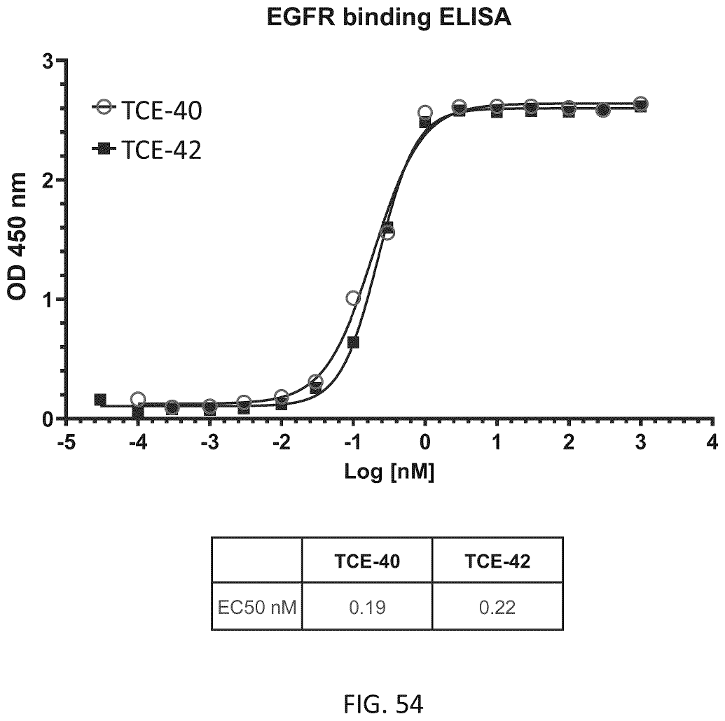

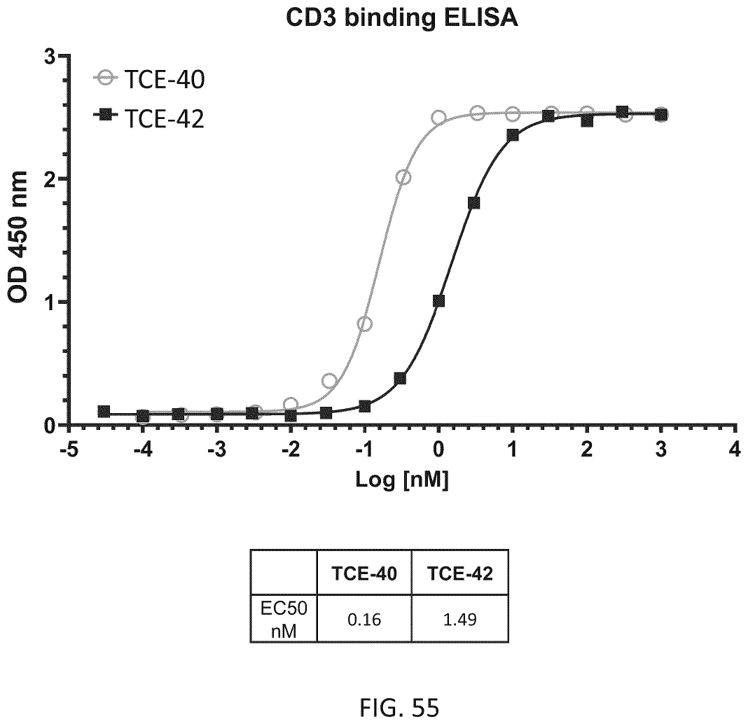

Binding of EGFR TCEs to EGFR and CD3 was evaluated using ELISA. Biotinylated EGFR or biotinylated CD3 was captured on neutravidin coated plates. TCEs diluted in buffer were added to the antigen coated plates. Binding was detected using a standard horseradish peroxidase conjugated secondary antibody. The concentration of the TCE required to achieve 50% maximal signal (EC₅₀) was calculated using Graphpad Prism software. Binding curves and EC₅₀s for EGFR binding by TCE-40 and TCE-42 are shown in FIG. 54. Binding curves and EC₅₀s for CD3 binding by TCE-40 and TCE-42 are shown in FIG. 55.

### Example 9: Tumor Cell Killing with EGFR TCEs

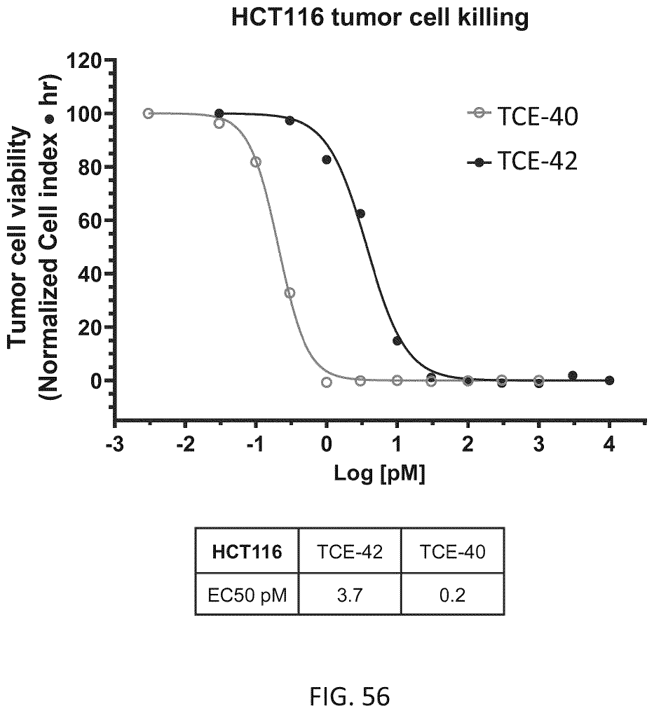

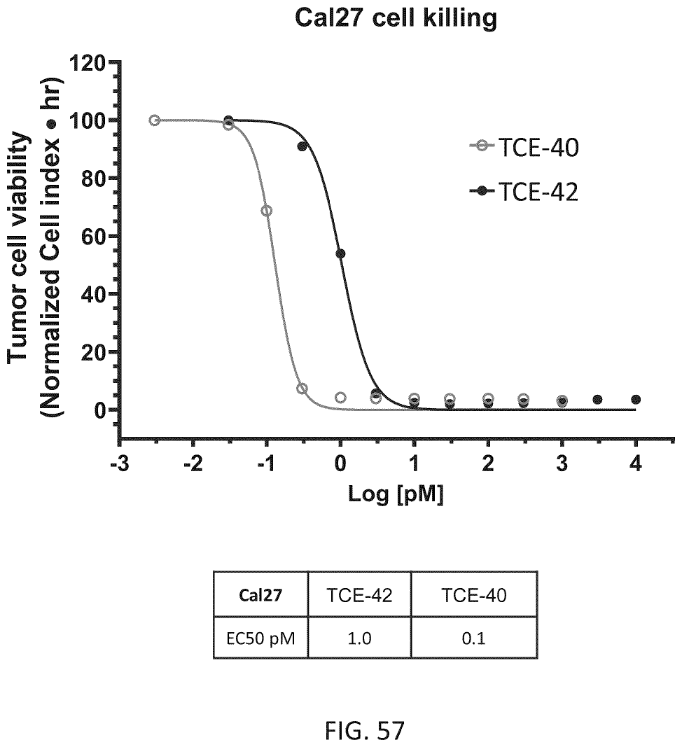

EGFR TCEs were evaluated in a functional in vitro tumor cell killing assay using EGFR positive tumor cell lines HCT116 and Cal27. Tumor cell killing was measured using xCelligence real time analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day TCE constructs titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of TCE where the concentration required to reduce the tumor growth 50% (EC₅₀) was calculated using Graphpad Prism software. FIG. 56 shows data plots and EC₅₀s for HCT116 tumor cell killing with TCE-40 and TCE-42. FIG. 57 shows data plots and EC₅₀s for Cal27 tumor cell killing with TCE-40 and TCE-42.

### Example 10: Binding of PSMA TCEs to PSMA and CD3

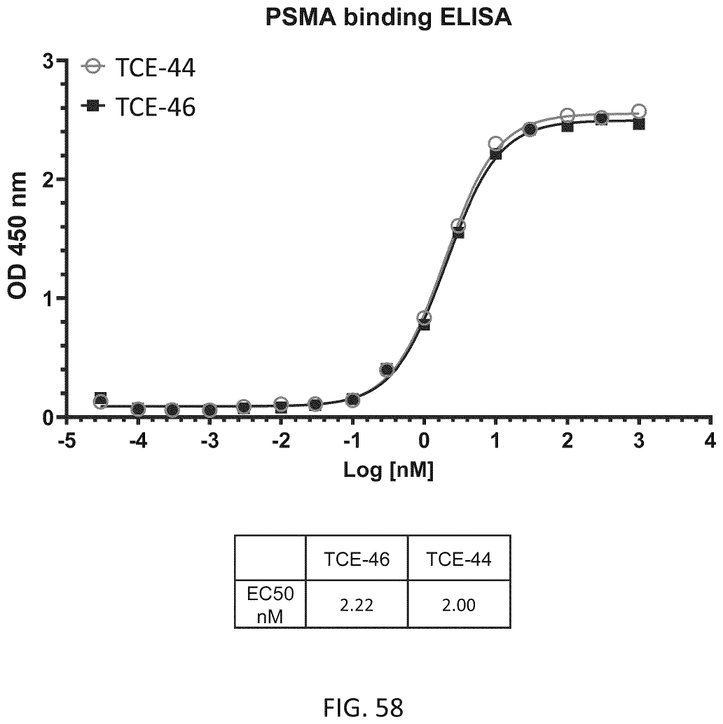

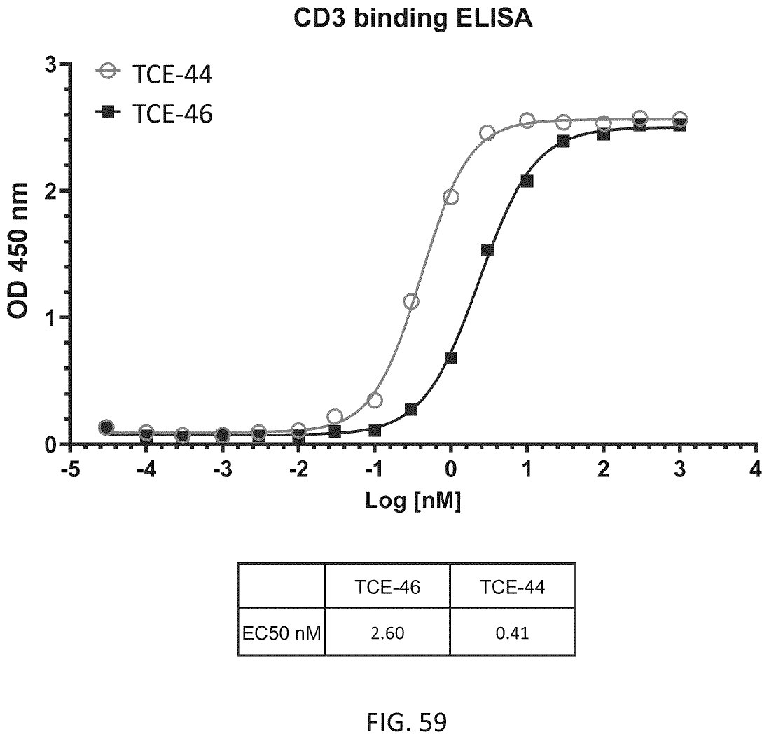

PSMA TCEs were evaluated for their ability to bind PSMA and CD3 in an ELISA format. Biotinylated PSMA or biotinylated CD3 was captured on neutravidin coated lates. TCEs diluted in buffer were then added to the antigen coated plates. Binding was detected using a standard horse radish peroxidase conjugated secondary antibody. The concentration of TCE required to achieve 50% maximal signal (EC₅₀) was calculated using Graphpad Prism software. Binding curves and EC₅₀s for PSMA binding by TCE-44 and TCE-46 are shown in FIG. 58. Binding curves and EC₅₀s for CD3 binding by TCE-44 and TCE-46 are shown in FIG. 59.

### Example 11: Tumor Cell Killing with PSMA TCEs

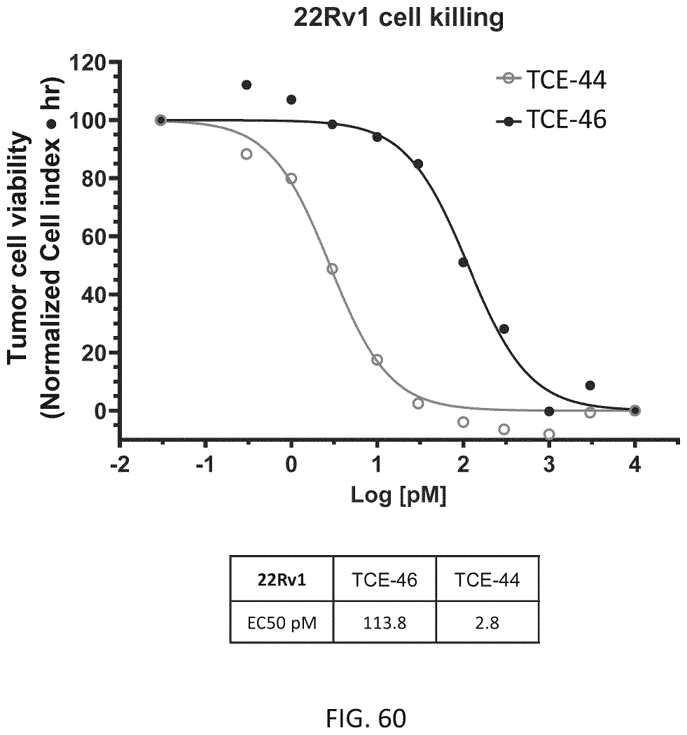

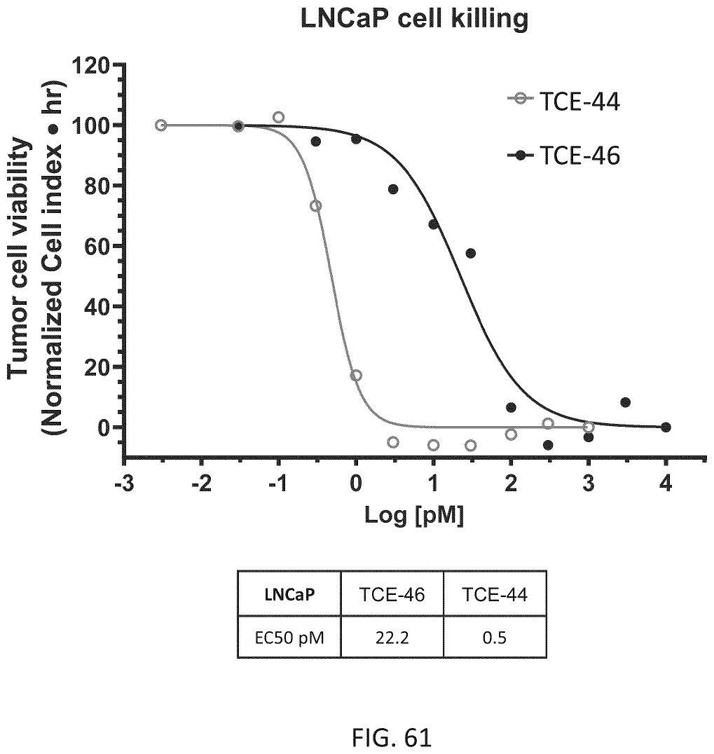

PSMA TCEs were evaluated in a functional in vitro tumor cell killing assay using the PSMA positive tumor cell lines 22Rv1 and LNCaP. Tumor cell killing was measured using xCelligence real time analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight. The following day TCEs titrated in human serum supplemented medium along with 30,000 CD8+ T cells were added to the wells. Cell index measurements were taken every 10 minutes for an additional 96 hours. The cell index times number of hours (tumor cell growth kinetics) was then plotted versus concentration of TCE where the concentration required to reduce the tumor growth 50% (EC₅₀) was calculated using Graphpad Prism software. FIG. 60 shows data plots and EC₅₀s for 22Rv1 tumor cell killing with TCE-44 and TCE-46. FIG. 61 shows data plots and EC₅₀s for LNCaP tumor cell killing with TCE-44 and TCE-46.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A recombinant antibody or antigen binding fragment thereof that comprises a CD3 binding domain that comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 1;
wherein CDR2-L comprises the amino acid sequence of SEQ ID NO: 2 (GTK);
wherein CDR3-L comprises an amino acid sequence of X₁-X₂-W-X₃-X₄-X₅-X₆-W-X₇-X₈;
wherein X₁ is V, G, P, L, I, M, S, T, or A;
X₂ is L, G, P, V, I, M, S, T, or A;
X₃ is Y, F, W, V, L, I, G, or A;
X₄ is S, G, T, M, N, Q, H, or A;
X₅ is N, Q, S, T, D, E, H, K, R, or A;
X₆ is R, S, T, Q, D, E, H, K, N, or A;
X₇ is V, G, P, L, I, M, S, T, or A; and
X₈ is F, Y, W, V, L, I, G, or A;
wherein CDR1-H comprises the amino acid sequence of SEQ ID NO: 15;
wherein CDR2-H comprises the amino acid sequence of SEQ ID NO: 16;
wherein CDR3-H comprises an amino acid sequence of X₉-X₁₀-X₁₁-X₁₂-N-X₁₃-X₁₄-X₁₅-X₁₆-X₁₇-X₁₈-X₁₉-Y-X₂₀-A-X₂₁;
wherein X₉ is V, G, P, L, I, M, S, T, or A;
X₁₀ is R, S, T, Q, D, E, H, K, N, or A;
X₁₁ is H, R, K, G, T, S, N, Q, or A;
X₁₂ is G, P, V, L, I, M, S, T, or A;
X₁₃ is F, Y, W, V, L, I, G, or A;
X₁₄ is G, P, V, L, I, M, S, T, or A;
X₁₅ is N, Q, S, T, D, E, H, K, R, or A;
X₁₆ is S, G, T, M, N, Q, H, or A;
X₁₇ is Y, F, W, V, L, I, G, or A;
X₁₈ is I, G, P, V, L, M, S, T, or A;
X₁₉ is S, G, T, M, N, Q, H, or A;
X₂₀ is W, F, Y, V, L, I, G, or A; and
X₂₁ is Y, F, W, V, L, I, G, or A; and
wherein when CDR3-L comprises the amino acid sequence of SEQ ID NO: 3 then CDR3-H does not comprise the amino acid sequence of SEQ ID NO: 17.

2. The recombinant antibody or antigen binding fragment thereof of claim 1, wherein:
X₁ is V, G, L, I, or A;
X₂ is L, V, I, or A;
X₃ is Y, W, F, or A;
X₄ is S, G, T, or A;
X₅ is N, Q, D, E, or A;
X₆ is R, K, or A;
X₇ is V, G, L, I, or A;
X₈ is F, Y, W, or A;
X₉ is V, G, L, I, or A;
X₁₀ is R, K, or A;
X₁₂ is G, S, T, or A;
X₁₃ is F, Y, W, or A;
X₁₄ is G, S, T, or A;
X₁₅ is N, Q, D, E, or A;
X₁₆ is S, G, T, or A;
X₁₇ is Y, W, F, or A;
X₁₈ is I, V, L, or A;
X₁₉ is S, G, T, or A;
X₂₀ is W, Y, F, or A; and
X₂₁ is Y, W, F, or A.

3. The recombinant antibody or antigen binding fragment thereof of claim 1, wherein:
X₈ is F.

4. The recombinant antibody or antigen binding fragment thereof of claim 1, wherein:
X₁₀ is R;
X₁₁ is H;
X₁₃ is F;
X₁₈ is I;
X₁₉ is S; and
X₂₀ is W.

5. The recombinant antibody or antigen binding fragment thereof of claim 1, wherein CDR3-L comprises an amino acid sequence selected from SEQ ID NOs: 3, 5-6, 8-11, and 13-14.

6. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17; and
CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17.

7. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17; and
CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17.

8. The recombinant antibody or antigen binding fragment thereof of claim 1, wherein CDR3-H comprises an amino acid sequence selected from SEQ ID NOs: 17, 19-22, 24-30, and 32-33.

9. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32; and
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

10. The recombinant antibody or antigen binding fragment thereof of claim 1, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28; and
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33.

11. A recombinant antibody or antigen binding fragment thereof that comprises a CD3 binding domain, wherein the CD3 binding domain comprises an immunoglobulin light chain comprising complementarity determining regions (CDRs) CDR1-L, CDR2-L, and CDR3-L, and an immunoglobulin heavy chain comprising CDRs: CDR1-H, CDR2-H, and CDR3-H, wherein the immunoglobulin light chain and the immunoglobulin heavy chain comprise a set of CDR sequences selected from the group consisting of:
CDR1-L: SEQ ID NO: 1, CDR2-L: SEQ ID NO: 2 (GTK), CDR3-L: SEQ ID NO: 5, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 6, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 7, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 8, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 9, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 10, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 11, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 12, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 13, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 14, and CDR1-H: SEQ ID NO: 15, CDR2-H: SEQ ID NO: 16, CDR3-H: SEQ ID NO: 17;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 19;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 20;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 21;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 22;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 23;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 24;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 25;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 26;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 27;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 28;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 29;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 30;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 31;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 32;
CDR1-L: SEQ ID NO: 1, CDR2-L: SEQ ID NO: 2 (GTK), CDR3-L: SEQ ID NO: 3, and CDR1-H: SEQ ID NO: 15, CDR2-H: SEQ ID NO: 16, CDR3-H: SEQ ID NO: 33; and
CDR1-L: SEQ ID NO: 4, CDR2-L: SEQ ID NO: 2 (GTK), CDR3-L: SEQ ID NO: 5, and CDR1-H: SEQ ID NO: 18, CDR2-H: SEQ ID NO: 16, CDR3-H: SEQ ID NO: 178.

12. The recombinant antibody or antigen binding fragment thereof of claim 11, wherein the CD3 binding domain comprises a Fab, Fab′, (Fab′)₂, or a single chain variable fragment (scFv).

13. The recombinant antibody or antigen binding fragment thereof of claim 12, wherein the CD3 binding domain is the scFv.

14. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 37.

15. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 38.

16. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 39.

17. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 40.

18. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 41.

19. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 42.

20. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 43.

21. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 44.

22. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 45.

23. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 46.

24. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 47.

25. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 48.

26. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 49.

27. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 50.

28. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 51.

29. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 52.

30. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 53.

31. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 54.

32. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 55.

33. The recombinant antibody or antigen binding fragment of thereof of claim 11, wherein the CD3 binding domain comprises an amino acid sequence with at least 90%, 95%, 96%, 97%, 98%, 99%, or 100% identity to SEQ ID NO: 56.

34. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 57.

35. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 58.

36. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 59.

37. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 60.

38. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 61.

39. The recombinant antibody or antigen binding fragment thereof of claim 11, wherein the CD3 binding domain comprises an amino acid sequence with at least 90%, 95%, 96%, 97%, 98%, 99%, or 100% identity to SEQ ID NO: 63.

40. The recombinant antibody or antigen binding fragment thereof of claim 11, wherein the recombinant antibody or antigen binding fragment thereof further comprises an antigen binding domain.

41. The recombinant antibody or antigen binding fragment thereof of claim 40, wherein the antigen binding domain is a TROP2 binding domain.

42. The recombinant antibody or antigen binding fragment thereof of claim 41, wherein the TROP2 binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein:
CDR1-L comprises the amino acid sequence of SEQ ID NO: 64;
CDR2-L comprises the amino acid sequence of SEQ ID NO: 65 (SAS);
CDR3-L comprises the amino acid sequence of SEQ ID NO: 66;
CDR1-H comprises the amino acid sequence of SEQ ID NO: 67;
CDR2-H comprises the amino acid sequence of SEQ ID NO: 68, and
CDR3-H comprises an amino acid sequence selected from any one of SEQ ID NOs: 69-71.

43. The recombinant antibody or antigen binding fragment thereof of claim 42, wherein the TROP2 binding domain is a Fab.

44. The recombinant antibody or antigen binding fragment thereof of claim 42, wherein the CD3 binding domain is a scFv and the TROP2 binding domain is a Fab or Fab′.

45. The recombinant antibody or antigen binding fragment thereof of claim 42, wherein the immunoglobulin light chain of the TROP2 binding domain comprises an amino acid sequence with at least 90%, 95%, 96%, 97%, 98%, 99%, or 100% identity to SEQ ID NO: 72.

46. The recombinant antibody or antigen binding fragment thereof of claim 42, wherein the immunoglobulin heavy chain of the TROP2 binding domain comprises an amino acid sequence with at least 90%, 95%, 96%, 97%, 98%, 99%, or 100% identity to any one of SEQ ID NOs: 73-75.

47. The recombinant antibody or antigen binding fragment thereof of claim 42, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 73.

48. The recombinant antibody or antigen binding fragment thereof of claim 42, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 74.

49. The recombinant antibody or antigen binding fragment thereof of claim 42, wherein the immunoglobulin light chain of the TROP2 binding domain comprises an amino acid sequence with at least 90%, 95%, 96%, 97%, 98%, 99%, or 100% identity to SEQ ID NO: 72, and the immunoglobulin heavy chain of the TROP2 binding domain comprises an amino acid sequence with at least 90%, 95%, 96%, 97%, 98%, 99%, or 100% identity to SEQ ID NO: 75.

50. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 77.

51. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 78.

52. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 79.

53. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 80.

54. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 81.

55. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 82.

56. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 83.

57. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 84.

58. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 85.

59. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 86.

60. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 87.

61. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 88.

62. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 89.

63. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 90.

64. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 91.

65. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 92.

66. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 93.

67. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 94.

68. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 95.

69. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 96.

70. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 97.

71. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 98.

72. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 99.

73. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 100.

74. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 101.

75. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 105.

76. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 106.

77. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 107.

78. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 111.

79. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 112.

80. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 113.

81. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 109.

82. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 176.

83. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 72 and SEQ ID NO: 177.

84. The recombinant antibody or antigen binding fragment thereof of claim 11, wherein the recombinant antibody or antigen binding fragment thereof comprises the amino acid sequences of SEQ ID NO: 72 and SEQ ID NO: 105.

85. The recombinant antibody or antigen binding fragment thereof of claim 11, wherein the recombinant antibody or antigen binding fragment thereof comprises the amino acid sequences of SEQ ID NO: 72 and SEQ ID NO: 109.

86. The recombinant antibody or antigen binding fragment thereof of claim 11, wherein the recombinant antibody or antigen binding fragment thereof comprises the amino acid sequences of SEQ ID NO: 72 and SEQ ID NO: 176.

87. The recombinant antibody or antigen binding fragment thereof of claim 11, wherein the recombinant antibody or antigen binding fragment thereof comprises the amino acid sequences of SEQ ID NO: 72 and SEQ ID NO: 177.

88. The recombinant antibody or antigen binding fragment thereof of claim 40, wherein the antigen binding domain is an EGFR binding domain.

89. The recombinant antibody or antigen binding fragment thereof of claim 88, wherein the EGFR binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein:
CDR1-L comprises the amino acid sequence of SEQ ID NO: 115;
CDR2-L comprises the amino acid sequence of SEQ ID NO: 116 (YAS);
CDR3-L comprises the amino acid sequence of SEQ ID NO: 117;
CDR1-H comprises the amino acid sequence of SEQ ID NO: 118;
CDR2-H comprises the amino acid sequence of SEQ ID NO: 119; and
CDR3-H comprises the amino acid sequence of SEQ ID NO: 120.

90. The recombinant antibody or antigen binding fragment thereof of claim 89, wherein the immunoglobulin light chain of the EGFR binding domain comprises an amino acid sequence with at least 90%, 95%, 96%, 97%, 98%, 99%, or 100% identity to SEQ ID NO: 121, and the immunoglobulin heavy chain of the EGFR binding domain comprises an amino acid sequence with at least 90%, 95%, 96%, 97%, 98%, 99%, or 100% identity to SEQ ID NO: 122.

91. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 125 and SEQ ID NO: 126.

92. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 127 and SEQ ID NO: 128.

93. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 129 and SEQ ID NO: 130.

94. The recombinant antibody or antigen binding fragment thereof of claim 11, wherein the recombinant antibody or antigen binding fragment thereof comprises the amino acid sequences of SEQ ID NO: 125 and SEQ ID NO: 126.

95. The recombinant antibody or antigen binding fragment thereof of claim 11, wherein the recombinant antibody or antigen binding fragment thereof comprises the amino acid sequences of SEQ ID NO: 127 and SEQ ID NO: 128.

96. The recombinant antibody or antigen binding fragment thereof of claim 40, wherein the antigen binding domain is a PSMA binding domain.

97. The recombinant antibody or antigen binding fragment thereof of claim 96, wherein the PSMA binding domain comprises an immunoglobulin light chain comprising CDRs: CDR1-L, CDR2-L, and CDR3-L, and an immunoglobulin heavy chain comprising CDRs: CDR1-H, CDR2-H, and CDR3-H, wherein:
CDR1-L comprises the amino acid sequence of SEQ ID NO: 131;
CDR2-L comprises the amino acid sequence of SEQ ID NO: 132 (EAS);
CDR3-L comprises the amino acid sequence of SEQ ID NO: 133;
CDR1-H comprises the amino acid sequence of SEQ ID NO: 134;
CDR2-H comprises the amino acid sequence of SEQ ID NO: 135; and
CDR3-H comprises the amino acid sequence of SEQ ID NO: 136.

98. The recombinant antibody or antigen binding fragment thereof of claim 97, wherein the immunoglobulin light chain of the PSMA binding domain comprises an amino acid sequence with at least 90%, 95%, 96%, 97%, 98%, 99%, or 100% identity to SEQ ID NO: 137, and the immunoglobulin heavy chain of the PSMA binding domain comprises an amino acid sequence with at least 90%, 95%, 96%, 97%, 98%, 99%, or 100% identity to SEQ ID NO: 138.

99. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 141 and SEQ ID NO: 142.

100. The recombinant antibody or antigen binding fragment thereof of claim 11, wherein the recombinant antibody or antigen binding fragment thereof comprises amino acid sequences with at least 90%, 95%, 96%, 97%, 98%, 99%, or 100% identity to SEQ ID NO: 143 and SEQ ID NO: 144.

101. The recombinant antibody or antigen binding fragment thereof of claim 11, the recombinant antibody or antigen binding fragment thereof comprises amino acid sequences with at least 90%, 95%, 96%, 97%, 98%, 99%, or 100% identity to SEQ ID NO: 145 and SEQ ID NO: 146.

102. The recombinant antibody or antigen binding fragment thereof of claim 11, wherein the recombinant antibody or antigen binding fragment thereof comprises the amino acid sequences of SEQ ID NO: 141 and SEQ ID NO: 142.

103. The recombinant antibody or antigen binding fragment thereof of claim 11, wherein the recombinant antibody or antigen binding fragment thereof comprises the amino acid sequences of SEQ ID NO: 143 and SEQ ID NO: 144.

104. The recombinant antibody or antigen binding fragment thereof of claim 40, wherein the CD3 binding domain and the antigen binding domain are connected by a linker.

105. The recombinant antibody or antigen binding fragment thereof of claim 104, wherein the linker comprises an amino acid sequence selected from any one of SEQ ID NOs: 147-175.

106. A pharmaceutical composition comprising:
(i) the recombinant antibody or antigen binding fragment thereof of claim 11; and
(ii) a pharmaceutically acceptable excipient.

107. An isolated recombinant nucleic acid molecule encoding a polypeptide of the recombinant antibody or antigen binding fragment thereof of claim 11.

108. A vector comprising the isolated recombinant nucleic acid molecule of claim 107.

109. A host cell comprising the isolated recombinant nucleic acid molecule of claim 107.

110. A method of treating cancer in a subject in need thereof comprising administering to the subject the recombinant antibody or antigen binding fragment thereof of claim 11.

111. A kit comprising the pharmaceutical composition of claim 106.
